(19) United States
(12) Patent Application Publication
(10) Pub. No.: US 2024/0141294 A1
(43) Pub. Date: May 2, 2024

(54) CGMP COMPLIANT PRODUCTION AND EXPANSION OF PLASMACYTOID DENDRITIC CELLS FROM HEMATOPOIETIC STEM AND PROGENITOR CELLS

(71) Applicant: AARHUS UNIVERSITET, Aarhus C (DK)

(72) Inventors: Anders LAUSTSEN, Aarhus V (DK); Martin Roelsgaard JAKOBSEN, Risskov (DK); Rasmus Otkjær BAK, Brabrand (DK)

(21) Appl. No.: 18/574,238

(22) PCT Filed: Jun. 30, 2022
(86) PCT No.: PCT/EP2022/068023

§ 371 Date: Dec. 26, 2023

(30) Foreign Application Priority Data
Jul. 2, 2021 (EP) 21183430.4

(51) Int. Cl. C12N 5/0784

(52) U.S. Cl. CPC C12N 5/0639; C12N 2500/38; C12N 2500/90; C12N 2501/24; C12N 2506/11

(57) ABSTRACT

HSPC-derived Plasmacytoid dendritic cells (HSPC-pDCs) constitute a rare type of immune cell with multifaceted functions that bridge pivotal pants of the immune system. Biological studies of blood-derived HSPC-pDCs and their potential use as a cell-based immunotherapy have long been challenged by the scarce amounts of HSPC-pDCs that can be extracted from blood samples. This invention is related to a process for HSPC-pDC production applicable for clinical use, which involves in vitro differentiation of hematopoietic stem and progenitor cells (HSPCs). With this optimized GMP-compliant protocol, we generated an average of 465 million HSPC-derived pDCs (HSPC-pDCs) starting from 100,000 cord-blood derived HSPCs, and we also show that the protocol enables robust HSPC-pDC generation from HSPCs extracted from whole blood. The produced cells display a pDC phenotype (Lin−/CD11c−/CD123+/CD303+) and the ability to produce high levels of type I interferon upon TLR7 and TLR9 stimulation.

### TECHNICAL FIELD OF THE INVENTION

The present invention relates to a process for the production and expansion of HSPC-derived plasmacytoid dendritic cells (HSPC-pDCs) from hematopoietic stem and progenitor cells (HSPCs). In particular, the present invention relates to such a process carried out under CGMP compliant conditions and cells obtained from such process.

### BACKGROUND OF THE INVENTION

Plasmacytoid dendritic cells (pDCs) represent a rare and unique type of immune cell that plays a central role particularly in the detection and control of viral infections. In addition to conventional dendritic cell (cDCs) functions, pDCs are capable of producing high levels of type I interferon (IFN) upon exposure to virus-derived nucleic acids that are recognized by Toll-like Receptor (TLR) 7 and TLR9 [1]. Though the signature cytokine secreted by activated pDCs is type I IFNs, pDCs also effectively produce other pro-inflammatory cytokines and chemokines such as IL-1β, IL-6, IL-8, TNFα, and ligands for CXCR3 (CXCL9, CXCL10, and CXCL11) [2]. Consequently, pDCs have emerged as key effectors and regulators within the immune system, and their implication within a number of diseases, as well as their potential clinical application, have become topics of great interest. Several preclinical studies have confirmed the immunotherapeutic potential of pDCs for the treatment of cancer through a multi-faceted stimulation of the immune system [3, 4]. Importantly, two clinical trials have shown that autologous tumor antigen-loaded pDCs induce anti-tumoral responses and significantly improved clinical outcome for melanoma and prostate cancer patients, respectively [5, 6]. In one of these trials, a mixture of pDCs and cDCs were used, and a follow-up comparison of these two cell types suggests that pDCs are superior to cDCs at attracting CD8⁺ T cells, γ/δ T cells, and CD56⁺ NK cells to sites of melanoma [2]. Overall, this indicates that pDC-based anti-cancer immunotherapy could be an alternative or supplement to current cancer immunotherapy. While attempts have been made to translate the use of pDCs into a clinical immunotherapeutic setting, their use have been severely impeded by their rarity within peripheral blood (0.1%+/−0.07% of PBMCs) [7]. Coupled with their short ex vivo survival, they are very difficult to study and modulate as a population [8]. To this end, a few efforts have been made to generate pDCs ex vivo by differentiation of CD34⁺ hematopoietic stem and progenitor cells (HSPCs) [9]. These studies demonstrate that HSPC-derived pDCs (HSPC-pDCs) can be generated from different sources of HSPCs, including cord blood (CB) and mobilized peripheral blood. Although some improvements in different methods of pDC generation have been achieved, adoptive transfer therapy of autologous HSPC-pDCs is still challenging due to low cell yield and the requirement for patients to undergo G-CSF-stimulated HSPC mobilization.

Recently, we reported a novel robust ex vivo setup for generating high numbers of pDCs from HSPCs [10]. We identified that a combination of growth factors, cytokines, and small molecules (Flt3-L, TPO, SCF, IL-3, and SR1) supported HSPC expansion and differentiation into immature HSPC-pDCs. Following a 21-day culture period, an average of 35% of the culture was HSPC-pDCs, which could be enriched to near-purity using immunomagnetic depletion of non-pDCs. Most importantly, we showed that to generate a mature and functional phenotype the HSPC-pDCs culture required priming by exposure to type I and II IFNs [10].

For the setup described above to be clinical relevant, it has to be carried out under current good manufacturing process (CGMP) standards. Thordardottir et al applied part of the setup in a CGMP setting where hematopoietic stem cells were differentiated into plasmacytoid dendritic cells [9] but was not able to setup a complete CGMP protocol.

Hence, a setup where the entire process is carried out in a CGMP setting would be advantageous. More advantageous would be a CGMP compliant setting producing high amounts of active and mature pDCs.

### SUMMARY OF THE INVENTION

In here is presented a clinical-relevant strategy to increase HSPC expansion and promote the functionality and numbers of generated HSPC-pDCs under current good manufacturing process (CGMP) standards. HSPC pre-expansion with the pyrimido-indole derivative UM171 combined with low-density culturing highly promoted expansion of HSPC-pDCs. We demonstrate that commercially available CGMP medium fails to produce HSPC-pDCs with functional capacity to produce key cytokines such as type I IFN upon TLR7 and TLR9 activation—a hallmark for pDC functionality. Importantly, we discovered that supplementing culture conditions with the usage of ascorbic acid rescued the functionality of pDCs, thereby establishing ascorbic acid as an essential culture and differentiation component for the generation of HSPC-pDCs. Finally, we show that such combined efforts enable the generation of HSPC-pDCs from naturally circulating HSPCs obtained from peripheral blood (cHSPC). Collectively, we present a novel platform that allows CGMP-compliant generation of therapeutically relevant numbers of HSPC-pDCs from HSPCs obtained from standard blood samples without the need for mobilization regiments like G-CSF and plerixafor.

Example 2 shows that low density expansion of HSPCs increases yield of pDCs.

Example 5 shows that the ability of the pDCs to produce type I IFN upon stimulation with TLR7 or TLR9 agonists are drastically reduced when grown in commercially available CGMP media, compared to non-CGMP media.

Example 6 shows how supplementing CGMP media with ascorbic acid improves the expansion, differentiation, and activation of the cells to a level comparable to non-CGMP media.

Example 8 shows that the process of the invention provides HSPC-pDCs with an overall unique expression profile.

Example 9 shows that the HSPC-pDCs according to the invention also have unique expression profile for TLR7 and TLR9 pathway-related genes. Such a changed expression profile is considered particularly relevant for the HSPC-pDCs.

Example 10 shows the effect of SR1 and IL-3 on HSPC-pDC cell growth, phenotype, and functionality.

Examples 11 and 12 surprisingly show that differentiated pDCs can be cryopreserved after differentiation for long-term storage, thawed, and primed without greatly impacting their phenotype and functionality compared with fresh HSPC-pDCs. Further, these examples show that HSPC-pDCs can be primed prior to cryopreservation and thawed while maintaining their phenotype and the ability to respond to TLR stimulation.

An advantage of the discovery that cryopreservation after priming is possible, is that the production of efficient “ready-to-use” (of-the-shelf) product is possible. Thus, HSPC-pDCs can be produced at a dedicated cell production facility and subsequently shipped (frozen) to the site of use, such as a hospital.

Thus, an object of the present invention relates to provision of mature and functional HSPC-pDCs by ex vivo differentiation of hematopoietic stem and progenitor cells (HSPCs) that solves the above-mentioned problems.

In particular, it is an object of the present invention to provide mature and functional HSPC-pDCs differentiated from hematopoietic stem and progenitor cells (HSPCs) under Good manufacture practice (GMP) for the pDCs to be used in a clinical setting.

Yet an object is the provision of “ready-to-use” (of-the-shelf) HSPC-pDC products.

In an aspect, the invention relates to a process for producing HSPC-derived Plasmacytoid dendritic cells (HSPC-pDCs) from hematopoietic stem and progenitor cells (HSPCs), the process comprising the steps:
a) providing hematopoietic stem and progenitor cells (HSPCs);
b) differentiating said HSPCs, to generate precursor-HSPC-pDCs; and
c) priming said precursor-HSPC-pDCs with interferon to provide mature HSPC-pDCs;
   wherein step b) and step c) are carried out in serum-free medium comprising ascorbic acid, preferably the serum-free medium is a CGMP-compliant medium.

In a preferred embodiment, step c) includes the steps of
freezing the generated precursor-HSPC-pDCs after priming; or
freezing, storing and thawing the generated precursor-HSPC-pDCs before priming; or
freezing, storing and thawing the generated precursor-HSPC-pDCs after priming.

As shown in examples 11 and 12, freezing (cryopreservation) is possible before or after priming, while preserving function.

Another aspect of the invention relates to a process for producing HSPC-derived Plasmacytoid dendritic cells (HSPC-pDCs) from hematopoietic stem and progenitor cells (HSPCs), the process comprising the steps:
a) providing hematopoietic stem and progenitor cells (HSPCs);
b) differentiating said HSPCs, to generate precursor-HSPC-pDCs;
c) priming said precursor-HSPC-pDCs with interferon to provide mature pDCs; and
d) activating the mature pDCs to induce secretion of type I interferon;
   wherein steps b)-d) are carried out in serum-free medium comprising ascorbic acid, preferably the serum-free medium is a CGMP-compliant medium.

A further aspect of the present invention relates to HSPC-pDCs obtained/obtainable by a process according to the present invention.

Yet a further aspect relates to isolated HSPC-pDC cells, which
express one or more genes selected from the group consisting of AP3S2, CLEC4C, FCER1G, IRF7, IRF8, LAMP5, LILRA4, MYD88, NRP1, PACSIN1, PLSCR1, TLR7, TLR8, TLR9, TRAF3, UBE2N, and UNC93B1; and/or
express an increased level of one or more genes selected from the group consisting of AP3S2, CLEC4C, FCER1G, IRF7, IRF8, LAMP5, LILRA4, MYD88, NRP1, PACSIN1, PLSCR1, TLR7, TLR8, TLR9, TRAF3, UBE2N, and UNC93B1 compared to HSPC-pDC isolated from blood; and/or
express an increased level of one or more genes selected from the group consisting of AP3S2, CLEC4C, FCER1G, IRF7, IRF8, LAMP5, LILRA4, MYD88, NRP1, PACSIN1, PLSCR1, TLR7, TLR8, TLR9, TRAF3, UBE2N, and UNC93B1 compared to HSPC-pDCs produced from hematopoietic stem and progenitor cells (HSPCs), in the absence of ascorbic acid; and/or
express an increased level of the one or more genes selected from the group consisting of AP3S2, CLEC4C, FCER1G, IRF7, IRF8, LAMP5, LILRA4, MYD88, NRP1, PACSIN1, PLSCR1, TLR7, TLR8, TLR9, TRAF3, UBE2N, and UNC93B1 compared to HSPC-pDCs produced in the absence of ascorbic acid in step b) or c) or d), preferably in absence of ascorbic acid in step b)-d).

Yet another aspect of the present invention relates to the use of ascorbic acid in CGMP serum-free medium for providing viability and expansion of HSPCs, and for promoting the development and functionality of HSPC-pDCs, such as the secretion of type I IFN following activation.

Thus, in an aspect the step of activating the mature HSPC-pDCs are non-essential, since non-activated HSPC-pDCs may also have commercial use. In a preferred embodiment of this aspect, step b) comprises low density expansion of HSPCs to increase yield of HSPC-pDCs. Example 2 shows that low density expansion of HSPCs increases yield of HSPC-pDCs.

In another preferred embodiment of this aspect, steps b)-c) are carried out in serum-free medium comprising ascorbic acid, preferably a (serum-free) CGMP-compliant medium.

In a preferred embodiment, the invention provides a process for producing HSPC-derived plasmacytoid dendritic cells (HSPC-pDCs) from hematopoietic stem and progenitor cells (HSPCs), the process comprising the steps:
a) providing a human peripheral blood sample comprising hematopoietic stem and progenitor cells (HSPCs), preferably which has been obtained from a subject that has not been administered a mobilisation agent such as G-CSF or plerixafor;
b1) pre-expanding the hematopoietic stem and progenitor cells (HSPCs) provided in step a) starting with a concentration of 0.1-0.5×10⁶ cells/mL for up to 8 days; preferably in the presence of UM171 and/or StemRegenin 1;
b2) differentiating the pre-expanded cells from step b1) to generate precursor-pDCs; and
c) priming said precursor-HSPC-pDCs with interferon to provide mature HSPC-pDCs, preferably wherein the priming medium comprises P/S or IL-3;
d) optionally also activating the mature HSPC-pDCs to induce secretion of type I interferon, preferably in the presence of a TLR7 agonist and/or a TLR9 agonist,
   wherein step b1), step b2), step c) and optional step d) are carried out in serum-free medium comprising ascorbic acid, preferably a (serum-free) CGMP-compliant medium.

In further preferred embodiments, the invention provides HSPC-pDCs obtained by the above method and their use in treating disease, particular cancer or autoimmune disease.

In a further preferred embodiment, the invention provides a method of preparing a therapeutic composition comprising:
a) providing a human peripheral blood sample comprising hematopoietic stem and progenitor cells (HSPCs), preferably which has been obtained from a subject that has not been administered a mobilisation agent such as G-CSF or plerixafor;
b1) pre-expanding the hematopoietic stem and progenitor cells (HSPCs) provided in step a) starting with a concentration of 0.1-0.5×10⁶ cells/mL for up to 8 days; preferably in the presence of UM171 and/or StemRegenin 1; b2) differentiating the pre-expanded cells from step b1) to generate precursor-pDCs; and
c) priming said precursor-HSPC-pDCs with interferon to provide mature HSPC-pDCs, preferably wherein the priming medium comprises P/S or IL-3;
d) optionally also activating the mature HSPC-pDCs to induce secretion of type I interferon, preferably in the presence of a TLR7 agonist and/or a TLR9 agonist,
e) optionally loading the mature HSPC-derived-pDCs with antigens, such as tumour antigens, or transforming the mature HSPC-derived pDCs with an exogenous construct, such as a CAR-T construct; and
f) formulating said mature HSPC-derived-pDCs into a therapeutic composition, wherein step b1), step b2), step c) and optional step d) are carried out in serum-free medium comprising ascorbic acid, preferably a (serum-free) CGMP-compliant medium.

### BRIEF DESCRIPTION OF THE FIGURES

FIG. 1 shows that Lower HSPC density increases expansion of HSPCs during HSPC-pDC differentiation. HSPCs were thawed and 2×10⁵ cells were cultured for 21 days using the standard cultivation protocol described previously (SCP), or with a low-density protocol (LD). a) Table showing the density cells were split to during pDC differentiation. b) Density of cells during pDC differentiation prior to medium change. c) Viability of isolated HSPC-pDCs. d) Viability of cells during pDC differentiation. e) The total cumulative number of cells during culture were measured. To maintain culture format and minimize costs, a fraction of the culture was continuously discarded during passaging, which was taken into account when calculating cumulative cell numbers. f) The number of HSPC-pDCs isolated after immunomagnetic negative selection was determined at day 21 and total cumulative number of HSPC-pDCs generated was calculated based on the fraction of cells discarded during culture. g) Percentage of HSPC-pDCs of the total cell population at the day of isolation. h) Isolated HSPC-pDCs were primed with type I IFN for three days or left unprimed, after which they were stimulated for 20 hours with agonists directed against TLR7 (R837) or TLR9 (CpG-A) and type I IFN was measured. Data shown represent mean of two cord blood donors.

FIG. 2 shows that Serum-free conditions improve expansion of HSPCs and HSPC-pDCs isolated at earlier time points retain a functional phenotype. HSPCs were thawed and 1×10⁵ cells were cultured in RPMI or SFEM II at a density of 0.5-5×10⁶ cells/mL. HSPC-pDC isolation was performed after 16, 18 and 21 days of culture and cryo-preserved. HSPC-pDCs were later thawed, primed for three days and subsequently phenotypically analyzed. a) Table showing days were cells were split to a new density (between 0.5-5×10⁶ cells). b) Cell density of HSPCs during HSPC-pDC differentiation prior to medium change. c) Calculated numbers of HSPCs during culture. Arrows indicate days when HSPC-pDCs were isolated. d) Numbers of isolated HSPC-pDCs. e) Proportion HSPC-pDCs within the total population of cells at the day of isolation. f) Viability of isolated HSPC-pDCs. g-h) Type I IFN response of non-primed or primed HSPC-pDCs after activation with the TLR7 agonist R837 (g) or the TLR9 agonist CpG 2216 (h). i-j) Surface expression of CD123 (i) and CD303 (j) on non-primed or primed HSPC-pDCs (gated on lineage negative, CD11c negative cells). Data shown represent±SEM of three donors and three donors each analyzed in technical triplicates.

FIG. 3 shows that Pre-expansion of HSPCs increases the yield of HSPC-pDCs. a) Schematic representation showing generation of HSPC-pDCs from pre-expanded HSPCs. HSPCs were pre-expanded at low density (1-5×10⁵ cells/mL) in SFEM II medium supplemented with UM171 for 4, 6 or 8 days and then cryo-preserved. Cells were then thawed, phenotyped for CD34 and 1×10⁵ HSPCs were seeded for HSPC-pDC generation. HSPC-pDCs were isolated after either 16 or 21 days of culture and phenotypically analyzed. b) HSPCs density during pre-expansion. Arrows indicate points were HSPCs were cryo-preserved c) Calculated number of HSPCs during pre-expansion. Arrows indicate points were HSPCs were cryo-preserved. d) Calculated number of cells during HSPC-pDC differentiation using pre-expanded HSPCs without the pre-expansion factor taken into account (same starting cell number at differentiation). e) Percentage of HSPC-pDCs of total population of cells. f) Calculated number of HSPC-pDCs isolated after 16 and 21 days of culture with fold pre-expansion taken into account. g-h) Levels of type I IFN from HSPC-pDCs after stimulation with the TLR7 agonist R837 (g) or the TLR9 agonist CpG-2216 (h). Data shown represent±SEM of four donors and four donors each analyzed in technical triplicates.

FIG. 4 shows that Ascorbic is required for generation of functional HSPC-pDCs with DC medium. a-e) 1×10⁵ HSPCs were cultured in SFEM II, the CGMP-compliant DC medium (GMP (DC)) or the CGMP-compliant SCGM (GMP (SCGM)). For all conditions, cells were kept at a density of 0.5-5×10⁶ cells/mL throughout culture. HSPC-pDCs were isolated after 21 days of culture and phenotypically and functionally analyzed. a) Calculated number of cells during HSPC-pDC differentiation. b) Viability of cells during HSPC-pDC differentiation. c) Calculated number of isolated HSPC-pDCs after 21 days of culture. d) Percentage of HSPC-pDCs of total population of cells. e) Type I IFN response of HSPC-pDCs after stimulation with the TLR7 agonist R837 or the TLR9 agonist CpG-2216.

FIG. 5 shows that ascorbic acid medium supplementation is required for HSPC-pDC generation using the CGMP compliant DC medium. HSPCs were thawed and 1×10⁵ cells were seeded in SFEM II, the CGMP-compliant medium DC medium (GMP (DC)) or DC medium supplemented with ascorbic acid (GMP (DC)+AA). For all conditions, cells were kept at a density of 0.5-5×10⁶ cells/mL throughout culture. HSPC-pDCs were isolated after 16 and 21 days of culture and phenotypically analyzed. a) Calculated number of total cells during HSPC-pDC differentiation. b) Viability of HSPC-pDCs isolated after 16 or 21 days of culture. c) Calculated number of HSPC-pDCs after isolation at 16 days or 21 days of culture. d) Percentage of HSPC-pDCs of total cells. d) Percentage of HSPC-pDCs of total cells. e-f) Type I IFN response of HSPC-pDCs isolated after 16 or 21 days of culture after activation with the TLR7 agonist R837 (e) or the TLR9 agonist CpG-2216 (f). g-h) Surface expression of CD123 (g) and CD303 (h) on non-primed or primed HSPC-pDCs (gated on lineage negative, CD11c negative cells). Data shown represent±SEM of four donors and ±SEM of four donors each analyzed in technical triplicates.

FIG. 6 shows generation of HSPC-pDCs from HSPCs from peripheral whole blood using optimized CGMP-compliant medium. HSPCs were pre-expanded for 4 days at low density (1-5×10⁵ cells/mL) in CGMP-compliant medium (SCGM) supplemented with UM171 and then cryo-preserved. Subsequently, cells were thawed, phenotyped for CD34, and 1×10⁵ HSPCs were seeded for HSPC-pDC generation. HSPC-pDCs were isolated after 16 days of culture and phenotypically analyzed. a) Calculated number of cells during HSPC-pDC differentiation using pre-expanded HSPCs (without the pre-expansion factor taken into account). b) Calculated number of HSPC-pDCs upon isolation of HSPC-pDCs at 16 days of culture (with fold pre-expansion taken into account). c) Percentage of HSPC-pDCs of the total population of cells. d-e) Levels of type I IFN upon stimulation of HSPC-pDCs with the TLR7 agonist R837 (d) or the TLR9 agonist CpG-2216 (e). f) Type I IFN response of HSPC-pDCs generated from cHSPCs using either SFEM II medium, DC medium or DC medium supplemented with AA. HSPC-pDCs were activated with either the TLR7 agonist R837 (f) or the TLR9 agonist CpG-2216 (g). Data shown represent±SEM of four donors (a-c), four donors each analyzed in technical triplicates (d-e) and one donor analyzed in technical triplicates (f-g).

FIG. 7 shows a schematic illustration showing the collective procedure of generating cHSPC-pDC for therapeutic purposes starting from a patient blood sample. CD34⁺ cHSPCs are initially isolated using immunomagnetic selection. cHSPCs are then pre-expanded at low density using small molecule inhibitors that promote self-renewal. Subsequently, pre-expanded cHSPCs are differentiated into cHSPC-pDCs that can either be readily used for immunotherapeutic purposes or cryo-preserved to allow for multiple vaccine regiments.

FIG. 8 shows the RNA-seq profile of HSPC-pDCs generated with ascorbic acid. (a) Volcano plot showing differentially-expressed genes in the HSPC-pDCs generated with ascorbic acid compared to HSPC-pDCs generated without ascorbic acid. The threshold for up- and downregulation was set at |log 2FC|>=1 and Qvalue<=0.05 as indicated by the dashed lines. (b) Gene ontology bubble chart displaying the 20 most enriched biological processes for the differentially expressed genes in HSPC-pDCs generated with ascorbic acid. The x-axis shows the enrichment ratio (rich ratio), which is the ratio between the number of differentially expressed genes within the biological process and the number of total genes annotated in that process. The size of the bubble represents the number of differentially expressed genes within the process and the color represents the statistical significance of the enrichment.

FIG. 9 shows removal of SR1 and/or IL-3 during the final 3 days of HSPC-pDC differentiation influence cell growth, phenotype, and functionality. A) Absolute change in the number of cells in culture between day 14 and 17, where cells were deprived of IL-3 and/or SR1. B) Fold change in the number of cells in culture between day 14 and 17. C) Viability of cells during HSPC-pDC differentiation D-E) HSPC-pDCs were primed with type I IFN for 24 hours or left unprimed. Following the immunophenotype was assessed with flow cytometry. Surface expression of CD123 (D) and CD303 (E) on HSPC-pDCs (gated on lineage negative, CD11c negative cells). F) Primed HSPC-pDCs were stimulated for 20 hours with agonists directed against TLR7 (R837+R848) or TLR9 (CpG-A) and IFNα in the media was measured with ELISA. Data shown represent mean of two cord blood donors, each collected as collected as biological duplicates. Data shown represents the mean+SEM (error bars) of the two cord-blood donors.

FIG. 10 shows that HSPC-pDC maintain their phenotype and functionality after cryopreservation. Cord blood HSPCs were thawed and 1×10⁵ cells were seeded in CGMP-compliant medium DC medium supplemented with ascorbic acid. The cells were kept at a density of 0.5-3×10⁶ cells/mL throughout culture. Bulk HSPC-pDCs were harvested after 16 days of culture and phenotypically analyzed or cryopreserved for later phenotypical analysis. A) Viability and recovery of cryopreserved HSPC-pDCs after thawing. N=11 donors. B) Purity of fresh vs cryopreserved (cryo) IFN-primed or unprimed HSPC-pDCs. Purity was determined by flow cytometry after 24h of IFN-priming (gated on Live, Lineage^{neg} (CD3, CD14, CD16, CD19, CD20, CD56) and CD11c^{neg}). N=5.C) Frequency of unprimed or primed and fresh or cryopreserved (cryo) HSPC-pDCs expressing CD123, CD303, or CD304. The immunophenotype was determined by flow cytometry (gated on Live, Lineage^{Neg}, CD11c^{Neg} cells). Statistical analysis: Two-way ANOVA with Tukey's multiple comparisons test (N=5). D) Frequency HSPC-pDCs double positive for CD123/CD303, CD123/CD304, and CD303/CD304. Conditions as described in C). E-H) Type I IFN response of fresh or cryopreserved HSPC-pDCs after no treatment (E) or activation with the TLR7 agonist R837 (F), TLR7/8 agonist R848 (G), or the TLR9 agonist CpG-2216 (h). N=5. Data are depicted as mean+SD.

FIG. 11 compares priming of HSPC-pDC (before or after cryopreservation. A) Schematic overview of generation and cryopreservation of untreated or primed HSPC-pDCs (top panel). Cord blood HSPCs were thawed and 1×10⁵ cells were seeded in CGMP-compliant medium DC medium supplemented with ascorbic acid. The cells were kept at a density of 0.5-3×10⁶ cells/mL throughout culture. At day 15 a subset of the culture is primed (pre-primed) with IFNs in the differentiation medium. Bulk pre-primed or unprimed HSPC-pDCs were harvested after 16 days of culture and cryopreserved for later phenotypical analysis. The below panel shows a schematic overview of the phenotypical comparison HSPC-pDCs primed before (pre-primed) or after cryopreservation (standard). B) The fold expansion of the cells during HSPC-pDC differentiation. At day 15 the culture was split into a primed and unprimed fraction, thus the expansion on day 16 has been calculated based on the expansion of the individual conditions (N=2 donors). C) Viability and recovery of untreated (UT) or pre-primed cryopreserved HSPC-pDCs after thawing (3 donors). D) Frequency of immunophenotypically marker expression on primed (before cryopreservation) or primed (after cryopreservation) HSPC-pDCs determined by flow cytometry. Top panel shows the frequency of CD123^{(pos)}/CD304^{(pos)} HSPC-pDCs (gated on Live, Lineage^{Neg}, CD11c^{Neg} ells). Panel below shows the frequency of CD40, CD80, and CD85 positive HSPC-pDCs (gated on Live, Lineage^{Neg}, CD11c^{Neg}, CD304^{Pos} cells). N=2. E) Type I IFN response of primed or pre-primed HSPC-pDCs after activation with the TLR7 agonist R837 (Top left), TLR7/8 agonist R848 (Top right), or the TLR9 agonist CpG-2216 (below). N=3. Data are depicted as mean+SD.

The present invention will now be described in more detail in the following.

### DETAILED DESCRIPTION OF THE INVENTION

### Definitions

Prior to discussing the present invention in further details, the following terms and conventions will first be defined:

Hematopoietic Stem and Progenitor Cells (HSPCs)

Hematopoietic stem and progenitor cells consist of multipotent stem cells capable of giving rise to all types of blood cells, including lymphoid and myeloid lineages. They also contain progenitor cells capable of giving rise to different cells within a certain blood lineage. Lymphoid lineages include cell types such as NK cells, B

HSPC-Derived-Plasmacytoid Dendritic Cells (HSPC-pDCs)

HSPC-derived Plasmacytoid dendritic cells (HSPC-pDCs) are a type of pDCs derived from hematopoietic stem and progenitor cells (HSPCs). pDCs are an unique autonomous cell type that do not fall within the family of conventional dendritic cells (cDCs). pDCs are distinct from cDCs by a set of surface markers, such as the lack of CD11c, and the expression of CD123, CD303, CD304 and HLA-DR. pDCs primarily sense pathogens through TLR7 or TLR9, leading to the production of high levels of type I IFN, and pro-inflammatory factors. pDCs are also capable of processing, and presenting antigens and activating T cells, and inducing direct cell-mediated killing through TRAIL.

Mature HSPC-pDCs

Mature HSPC-pDCs are precursor HSPC-pDCs, which have undergone a priming step, were precursor HSPC-pDCs are seeded out in medium supplemented with e.g. type I and II IFNs, leading to a clear maturation step of the cells functionality.

CGMP

CGMP refers to the Current Good Manufacturing Practice regulations enforced by the FDA. CGMPs provide for systems that assure proper design, monitoring, and control of manufacturing processes and facilities. Adherence to the CGMP regulations assures the identity, strength, quality, and purity of drug products by requiring that manufacturers of medications adequately control manufacturing operations. This includes establishing strong quality management systems, obtaining appropriate quality raw materials, establishing robust operating procedures, detecting and investigating product quality deviations, and maintaining reliable testing laboratories. This formal system of controls at a pharmaceutical company, if adequately put into practice, helps to prevent instances of contamination, mix-ups, deviations, failures, and errors. This assures that drug products meet their quality standards.

Serum-Free

In the present context, the term “serum-free” refers to a composition or medium being free from blood serum, such as free from fetal bovine serum (FBS) and human serum.

CGMP-Compliant Medium

For cell mediums, CGMP is a mandatory step for clinical translation. Xenogenic serum (e.g. FBS) and human serum carries the risk of contamination with infectious agents such as viruses and prions. Furthermore, the composition and activity of individual serum batches are prone to high variation.

In certain embodiments, a medium for use in the invention is a priming medium or a serum-free medium. In certain such embodiments, the medium is sterile, free from contaminants, and consists of a defined set of components. Such media may be equivalent to CGMP-compliant media, CGMP media and CGMP serum-free media.

Priming

In the present context the term “priming” is to be understood as a specific part of the HSPC-pDC generation setup, were precursor HSPC-pDCs are ‘primed’ to become functionally mature HSPC-pDCs. Functionally active HSPC-pDCs express pDC markers, such as CD123, CD303, CD304 and HLA-DR, and responds to TLR7 and TLR9 agonists. Specifically, pre-cursor HSPC-pDCs are seeded in medium in the absence of specific growth factors, such as Flt3-L, SCF and TPO and SR1. The growth factors and molecules IL-3, P/S and ascorbic are kept in the medium, and the cells are primed with type I and II IFNs for a period of three days, resulting in their functional maturation.

Activating

In the present context the term “activating” is to be understood as the stimulation of HSPC-pDCs with specific agonists directed against receptors, such as TLR7, TLR9, RIG-I, or STING, leading to the activation of the HSPC-pDCs. Downstream signaling will induce ‘activation’ of the pDCs, which is reflected in for example the secretion of type I IFNs and pro-inflammatory factors, such as IL-6 and TNF-α, and the up-regulation of different surface receptors, such as CD40 and CD80. The activation can be performed on both non-primed and primed pDCs to assess if they are active. Activation can also be performed to increase the ability of the HSPC-pDCs to take up antigens, and present and induce the activation of T cells, and perform cell-mediated killing.

Cryopreservation

“Cryopreservation” is a process where the cells are preserved by cooling to very low temperatures (typically −80° C. using solid carbon dioxide or −196° C. using liquid nitrogen).

Process for Producing HSPC-Derived Plasmacytoid Dendritic Cells (HSPC-pDCs)

As outlined above, the present invention relates to production of plasmacytoid dendritic cells (pDCs) from hematopoietic stem and progenitor cells (HSPCs) according to good manufacture procedure.

Thus, an aspect the present invention relates to a process for producing HSPC-derived Plasmacytoid dendritic cells (HSPC-pDCs) from hematopoietic stem and progenitor cells (HSPCs), the process comprising the steps:
a) providing hematopoietic stem and progenitor cells (HSPCs);
b) differentiating said HSPCs, to generate precursor-HSPC-pDCs; and
c) priming said precursor-HSPC-pDCs with interferon to provide mature HSPC-pDCs,
   wherein steps b) and step c) are carried out in serum-free medium comprising ascorbic acid, preferably the serum-free medium is a CGMP-compliant medium.

In a preferred embodiment, step c) includes the steps of
freezing the generated precursor-HSPC-pDCs after priming; or
freezing, storing and thawing the generated precursor-HSPC-pDCs before priming; or
freezing, storing and thawing the generated precursor-HSPC-pDCs after priming.

As shown in examples 11 and 12, freezing (cryopreservation) is possible before or after priming, while preserving function.

In another aspect, the present invention relates to a process for producing HSPC-derived Plasmacytoid dendritic cells (HSPC-pDCs) from hematopoietic stem and progenitor cells (HSPCs), the process comprising the steps:
a) providing hematopoietic stem and progenitor cells (HSPCs);
b) differentiating said HSPCs, to generate precursor-HSPC-pDCs;
c) priming said precursor-HSPC-pDCs with interferon to provide mature HSPCp-DCs; and
d) activating the mature HSPC-pDCs to induce secretion of one or more cytokines, such as type I and/or III interferon,
   wherein steps b)-d) are carried out in serum free medium comprising ascorbic acid, preferably the serum-free medium is a CGMP-compliant medium.

In a preferred embodiment, the process further comprising the step:
d) activating the mature HSPC-pDCs to induce secretion of type I interferon;
   wherein step d) are carried out in serum free medium comprising ascorbic acid, preferably the serum-free medium is a CGMP-compliant medium.

In another preferred embodiment, the process further comprising the step:
d) activating the mature HSPC-pDCs to induce secretion of one or more cytokines, such as type I and/or III interferon;
   wherein step d) are carried out in serum free medium comprising ascorbic acid, preferably the serum-free medium is a CGMP-compliant medium.

During the steps b)-d) different types of growth media can be used known to the skilled person. Types of growth media includes non-CGMP medium, such as RPMI 1640 supplemented with fetal calf serum (FCS) or human serum, commercially-available serum-free medium, such as StemSpan™ SFEM II, or CGMP compliant medium, such as StemSpan™-ACF, CellGenix@ GMP SCGM, or CellGenix@ GMP DC Medium, supplemented with ascorbic acid. Preferably a CGMP-compliant medium supplemented with ascorbic acid should be used.

In in one embodiment, the media is commercially-available serum free media In another embodiment, the media is commercially-available growth media supplemented with serum.

In a preferred embodiment, the media is serum free CGMP compliant medium.

Step a) Provision of HSPCs

HSPCs can be supplied from different sources. The cells are found in bone marrow, peripheral blood or umbilical cord blood.

Thus, in one preferred embodiment, the process according to the present invention, wherein in step a), the HSPCs are provided from circulating HSPCs (cHSPC) e.g. found in peripheral blood.

In addition, umbilical cord blood is blood that remains in the placenta and in the attached umbilical cord after child birth.

Thus, in another preferred embodiment, the provided HSPCs in step a) are derived from umbilical cord blood (UCB).

In a further embodiment, the provided HSPCs in step a) are derived from bone marrow.

Similarly, in certain embodiments, step a) comprises providing a peripheral blood sample or an umbilical cord blood sample that comprises hematopoietic stem and progenitor cells (HSPCs).

In preferred embodiments, step a) comprises providing HSPCs or a sample comprising HSPCs that have previously been obtained from a subject. Step a) does not encompass obtaining HSPCs or a sample from a subject.

In another embodiment, the blood is mammalian blood, such as animal or human blood.

In another preferred embodiment, the provided HSPCs in step a) are from mobilized peripheral blood (mPB HSPCs) were donors undergo mobilization of HSPCs by injection of mobilization agent, such as granulocyte-colony stimulating factor (GM-CSF).

In a preferred embodiment, the blood is human blood.

In yet another embodiment, the cells are mammalian cells, such as animal or human cells.

In a preferred embodiment, the cells are human cells.

To be able to distinguish the different cells types in the human body, cells are characterized by their expression of surface markers. CD34 is found on haematopoietic cells.

Thus, in an embodiment, the provided HSPCs in step a) are CD34+ cells.

After the HSPCs are obtained they can either be freshly applied to the procedure according to the invention or the cells can be cryopreserved for later use.

Thus, in one embodiment according to the invention, the provided HSPCs in step a) are fresh cells or cryopreserved cells.

During steady state in the body of the subject, most HSPCs are located in the bone marrow whereas very few are found in the peripheral blood. Treatment with G-CSF or plerixafor can enforce HSPC mobilization to the peripheral blood. To date, G-CSF mobilized HSPCs widely used for transplantation but has several limitations such as the need of a HLA match between donor and recipient. Further the method requires multiple injections of G-CSF usually over four consecutive days followed by apheresis and large-scale CD34 immunomagnetic selection. Thus, the method is time-consuming, costly, requires access to expensive equipment and is associated with inconvenience to the donor and side effects such as bone pain.

Thus, in an embodiment, the HSPCs provided in step a) are provided from a subject without a prior mobilization regiment of the HSPCs in said subject, such as by mobilization by G-CSF and/or plerixafor.

In another embodiment the HSPCs provided in step a) are provided from a subject without a prior mobilization regiment of the HSPCs in said subject, such as by mobilization by G-CSF.

In yet another embodiment the HSPCs provided in step a) are provided from a subject without a prior mobilization regiment of the HSPCs in said subject, such as by mobilization by plerixafor.

As described above, drawbacks are related to mobilization regiment of HSPCs in the subject. Nevertheless, the process according to the invention can still be carried out in a subject exposed to the mobilization regiment of HSPCs.

Thus, in one embodiment, the HSPCs provided in step a) are provided from a subject exposed to mobilization of the HSPCs, such as mobilization by G-CSF and/or plerixafor.

Step b) Differentiation

Culturing HSPCs ex vivo at low density culture conditions, before initiating the differentiation, stimulates the transition of HSPCs into the cell cycle, thereby supporting the expansion.

As seen in example 2 when the cells were kept at a density below 5e+6 cell/ml the expansion rate was more than 27× higher compared to cells not cultured at low density conditions. Further the low density protocol led to an increase in the HSPC-pDCs as well.

Thus, in one embodiment step b) of the process according to the invention comprises the step b1) and step b2) comprising:
b1) pre-expanding the hematopoietic stem and progenitor cells (HSPCs) provided in step a) starting with a concentration of 0.1-0.5×10⁶ cells/mL for up to 8 days; and
b2) differentiating the pre-expanded cells from step b1) to generate precursor-pDCs.

A fundamental limitation to HSPC ex vivo culturing is the rapid differentiation of the stem and progenitor cells, which in turn produces inhibitory feedback signals that limits stem cell self-renewal. Supplying the ex vivo culturing of HSPCs with the small molecules UM171 and SR1 promotes self-renewal of primitive hematopoietic progenitor cells.

In an embodiment, step b) is performed in the presence of one or more small molecule inhibitors, such as UM171 and/or StemRegenin 1, preferably in the presence of UM171 and/or StemRegenin 1.

In another embodiment, the concentration of StemRegenin 1 is in the range 0.05-5 μM, such as 0.25-2 μM, such as 0.5-1.5 μM, or such as 0.75-1.25 μM. In a preferred embodiment, the concentration of StemRegening is around 1 μM.

In another embodiment, the concentration of UM171 is in the range 3-100 nM such as in the range 10-70 nM, such as in the range 10-50 nM, such as 20-40 nM. In a preferred embodiment, the concentration of UM171 is around 35 nM.

In an embodiment, step b) is performed in is performed in the presence of an aryl hydrocarbon receptor antagonist, such as SR1.

In another embodiment, step b) is performed in in the presence of IL-3. In a related embodiment, the concentration of IL-3 is in the range 1-200 ng/mL, such as the range 1-100 ng/mL, such as 1-50 ng/mL, preferably in the range of 10-20 ng/mL, such as 20 ng/mL of IL-3.

In another embodiment, in step b1), cell density is kept in the range 0.1-50×10⁵ cells/mL, such as in the range 0.5-20×10⁵ cells/mL, preferably in the range 1-5×10⁵ cells/mL, such as in the range 5-50×10⁵.

In a preferred embodiment, in step b1), cell density is kept in the range of 1-5×10⁵ cell/ml, such as below 5×10⁵ cell/ml.

In a further embodiment in step b2), cell density is kept in the range 0.1-50×10⁵ cells/mL, such as in the range 0.5-20×10⁵ cells/mL, preferably in the range 1-5×10⁵ cells/mL, such as in the range 5-50×10⁵.

In a preferred embodiment, in step b2), cell density is kept in the range of 5-50e+5 cell/ml such as below 50+e5 cell/ml.

In yet a further embodiment, step b1) is continued for up to 8 days, such as up to 6 days, such as up to 4 days, preferably 4 days.

In one embodiment, step b2) is performed for up to 21 days of culture, such as up to 18 days, preferably up to 16 days of culture.

As seen in example 2 starting with 2×10⁵ HSPCs, the low density protocol led the cells expand up to 1.5×10⁹ total cells, compared to the conventional protocol only reaching 0.055×10⁹ of total cells. This an improvement of more than 27-fold over the standard condition (see example 2).

Thus, in an embodiment, the hematopoietic stem and progenitor cells (HSPCs) in step b1) are expanded at least 10 times, such as at least 15 times, such as at least 20 times, or such as at least 25 times.

Step c) Priming of the HSPC-pDCs

For isolated HSPC-pDCs to be fully functional, the cells require priming by type I or II IFN added to the culture medium. The culture medium may further supplemented with penicillin and streptomycin (P/S) to avoid microbiological infections.

In an embodiment in priming step c), the priming medium comprises P/S or IL-3.

In another embodiment, the concentration of penicillin is in the range 2-100 U/ml, such as in the range 2-50 U/ml, such as in the range 5-30 U/ml, such as in the range 10-30 U/ml, or such as in the range 15-25 U/ml. In a preferred embodiment, the concentration of penicillin is around 20 U/ml.

In another embodiment, the concentration of streptomycin is in the range 2-100 μg/ml, such as in the range 2-50 μg/ml, such as in the range 5-30 μg/ml, such as in the range 10-30 μg/ml, or such as in the range 15-15 μg/ml. In a preferred embodiment, the concentration of streptomycin is around 20 μg/ml.

In another embodiment the concentration of IL-3 is in the range 2-100 ng/ml, such as in the range 2-50 ng/ml, such as in the range 5-30 ng/ml, such as in the range 10-30 ng/ml, or such as in the range 15-15 ng/ml. In a preferred embodiment, the concentration of IL-3 is around 20 ng/ml.

During the pre-expansion (step b) the cells were cultured in medium supplemented with growth factors, such as Flt3-L, TPO and SCF, together with small-molecule inhibitors SR1 and UM171.

During priming the media should preferably be free of these factors, to promote the maturation and priming of pDCs.

Thus, in an embodiment in priming step c), the priming medium is free of growth factors different from P/S and IL-3, such as being free of at least Flt3-L, TPO, and SCF, and small-molecule inhibitors SR1 and UM171.

In an embodiment in priming step c), said priming medium comprises type I and/or type II IFNs, such as comprising subtypes of IFN-α and/or IFN-β and/or IFN-γ, preferably comprising both IFN-β and IFN-γ.

In another embodiment priming step c) is performed for up to 5 days, preferably up to 3 days, such as 1-3 days or 2-3 days.

In a preferred embodiment, the priming step c) is performed for 3 days.

As outlined in example 11 and 12, cryopreservation in step c), before or after priming, is possible. Thus, in a preferred embodiment, step c) includes the steps of
freezing the generated precursor-HSPC-pDCs after priming; or
freezing, storing and thawing the generated precursor-HSPC-pDCs before priming; or
freezing, storing and thawing the generated precursor-HSPC-pDCs after priming.

In an embodiment, freezing is conducted by cryopreservation, such as by lowering the temperature to a temperature in the range −80° C. to −196° C.

In another embodiment, freezing is conducted in a cryopreservation medium preferably, serum-free medium, preferably free from animal components, preferably cGMP-manufactured, such as CryoStor CS10.

In yet an embodiment, storage is conducted and temperatures below −4° C., such as below −10° C., preferably below −15°, more preferably at −20° C. or lower, such as at −70° C. or lower, such as in the range −80° C. to −196° C. or such as in liquid nitrogen.

In a related embodiment, storage is conducted from 5 hours to 1 year, such as 1 day to 6 month, such as 7 days to 2 month.

In an embodiment, thawing is conducted using serum-free medium, such as CellGenix DC medium, CellGenix SCGM or SFEM I or II, preferable CellGenix DC medium.

In one embodiment, freezing is conducted before priming.

In another preferred embodiment, freezing is conducted after priming. In such a case are “ready-to-use” product is produced. This will allow for production at one dedicated facility, followed by shipment to the location of use, e.g. a hospital facility.

Step d)—Activation

For the HSPC-pDCs to be fully functional active, the cells are exposed to stimulatory molecules leading to activation of the cells.

Thus, in an embodiment of the present invention, step d) is performed in the presence of agonists, such as a TLR7/8 agonist and/or a TLR9 agonist, and/or a STING agonist and/or RIG-I agonist, and/or viral agonist such as Influenza A or Herpes simplex virus (HSV), preferably in the presence of a TLR7/8 agonist and/or a TLR9 agonist.

In another embodiment of the present invention, step d), is performed in the presence of an antigen, such as a tumor-associated antigen or a viral antigen in the presence of a TLR7 agonist and/or a TLR9 agonist, and/or a STING agonist and/or RIG-I agonist, and/or viral agonist such as Influenza A, Tick-borne encephalitis virus (TBEV), or Herpes simplex virus (HSV), preferably in the presence of TLR7 agonist and TLR9 agonist.

In a preferred embodiment, step d) is performed in the presence of an agonist such as a TLR7/8 agonist and/or a TLR9 agonist.

In yet an embodiment, step d) is performed
in the presence of an antigen, such as a tumor-associated antigen or a viral antigen in the presence of a TLR7/8 agonist and/or a TLR9 agonist, and/or a STING agonist and/or RIG-I agonist, and/or viral agonist such as Influenza A, Tick-borne encephalitis virus (TBEV), or Herpes simplex virus (HSV); preferably a tumor-associated antigen in the presence of TLR7 agonist and TLR9 agonist;
OR
in the presence of a TLR7/8 agonist and/or a TLR9 agonist, and/or a STING agonist and/or RIG-I agonist, and/or viral agonist such as Influenza A, Tick-borne encephalitis virus (TBEV), or Herpes simplex virus (HSV), preferably TLR7/8 agonist and/or TLR9 agonist.

In another embodiment, in activation step d), said activation medium is free of growth factors different from P/S and IL-3, such as being free of at least Flt3-L, TPO and SCF and small-molecule inhibitors SR1 and UM171.

In a further embodiment of the present invention, activation step d), is performed in the presence of a tolerogenic modifying compound, such as corticosteroid dexamethasone, cyclosporine or acetylsalicylic acid, IL-10 or TGF-beta, preferably in the presence of IL-10 or TGF-beta

As mentioned above, ascorbic acid is added to the growth media during the process according to the invention. Ascorbic acid (vitamin C) is an essential vitamin in humans known to have pleiotropic functions in cellular biology, including immune cell function and haematopoiesis. Further ascorbic acid is involved in type I IFN immune responses.

In one embodiment, the media of the present invention is supplemented with ascorbic acid.

In yet an embodiment step b- and step c) are performed in the presence of 10-200 μg/mL of ascorbic acid, such as in the range 10-150 μg/mL, such as in the range 10-100 μg/mL, preferably in the range 25-75 μg/mL, more preferably in the range 35-65 μg/mL, or such as around 50 μg/mL of ascorbic acid; In another embodiment, step b) to step d) are performed in the presence of 10-200 μg/mL of ascorbic acid, such as in the range 10-150 μg/mL, such as in the range 10-100 μg/mL, preferably in the range 25-75 μg/mL, more preferably in the range 35-65 μg/mL, or such as around 50 μg/mL of ascorbic acid.

In another embodiment, ascorbic acid is added to the media in concentration of 10 μg/ml, 20 μg/ml, 30 μg/ml, 40 μg/ml, 50 μg/ml, 60 μg/ml, 70 μg/ml, 80 μg/ml, 90 μg/ml or 100 μg/ml. In a preferred embodiment, ascorbic acid is added to the media in a concentration of 50 μg/ml.

In another preferred embodiment, ascorbic acid was added to the media in physiological concentrations.

In another embodiment, the physiological concentration is human physiological concentrations.

HSPC-pDCs Obtained/Obtainable by the Process of the Invention

An aspect of the invention relates to the HSPC-pDCs obtained/obtainable by a process according to the invention. Further, as seen in examples 8 and 9 the HSPC-pDCs according to the invention exhibits a unique and novel RNA expression profile enabling the skilled person to distinguish the cells from other pDCs.

Preferably the HSPC-pDCs are cryopreserved or have been cryopreserved, such as after differentiation.

TLR7 and TLR9 pathway-related genes may be particularly relevant to have expression of. Thus, in an embodiment the HSPC-pDCs according to the invention, express one or more genes selected from the group consisting of genes in table 3 (see example 9).

In an embodiment the HSPC-pDCs express (or express an increased levels of) at least 5 such as at least 10, such as at least 20, such as at least 30, such as at least 40 or such as all of the genes listed above or in Table 3.

In a preferred embodiment, the one or more genes are selected from the group consisting of AP3S2, CLEC4C, FCER1G, IRF7, IRF8, LAMP5, LILRA4, MYD88, NRP1, PACSIN1, PLSCR1, TLR7, TLR8, TLR9, TRAF3, UBE2N, and UNC93B1. These TLR7 and TLR9 pathway-related genes are significantly differentially expressed genes (all upregulated) upon generating HSPC-pDCs in ascorbic acid-containing medium (see example 9).

In yet an embodiment, the HSPC-pDCs according to the invention
express one or more genes selected from the group consisting of AP3S2, CLEC4C, FCER1G, IRF7, IRF8, LAMP5, LILRA4, MYD88, NRP1, PACSIN1, PLSCR1, TLR7, TLR8, TLR9, TRAF3, UBE2N, and UNC93B1; and/or
express an increased level of one or more genes selected from the group consisting of AP3S2, CLEC4C, FCER1G, IRF7, IRF8, LAMP5, LILRA4, MYD88, NRP1, PACSIN1, PLSCR1, TLR7, TLR8, TLR9, TRAF3, UBE2N, and UNC93B1 compared to HSPC-pDC isolated from blood; and/or
express an increased level of one or more genes selected from the group consisting of AP3S2, CLEC4C, FCER1G, IRF7, IRF8, LAMP5, LILRA4, MYD88, NRP1, PACSIN1, PLSCR1, TLR7, TLR8, TLR9, TRAF3, UBE2N, and UNC93B1 compared to HSPC-pDCs produced from hematopoietic stem and progenitor cells (HSPCs), in the absence of ascorbic acid; and/or
express an increased level of the one or more genes selected from the group consisting of AP3S2, CLEC4C, FCER1G, IRF7, IRF8, LAMP5, LILRA4, MYD88, NRP1, PACSIN1, PLSCR1, TLR7, TLR8, TLR9, TRAF3, UBE2N, and UNC93B1 compared to HSPC-pDCs produced in the absence of ascorbic acid in step b) or c) or d), such as compared to HSPC-pDCs produced in absence of ascorbic acid in steps b)-d).

In an embodiment the HSPC-pDCs express (or express an increased levels of) at least 5 such as at least 7, such as at least 9, such as at least 11, such as at least 13 or such as all of the genes of AP3S2, CLEC4C, FCER1G, IRF7, IRF8, LAMP5, LILRA4, MYD88, NRP1, PACSIN1, PLSCR1, TLR7, TLR8, TLR9, TRAF3, UBE2N, and UNC93B1.

In a preferred embodiment the HSPC-pDCs express (or express an increased levels of) at least one of IRF7, IRF8, MYD88, NRP1, TLR7, TLR8, TLR9, and UNC93B1, such as at least three, such as at least five or such as all of IRF7, IRF8, MYD88, NRP1, TLR7, TLR8, TLR9, and UNC93B1.

As previously described, different subtypes of DC are present in the immune system. Conventional DCs (cDC) are the most common, whereas pDCs represent a more rare and unique type. One way of distinguishing the different types of cells is by the expression of surface markers.

cDCs are characterized by high expression of CD11c whereas pDCs are characterized by low expression of CD11c combined with high expression of both CD123 and CD303. Further both cDCs and pDCs are characterized by lacking expression of markers for other cell lineages (Lin⁻), such as T cells, monocytes, B cells, macrophages, granulocytes etc.

Thus, in an embodiment, the pDCs according to the invention, has the phenotype Lin⁻/CD11c⁻/CD123⁺/CD303⁺.

The specific process of the invention also influences the overall expression profile of the cells. Thus, in an embodiment, the HSPC-pDCs according to the invention, express one or more genes selected group consisting of CD74, FTH1, HLA-DRB1, HLA-DPA1, IFI™3, FCER1G, S100A11, DEFA1, PSAP, DEFA4, CTSD, GRN, ITGB2, CD68, DEFA3, TYMP, CHI3L1, SERPING1, CTSZ, RETN, HLA-DQA1, HLA-DPB1, IFI27, HLA-DRB3, C1QC, ALOX5AP, CTSB, BRI3, ANXA2, C1QB, CYBB, LGALS3BP, HLA-DMB, SOD2, CTSH, C1orf162, CTSS, EVI2B, CD81, C1QA, PRDX1, APP, GRINA, MX1, IL2RG, NCF1, FLNA, LGALS3, and ADA2. These genes have been identified as upregulated in the pDCs according to the invention (see example 8, table 1).

In an embodiment the HSPC-pDCs express (or express an increased levels of) at least 5 such as at least 10, such as at least 20, such as at least 30, such as at least 40 or such as all of the genes listed above or in Table 1.

In yet an embodiment, the HSPC-pDCs express (or express an increased levels of) at least one of HLA-DRB1, HLA-DPA1, HLA-DQA1, HLA-DPB1, HLA-DRB3, and HLA-DMB, such as at least 3, such as at least five or such as all of HLA-DRB1, HLA-DPA1, HLA-DQA1, HLA-DPB1, HLA-DRB3, and HLA-DMB. These are considered MHC-II genes.

In yet an embodiment, the HSPC-pDCs according to the invention
express one or more genes from the group of genes listed in Table 1 and/or Table 2 and/or Table 3; and/or
express an increased level of one or more genes from the group of genes listed in Table 1 and/or Table 2 and/or Table 3 compared to HSPC-pDC isolated from blood; and/or
express an increased level of one or more genes from the group of genes listed in Table 1 and/or Table 2 and/or Table 3 compared to HSPC-pDCs produced from hematopoietic stem and progenitor cells (HSPCs), in the absence of ascorbic acid; and/or
express an increased level of one or more genes from the group of genes listed in Table 1 and/or Table 2 and/or Table 3 compared to HSPC-pDCs produced in the absence of ascorbic acid in step b) or c) or d), such as compared to HSPC-pDCs produced in absence of ascorbic acid in steps b)-d).

In another embodiment, the HSPC-pDCs according to the invention, express one or more genes selected from the group consisting of genes in Table 2 (see example 8).

In yet an embodiment the HSPC-pDCs express (or express an increased level of) at least 5 such as at least 10, such as at least 20, such as at least 30, such as at least 40 or such as all of the genes listed above or in Table 2.

In a preferred embodiment, the HSPC-pDCs express (or express an increased levels of) at least one of AXL and TLR7 or at least AXL and TLR7.

Thus, the cells according to the invention indeed have a unique cell expression profile compared to known HSPC-pDCs.

Following activation according to the invention, the HSPC-pDCs starts the production of cytokines and chemokines.

In one embodiment of the present invention, the activated HSPC-pDCs produce interferons, such as type I and/or type II and/or type III interferons, IL1-beta, IL-6, IL-8, TNF-alpha and/or ligands for CXCR3 such as CXCL9, CXCL10 and CXCL11.

In another embodiment, the HSPC-pDCs produce interferons (IFN).

In a preferred embodiment, the HSPC-pDCs produce Type I IFN following activation.

In another preferred embodiment, the HSPC-pDCs produce Type II IFN following activation.

The amount of cytokines and chemokines, such as IFNs produced following activation depends on different factors. If the cells are primed (step c) with Type I and II interferon according to the invention before activation, pDCs will after stimulation with TLR7 agonist produce type I interferon in a range of 500-4000 U/ml. Stimulation with TLR9 agonist will following priming lead to a type I interferon production of 1000-10.000 U/ml.

In an embodiment, the HSPC-pDCs produces the above-mentioned cytokines and chemokines before activation of the cells.

In a further embodiment, the HSPC-pDCs produce the above-mentioned cytokines and chemokines without being primed.

In further embodiments, the invention provides a population of HSPC-derived pDCs that comprises at least 1.7 million pDCs, such as at least 2 million, 5 million, 10 million, 15 million, 20 million, 25 million or 30 million pDCs.

HSPC-pDCs

As also outlined above, the HSPC-pDCs produced according to the invention exhibit a unique and novel RNA expression profile (see examples 8 and 9). In particular, TLR7 and TLR9 pathway-related genes may be particular relevant to have expression of. Thus an aspect of the invention relates to isolated HSPC-pDC cells, which
express one or more genes selected from the group consisting of AP3S2, CLEC4C, FCER1G, IRF7, IRF8, LAMP5, LILRA4, MYD88, NRP1, PACSIN1, PLSCR1, TLR7, TLR8, TLR9, TRAF3, UBE2N, and UNC93B1; and/or
express an increased level of one or more genes selected from the group consisting of AP3S2, CLEC4C, FCER1G, IRF7, IRF8, LAMP5, LILRA4, MYD88, NRP1, PACSIN1, PLSCR1, TLR7, TLR8, TLR9, TRAF3, UBE2N, and UNC93B1 compared to HSPC-pDC isolated from blood; and/or
express an increased level of one or more genes selected from the group consisting of AP3S2, CLEC4C, FCER1G, IRF7, IRF8, LAMP5, LILRA4, MYD88, NRP1, PACSIN1, PLSCR1, TLR7, TLR8, TLR9, TRAF3, UBE2N, and UNC93B1 compared to HSPC-pDCs produced from hematopoietic stem and progenitor cells (HSPCs), in the absence of ascorbic acid; and/or
express an increased level of the one or more genes selected from the group consisting of AP3S2, CLEC4C, FCER1G, IRF7, IRF8, LAMP5, LILRA4, MYD88, NRP1, PACSIN1, PLSCR1, TLR7, TLR8, TLR9, TRAF3, UBE2N, and UNC93B1 compared to HSPC-pDCs produced in the absence of ascorbic acid in step b) or c) or d), such as compared to HSPC-pDCs produced in absence of ascorbic acid in steps b)-d).

Preferably the HSPC-pDCs are cryopreserved or have been cryopreserved, such as after differentiation.

In a related embodiment, the HSPC-pDCs express (or express an increased levels of) at least 5 such as at least 7, such as at least 9, such as at least 11, such as at least 13 or such as all of the genes of AP3S2, CLEC4C, FCER1G, IRF7, IRF8, LAMP5, LILRA4, MYD88, NRP1, PACSIN1, PLSCR1, TLR7, TLR8, TLR9, TRAF3, UBE2N, and UNC93B1. Again, as also outlined in example 9, these genes are considered TLR7 and TLR9 pathway-related genes.

In yet an aspect, the HSPC-pDCs according to the invention
express one or more genes from the group of genes listed in Table 1 and/or Table 2 and/or Table 3; and/or
express an increased level of one or more genes from the group of genes listed in Table 1 and/or Table 2 and/or Table 3 compared to HSPC-pDC isolated from blood; and/or
express an increased level of one or more genes from the group of genes listed in Table 1 and/or Table 2 and/or Table 3 compared to HSPC-pDCs produced from hematopoietic stem and progenitor cells (HSPCs), in the absence of ascorbic acid; and/or
express an increased level of one or more genes from the group of genes listed in Table 1 and/or Table 2 and/or Table 3 compared to HSPC-pDCs produced in the absence of ascorbic acid in step b) or c) or d), such as compared to HSPC-pDCs produced in absence of ascorbic acid in steps b)-d).

In an embodiment the HSPC-pDCs express (or express an increased levels of) at least 5 such as at least 10, such as at least 20, such as at least 30, such as at least 40 or such as all of the genes listed above or in Table 1, Table 2 or Table 3.

In a preferred embodiment, the HSPC-pDCs express (or express an increased levels of) at least one of AXL and TLR7 or at least AXL and TLR7.

In another preferred embodiment, the HSPC-pDCs express (or express an increased levels of) at least one of HLA-DRB1, HLA-DPA1, HLA-DQA1, HLA-DPB1, HLA-DRB3, and HLA-DMB, such as at least 3, such as at least five or such as all of HLA-DRB1, HLA-DPA1, HLA-DQA1, HLA-DPB1, HLA-DRB3, and HLA-DMB. These are considered MHC-II genes.

Again, as outlined in example 8 and 9 (see also Tables 1, 2 and 3), cells produced according to the invention have a unique expression profile.

It is of course to be understood that embodiments from aspect may be combined with the other aspect of this invention. Thus, e.g. embodiments relating to “products obtained by the process of the invention (“product-by-process”) can also be combined with aspects relating to the HSPC-pDCs according to the invention.

Medical Use

The HSPC-pDCs produced according to the invention, can be used as a medicament for treating different diseases.

Thus, in an embodiment the HSPC-pDCs according to the invention are used as a medicament.

In another embodiment, the HSPC-pDCs according to the invention is for use in the treatment or alleviation of cancer.

In yet another embodiment, said cancer is selected from brain cancer, glioblastoma, lung cancer, colorectal cancer, skin cancer, malignant melanoma, pancreas cancer, bladder cancer, liver cancer, breast cancer, eye cancer and prostate cancer.

In yet another embodiment, said cancer is a haematological cancer, such as selected from the group consisting of multiple myeloma, acute myeloblastic leukemia, chronic myelogenic leukemia, acute lymphoblastic leukemia and chronic lymphocytic leukemia.

In a preferred embodiment, said cancer is malignant melanoma, breast cancer, None-small cell lung cancer, pancreatic cancer, head&neck cancer, liver cancer, sarcoma, or B cell lymphoma.

A non-limiting way for using the HSPC-pDCs of the invention, in treatment of cancer is by antigen loading of the cells. This method is possible, since HSPC-pDCs are the most potent antigen representing cells and are essential for initiating primary immune responses. By exposing the HSPC-pDCs to an antigen derived from the cells of interest, the cells can take up the antigen and start presenting it to the surroundings. Thereby the cells are fully ready to activate the immune system of the subject, when initiated as a vaccine.

Uses

Another aspect of the present invention relates to the use of ascorbic acid in a CGMP serum-free medium for promoting viability and expansion of HSPCs, and for promoting the development and functionality of HSPC-pDCs, such as the secretion of type I IFN following activation.

A further aspect of the present invention related to the use of ascorbic acid in CGMP medium for inducing cytokine and chemokine secretion in HSPC-pDCs.

In an aspect the use of ascorbic acid in a CGMP serum-free medium is for inducing expression of TLR7 and TLR9 pathway-related genes, such as selected from the group consisting of AP3S2, CLEC4C, FCER1G, IRF7, IRF8, LAMP5, LILRA4, MYD88, NRP1, PACSIN1, PLSCR1, TLR7, TLR8, TLR9, TRAF3, UBE2N, and UNC93B1.

In another aspect the use of ascorbic acid in a CGMP serum-free medium is for inducing expression of one or more of the genes listed in Table 1 and/or Table 2 and/or Table 3.

It should be noted that embodiments and features described in the context of one of the aspects of the present invention also apply to the other aspects of the invention.

In another embodiment, the HSPC-pDCs according to the invention is for use in the treatment or alleviation of autoimmune disease, and transplant-rejection.

In yet another embodiment, said autoimmune disease is celiac disease, inflammatory bowel disease, Graves' disease, multiple sclerosis, psoriasis, rheumatoid arthritis, and systemic lupus erythematosus.

In a preferred embodiment, said autoimmune disease is systemic lupus erythematosus, rheumatoid arthritis, multiple sclerosis and psoriasis.

In yet another embodiment, said transplant-rejection includes mild, moderate or severe graft-versus-host disease.

In a preferred embodiment, said transplant-rejection is mild, moderate or severe graft-versus-host disease.

A non-limiting way for using the HSPC-pDCs of the invention, in treatment of autoimmune diseases or transplantation rejection is by antigen loading the cells and simultaneously inducing a tolerogenic phenotype. This method is possible by activating pDCs under strict conditions to obtain a tolerogenic phenotype. By exposing the HSPC-pDCs to antigen in combination with a tolerogenic stimuli, such as corticosteriod dexamethasone, cyclosporine, acetylsalicylic acid, IL10, or TGF-beta, a tolerogenic phenotype will be induced by the HSPC-pDCs. The HSPC-pDCs will take up the antigen and start presenting it to the surroundings. Thereby the cells are fully ready to regulate the immune system of the subject, when initiated as a vaccine.

In another embodiment, the HSPC-pDCs according to the invention is for use in the treatment or alleviation or infectious diseases.

In yet another embodiment, said infectious diseases include coronaviruses, such as SARS-CoV-2, Ebola, Influenza, Human immunodeficiency virus (HIV), Hepatitis, and Zika virus.

In a preferred embodiment, said infectious diseases include coronaviruses, Influenza and HIV.

A non-limiting way for using the HSPC-pDCs of the invention, in treatment of infectious diseases is by antigen loading of the cells. This method is possible, since HSPC-pDCs are the most potent antigen representing cells and are essential for initiating primary immune responses. By exposing the HSPC-pDCs to an antigen derived from the cells of interest, the cells can take up the antigen and start presenting it to the surroundings. Thereby the cells are fully ready to activate the immune system of the subject, when initiated as a vaccine.

Methods of Preparing Therapeutic Compositions

In further embodiments of the invention, there is provided a method of preparing a therapeutic composition comprising:
a) providing hematopoietic stem and progenitor cells (HSPCs);
b) differentiating said HSPCs, to generate precursor-HSPC-derived-pDCs;
c) priming said precursor-HSPC-derived-pDCs with interferon to provide mature HSPC-derived-pDCs;
d) optionally activating the mature HSPC-derived-pDCs to induce secretion of type I interferon;
e) optionally loading the mature HSPC-derived-pDCs with antigens, such as tumour antigens, or transforming the mature HSPC-derived pDCs with an exogenous construct, such as a CAR-T construct; and
f) formulating said mature HSPC-derived-pDCs into a therapeutic composition,
   wherein, steps b) c) and d) are carried out in serum-free medium comprising ascorbic acid and preferably the serum-free medium is a CGMP-compliant medium.

All patent and non-patent references cited in the present application, are hereby incorporated by reference in their entirety.

The invention will now be described in further details in the following non-limiting examples.

### EXAMPLES

### Example 1—Materials and Methods

Generation of HSPC-pDCs from CD34+HSPCs

Depending on optimization regiments, freshly or cryo-preserved HSPCs from various sources were differentiated to HSPC-pDCs using different types of medium. For STD and LD/RPMI conditions, HSPCs were cultured in RPMI 1640 (Lonza) supplemented with 10% heat-inactivated fetal calf serum (FCS) (HyClone®), 600 μg/mL L-Glutamine (Sigma), 200 U/mL penicillin and 100 μg/mL streptomycin (Gibco®, Life Technologies). For STD conditions, HSPCs were cultured at a fixed volume during pDC differentiation, versus a fixed density of 0.5-5×10⁶ cells/mL for the LD condition. For serum-free (SFEM II) or CGMP serum-free medium (DC medium) conditions, HSPCs were cultured in SFEM II or DC medium at low density (0.5-5×10⁶ cells/mL). For all conditions, medium was supplemented was supplemented with the cytokines and growth factors Flt3-L (100 ng/mL), SCG (100 ng/mL), TPO (50 ng/mL), IL-3 (20 ng/mL). In contrast to our previous protocol, we omitted the addition of IL-7 as we showed that this did not positively impact HSPC-pDC generation [10]. All cytokines are from Peprotech. In addition, the small molecule inhibitor StemRegenin 1 (SR1, STEMCELL Technologies) was added at a concentration of 1 μM. For serum-free medium conditions, a concentration of 20 μg/mL streptomycin and 20 U/mL penicillin (Gibco, Life Technologies) was added. Moreover, for the CGMP DC medium condition, 50 μg/mL of ascorbic acid was added. Cells were cultured at 37° C., 95% humidity, and 5% CO₂ for up to 21 days depending on optimizations. For the fixed density conditions, medium was replenished every 2-4 day depending on the growth of the HSPCs. Total cell numbers during expansion and differentiation of HSPCs was determined using a TC20 Automated Cell Counter (Bio-Rad). At the end of culture, HSPC-pDCs were enriched using a negative selection kit, according to manufacturer's instructions (EasySep Human Plasmacytoid Dendritic Cell Enrichment kit, STEMCELL Technologies).

Isolation of HSPC from Peripheral Blood

CD34+HSPC were purified from healthy blood donors using the EasySep Complete Kit for Human Whole Blood CD34+Cells, according to manufacturer's instructions (STEMCELL Technologies). Briefly, a pre-enrichment of CD34+HSPCs was performed were bi-specific antibodies targeting unwanted cells were used during standard Ficoll-Hypaque (GE Healthcare) density-gradient centrifugation. CD34+ cells were subsequently isolated using anti-CD34 immunomagnetic beads (positive selection). CD34+cHSPCs were either freshly used or cryo-preserved until use.

Isolation of HSPC from Cord Blood

De-identified umbilical cord blood (UCB) samples were obtained following scheduled caesarean section deliveries of healthy infants at Department of Gynecology and Obstetrics, Skejby University Hospital. Consent was obtained from the mothers. CD34⁺ cord blood HSPCs (CB-HSPC) were subsequently purified using EasySep Human Cord Blood CD34 Positive Selection kit II, according to manufacturer's instructions (STEMCELL Technologies). Briefly, a pre-enrichment of CD34+ cells was performed were bi-specific antibodies targeting unwanted cells were used during standard Ficoll-Hypaque (GE Healthcare) density-gradient centrifugation. CD34+ cells were subsequently isolated using anti-CD34 immunomagnetic beads (positive selection). CD34⁺ CB HSPCs were either freshly used or cryo-preserved until use.

Isolation of cHSPC from Peripheral Blood

Buffy coat samples were obtained from normal healthy donors from Aarhus University Hospital Blood Bank. CD34+cHSPCs were subsequently purified using the EasySep Complete Kit for Human Whole Blood CD34+Cells, according to manufacturer's instructions (STEMCELL Technologies). Briefly, a pre-enrichment of CD34+ cells was performed were bi-specific antibodies targeting unwanted cells were used during standard Ficoll-Hypaque (GE Healthcare) density-gradient centrifugation. CD34⁺ cells were subsequently isolated using anti-CD34 immunomagnetic beads (positive selection). CD34⁺ cHSPCs were cryo-preserved until use.

Pre-Expansion of CB-HSPC or cHSPC

In order to pre-expand HSPCs, cells was seeded at low density (1×10⁵ cells/mL) in SFEM II (STEMCELL Technologies) or SCGM (CellGenix) to enable non-CGMP or CGMP conditions, respectively. Medium was supplemented 20 μg/mL streptomycin and 20 U/mL penicillin (Gibco, Life Technologies), as well as 100 ng/mL of the growth factors Flt3-L, TPO and SCF (Peprotech). In addition, medium was supplemented with the small molecule inhibitors, StemRegenin 1 (1 μM, SR1 STEMCELL Technologies) and UM171 (35 nM, STEMCELL Technologies). Throughout the period of pre-expansion, cells were kept at low density (1-5×10⁵ cells/mL), and medium was replenished at least every third day. Cells were pre-expanded for up to eight days before being cryo-preserved using CryoStor10 (CS10, STEMCELL Technologies). Upon thawing cells were validated for CD34 expression.

Priming of HSPC-pDCs

Priming of HSPC-pDCs was performed as previously described [10]. Isolated HSPC-pDCs were primed in the same medium as the differentiation was done in (either RPMI 1640, SFEM II or DC medium). Medium was depleted for growth factors and only supplemented P/S or IL-3 (20 ng/mL). pDCs were primed with 250 U/mL IFN-β (PBL Assay Science) and 250 U/mL IFN-γ (Peprotech) or left unprimed. For HSPC-pDCs differentiated in DC medium, medium was also supplemented with 50 μg/mL ascorbic acid. Cells were primed for three days before being phenotypically or functionally characterized.

TLR7 or TLR9 Agonist Activation

To analyze the capacity of HSPC-pDCs to produce type I IFN, 4×10⁴ pDCs were seeded out in 96-well plates in the same medium as the differentiation was done in (either RPMI 1640, SFEM II or DC medium). Medium was devoid of growth factors and only supplemented with P/S and IL-3 (20 ng/mL). Cells were subsequently stimulated with agonists directed against TLR7 (R837, tlrl-imq, InvivoGen) or TLR9 (CpG-A 2216, tlrl-2216-1, InvivoGen) at a final concentration of 2.5 μg/mL. Twenty hours post stimulation supernatants were harvested and cryopreserved at −20° C. until analysis.

Assessment of Functional Type I IFN

To quantify functional type I IFN, the reporter cell line HEK-blue IFN-α/β was utilized, according to the manufacturer's instructions (InvivoGen). The cell line was maintained in DMEM+Glutamax-1 (Gibco®, Life Technologies), supplemented with 10% heat-inactivated FCS, 100 μg/mL streptomycin and 200 U/mL penicillin (Gibco, Life Technologies), 100 μg/mL normocin (InvivoGen), 30 μg/mL blasticidin (InvivoGen) and 100 μg/mL zeocin (InvivoGen). Cells were passaged using 1× trypsin (Gibco, Life Technologies) and were not passaged more than 20 times. The HEK-blue IFN-α/β cell line has been generated by stable transfection of HEK293 cells to express IRF9 and STAT2. Moreover, the cell line has been modified to express a reporter gene encoding secreted alkaline phosphathase (SEAP) under the control of ISG54 promotor. Activity of SEAP was assessed using QUANTI-Blue (InvivoGen). Color change was measured at an optical density (OD) of 620 nm using the SpectraMax iD3 platereader (Molecular Devices). For the generation of standard curve, hIFNa2 (PBL Assay Science) was used, and ranged from 2 to 500 U/mL.

Phenotypic Analysis of Cells Using Flow Cytometry

Flow cytometry was used to immunophenotype pDCs. Briefly, cells were spun down and resuspended in 100 μL PBS. Cells were stained with Ghost Dye Red 780 Viability Dye (13-0865, Tonbo) for 30 min before being washed with FACS buffer (PBS with 2% FCS and 1 mM EDTA). Cells were subsequently resuspended in 50 μL FACS buffer and stained with the following antibodies: FITC anti-human Lineage Cocktail (CD3 (UCHT1), CD14 (HCD14), CD16 (3G8), CD19 (HIB19), CD20 (2H7) and CD56 (HCD56), 348801, BioLegend), APC anti-human CD11c (3.9, 20-0116-T100, TonboBio), PE anti-human CD123 (6H6, 12-1239-42, eBioscience), PE-Cy7 anti-human CD303 (201a, 25-9818-42, eBioscience). Cells were stained for 30 min before being washed three times in FACS buffer. To validate the expression CD34+ on HSPCs, cells were stained with PE-Cy7 anti-human CD34 (581, 343516, BioLegend) for 30 min, washed three times in FACS buffer before being resuspended in FACS buffer. PI was added to the cells as a viability dye in a concentration of 1:100 (13-6990 Tonbo Bioscience).

Fluorescence intensities were measured using the Quanteon flow cytometer equipped with 4 lasers (405 nm, 488 nm, 561 nm, and 637 nm) and 29 photomultipliers (PMT) detectors or the NovoCyte flow cytometer with two lasers (488 nm and 561 nm) and 12 PMT detectors (ACEA Biosciences, Inc.). Data analysis was done using FlowJo (Version 10, Tree Star, Ashland, OR, USA). Individual gating strategies are depicted in supplementary figures and outlined in figure legends.

Statistical Analysis

All data were plotted using Graphpad Prism 8.0 (GraphPad Software, San Diego, CA. USA). The data are shown as means of biological replicates+/−standard error of mean (+/−SEM). Statistically significant differences between groups were determined using One-way or Two-way ANOVO, followed by Bonferroni post hoc test. *P≤0.05, ** P≤0.01, *** P≤0.0001.

### Example 2—Low-Density HSPC Culture Improves Expansion and Yield of pDCs

Aim of Study

To determine the effect of low-density expansion of HSPCs on yield.

Materials and Methods

See example 1.

Results

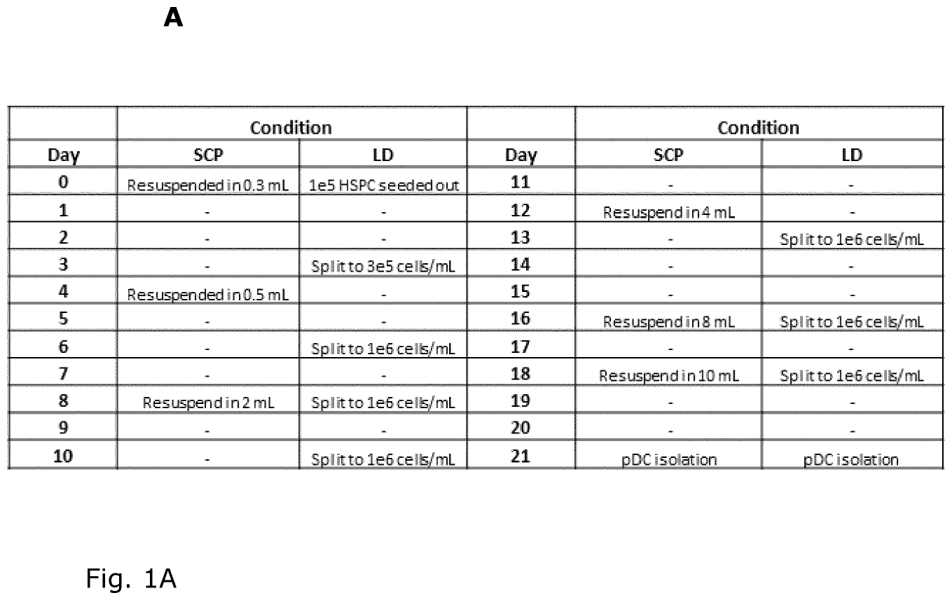

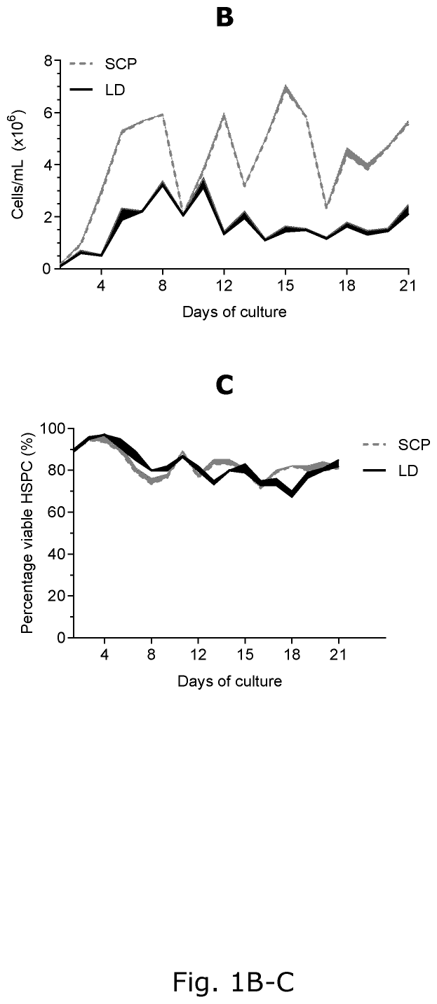

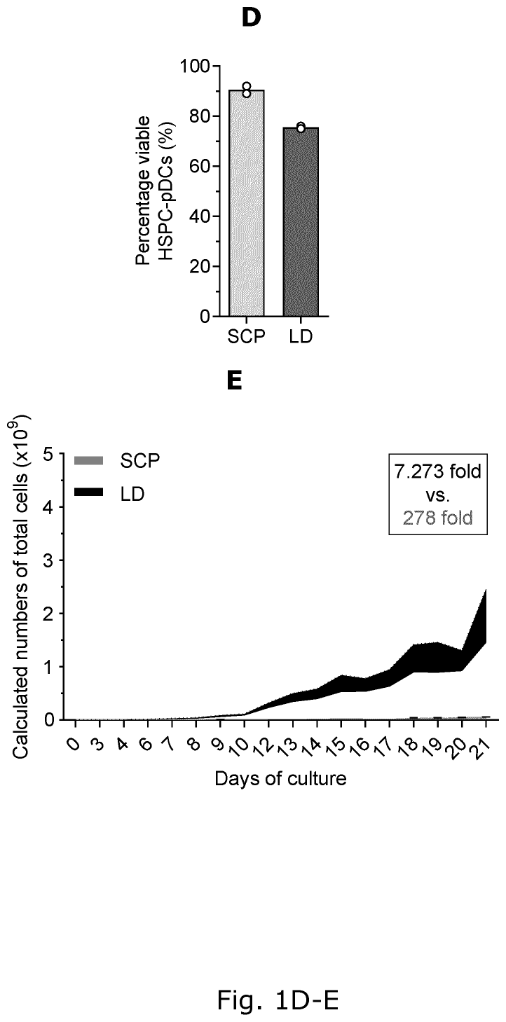

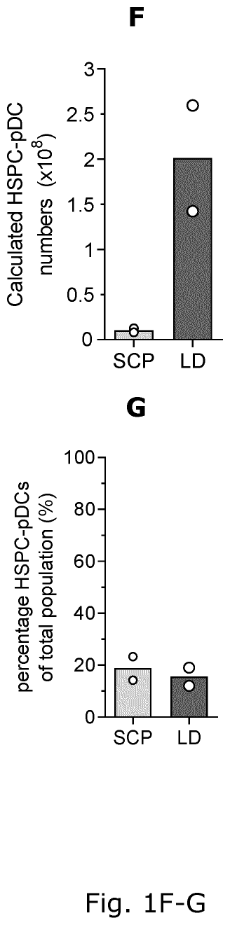

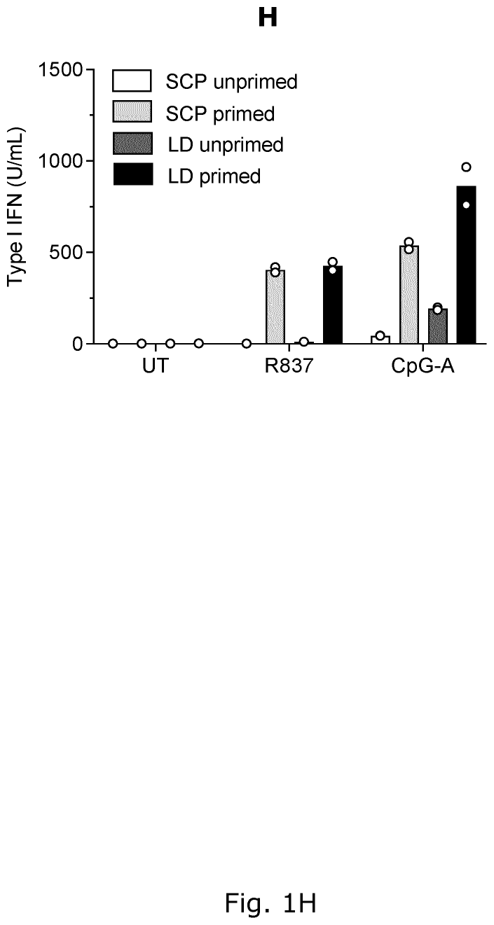

A previously published protocol for HSPC-to-pDC differentiation made use of a high-density culturing paradigm based on medium change on fixed days using fixed volumes of medium. Prior work has demonstrated that culturing HSPCs ex vivo at low-density culture conditions stimulates the transition of HSPCs into the cell cycle, thereby supporting the expansion of HSPCs [11]. Therefore, we compared our standard culture protocol (SCP) to a low-density (LD) protocol where cells were split more frequently and not allowed to reach a density exceeding 5×10⁶ cells/mL at any time point (see FIG. 1A-D) for culturing overview). During the 21-day culture, we observed that the low-density protocol led to a remarkably higher expansion of HSPCs compared to the standard protocol (FIG. 1E). Starting from 2×10⁵ HSPCs, we were able to obtain an average number of 1.5×10⁹ (+0.7×10⁹) total cells compared to 0.055×10⁹ (+0.001×10⁹) in the standard condition (FIG. 1E).

Next, we isolated HSPC-pDCs using immunomagnetic depletion of non-pDCs. Importantly, we found that the low-density culture protocol also highly increased HSPC-pDC numbers. Starting from 2×10⁵ HSPCs, the average yield of isolated HSPC-pDCs was 201×10⁶ (±58.7×10⁶) versus 10.4×10⁶ (±2.3×10⁶) HSPC-pDCs in the standard condition (FIG. 1F). This is an average yield of 1005 (±293) HSPC-pDCs per single CD34⁺ HSPC, which is an average improvement of 19-fold over the standard condition. The culture density did not affect the viability of the cells during the 21-day culture period (FIG. 1C), nor did it influence the fraction of HSPC-pDCs among the total cells that were generated at day 21 (FIG. 1G). However, a small decrease in viability of the isolated HSPC-pDCs from the low-density culture was detected (FIG. 1D). pDCs are known to secrete very large amounts of type I IFNs in response to agonists directed against TLR7 or TLR9 [1]. Previously, we have shown that isolated HSPC-pDCs require a priming by adding type I and II IFNs to the culture medium to become functionally active and responsive to TLR7 and TLR9 agonists [10]. We therefore next primed the isolated HSPC-pDCs for three days, and subsequently stimulated the cells with TLR7 and TLR9 agonists. Evaluation of type I IFN responses of HSPC-pDCs generated under low-density conditions versus standard conditions showed no difference when stimulated with a TLR7 agonist and a minor improvement for the low-density condition when stimulated with a TLR9 agonist (FIG. 1H). Phenotypic analysis of pDC surface markers by flow cytometry of the isolated HSPC-pDCs (Lin⁻CD11c⁻CD123⁺CD303⁺) showed no obvious difference between the two culture conditions (data not shown).

Conclusion

The present example shows that low density expansion of HSPCs increases yield of HSPC-pDCs.

### Example 3—Serum-Free Medium Improves Expansion of HSPCs and Increases pDC Yield

Aim of Study

Traditional cell culture medium remains a challenge for therapeutic use owing to the ill-defined and highly variable nature of serum. As we have previously shown that HSPC-pDCs could be generated using serum-free medium, we rationalized that combining commercially available serum-free medium (SFEM II medium) with our newly defined low-density condition (see example 2), would constitute a more streamlined HSPC-pDC production protocol with possible improvements in functionality and yield [10].

Additionally, we wanted to evaluate if the higher expansion rate of HSPCs at low-density culture would enable earlier isolation of functional HSPC-pDCs, thereby reducing manufacturing cost and duration of a potential future clinical product.

Materials and Methods

The high yield obtained with the low-density protocol (example 2) prompted reducing the starting cell numbers from 2×10⁵ to 1×10⁵ HSPCs and still generate sufficient numbers of HSPC-pDCs for subsequent analysis.

See also example 1 and 2.

Results

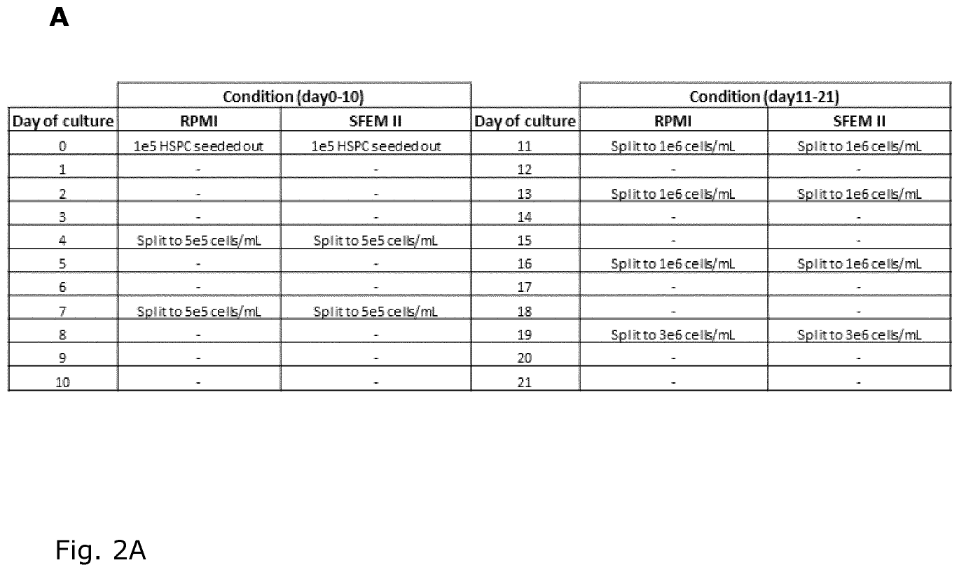

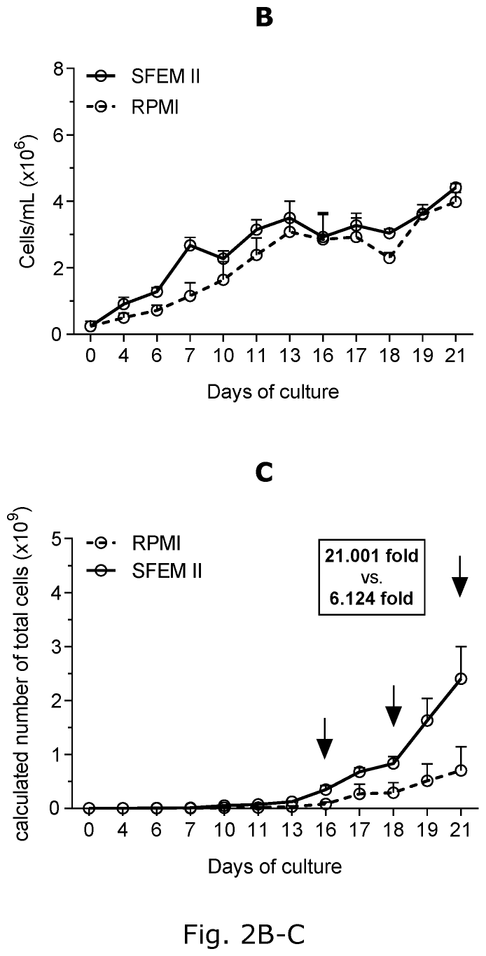

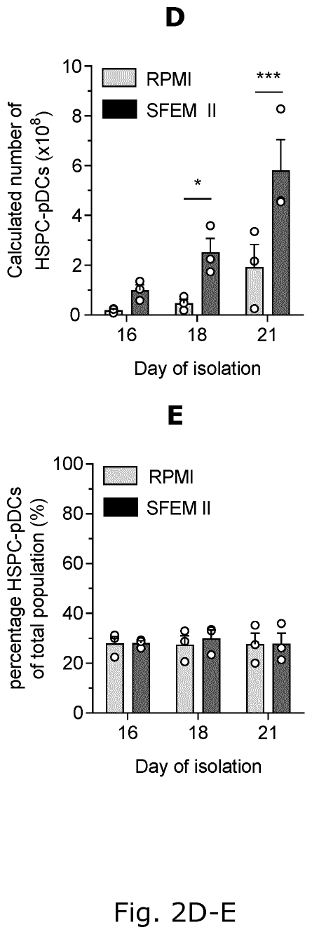

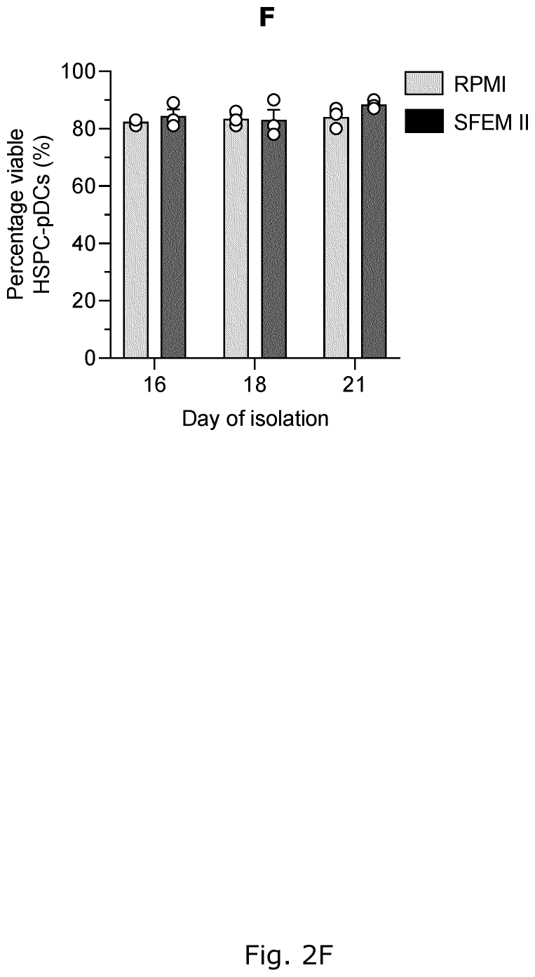

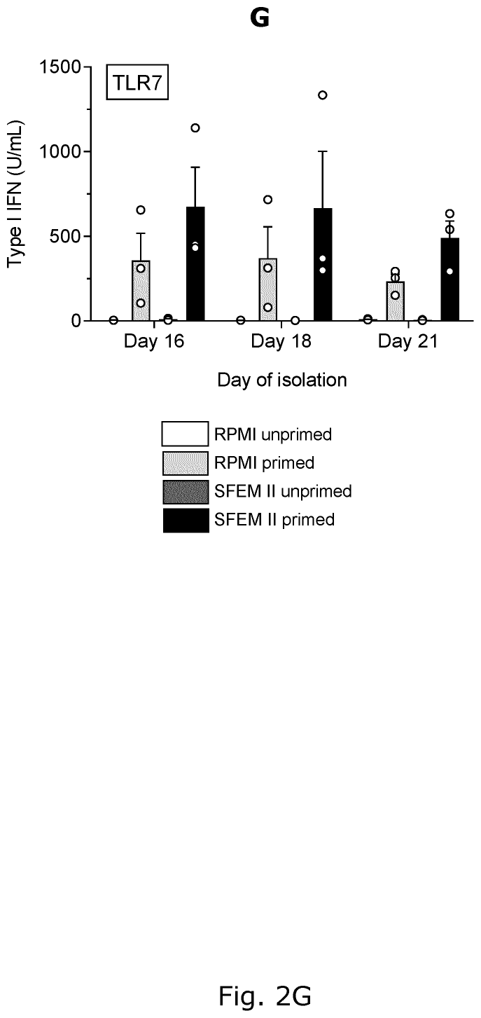

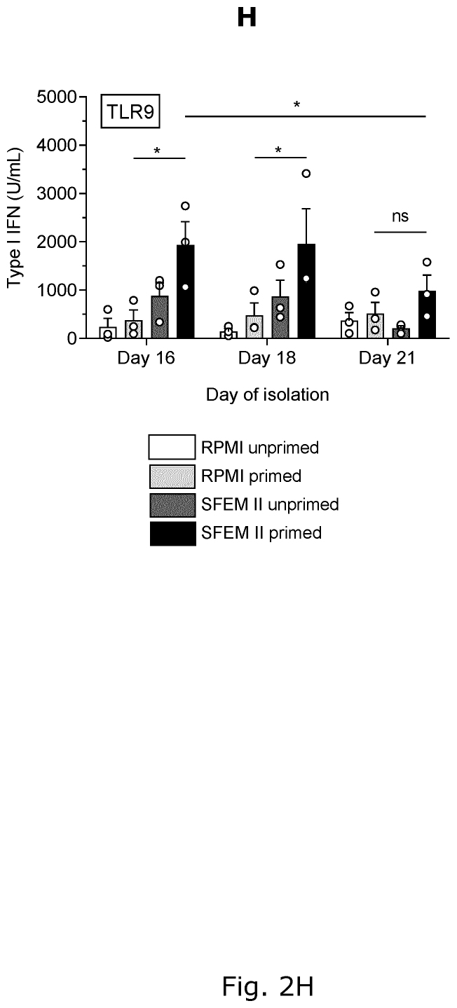

We then compared SFEM II to the conventional serum-based culture medium (RPMI) at low-density conditions (See FIG. 2A-B for culture overview). Cells were cultured for 21 days while HSPC-pDCs were isolated by immunomagnetic selection at days 16, 18, and 21. We found that SFEM II significantly promoted the proliferation of HSPCs compared to RPMI with serum (FIG. 2C). Starting from 1×10⁵ HSPCs, an average of 2.4×10⁹ (+0.6×10⁹) total cells were generated at Day 21 with SFEM II versus 0.7×10⁹ (+0.4×10⁹) total cells for RPMI (FIG. 2C). The high expansion rate using SFEM II also translated to a significantly higher yield of isolated HSPC-pDCs at all three time points of isolation, with a maximum of 580×10⁶ HSPC-pDCs (+123×10⁶) HSPC-pDCs isolated after 21 days of culture using SFEM II (FIG. 2D). Even after 16 days of differentiation, 100×10⁶ (+22.2×10⁶) HSPC-pDCs could be isolated starting from only 0.1×10⁶ HSPC. The transition to SFEM II did not affect the proportion of HSPC-pDCs in the cell culture, as similar percentages of HSPC-pDCs were observed across all three isolation time points, indicating that pDCs differentiate continuously during the culture (FIG. 2E). Importantly, no difference in viability of the isolated HSPC-pDCs was observed at any time point or condition (FIG. 2F). We next performed TLR7 and TLR9 activation assays of the produced HSPC-pDCs, which showed that cells generated using SFEM II displayed a higher capacity to produce type I IFN (FIG. 2G-H). Interestingly, we observed that HSPC-pDCs that had been isolated at earlier time-points produced significantly more type I IFN upon TLR9 activation, suggesting that there is a balance between proliferation potential and the immunotherapeutic properties of HSPC-pDCs. Phenotypic analysis of the pDC-related surface markers CD123 and CD303 on lin⁻CD11c⁻ cells showed no differences across time points and conditions (FIG. 2I-J).

Conclusion

Taken together, these data show that HSPC expansion and functionality of isolated HSPC-pDC generation were improved using the serum-free medium SFEM II compared to RPMI supplemented with serum. This enables earlier isolation of HSPC-pDCs while improving functionality of the cells and preserving high cell yield.

### Example 4—Low-Density Culture Supplemented with the Pyrimido-Indole Derivative UM171 Allows HSPC Pre-Expansion

Aim of Study

In the pursuit of increasing HSPC-pDC yield further, we next investigated HSPC pre-expansion before initiating the pDC differentiation protocol. A fundamental limitation to HSPC ex vivo culturing is the rapid differentiation of stem and progenitor cells, which in turn produces inhibitory feedback signals that limits stem cell self-renewal. Recent publications have found that ex vivo culturing of HSPCs at low densities combined with the small molecules UM171 and SR1 promote self-renewal of primitive hematopoietic progenitor cells and long-term repopulating hematopoietic stem cells (LT-HSCs) [11, 12]. Based on this, we set out to test if a pre-expansion HSPC protocol could be implemented prior to pDC differentiation, potentially allowing very limited numbers of CD34+HSPCs to produce high yields of HSPC-pDCs.

Materials and Methods

See examples 1-3.

Results

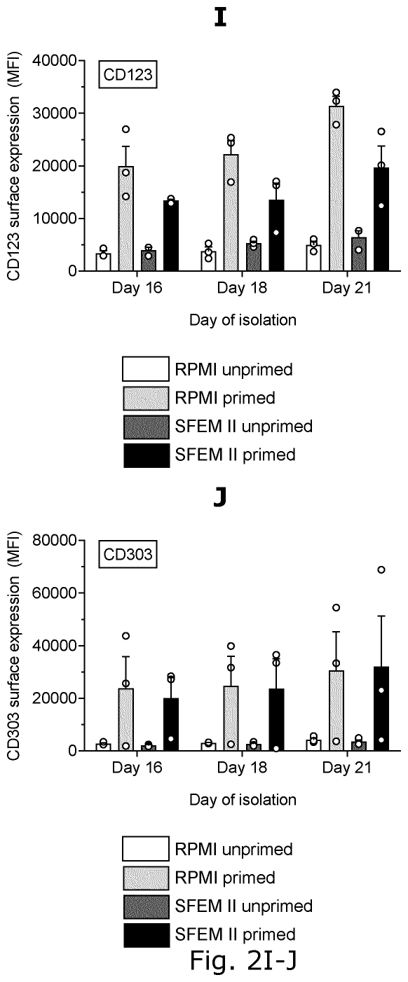

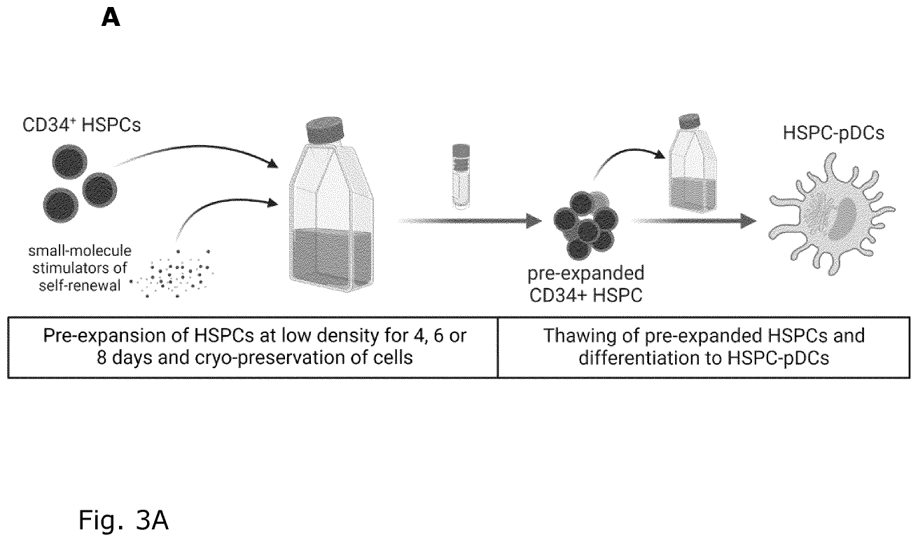

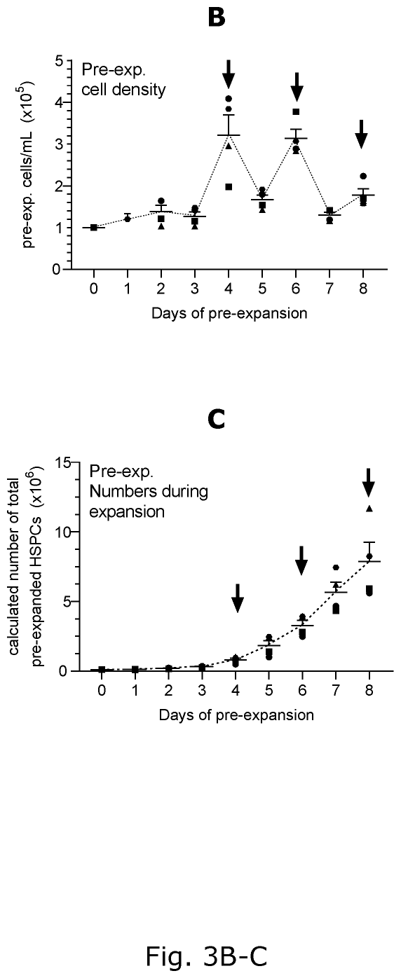

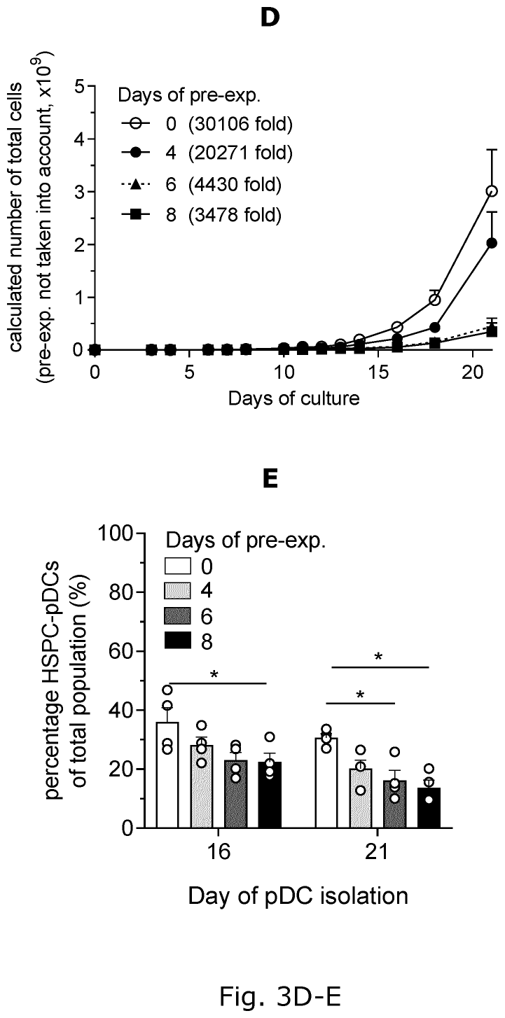

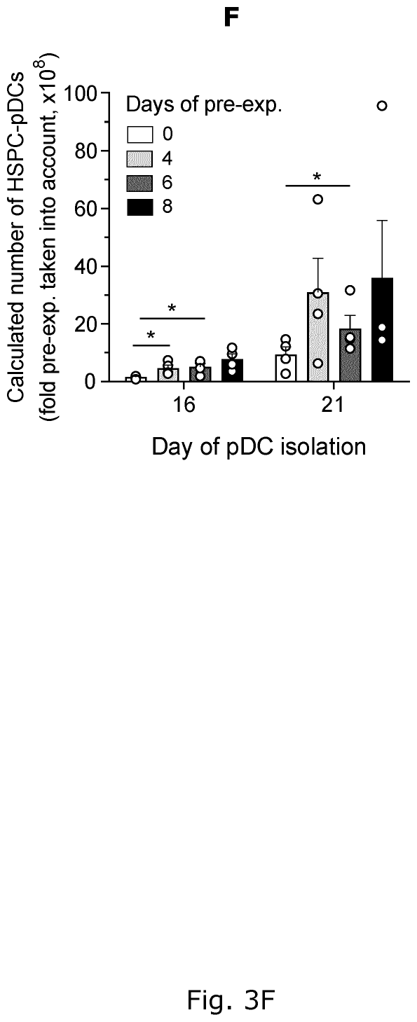

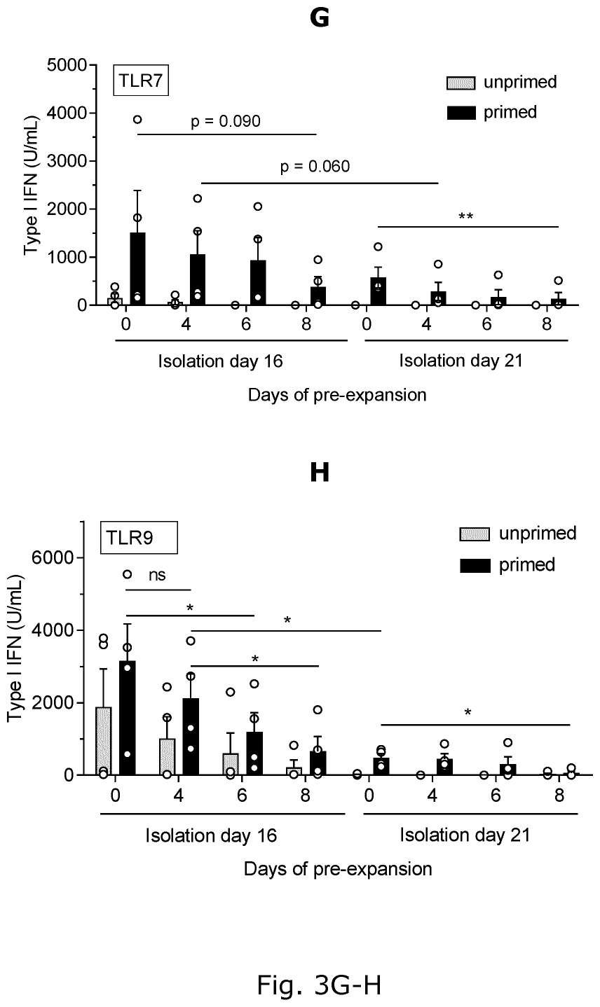

HSPCs were cultured at low-density concentrations (0.1-0.5×10⁶ cells/mL) for up to 8 days (FIG. 3A), during which the cells expanded significantly with an average of 78 (±14) fold expansion for the 8-day pre-expansion (FIG. 3B-C). Expanded HSPCs were cryo-preserved after 4, 6, or 8 days of expansion to enable initiation of parallel pDC differentiation studies. Upon thawing, expanded HSPCs remained viable and positive (>95%) for the HSPC surface marker CD34⁺ with no difference compared to HSPCs that had not been expanded (Data not shown). However, surface expression levels (MFI) of CD34 appeared to increase during the first four initial days of expansion, and then decrease again over time (data not shown). Next, using the established low-density SFEM II culture protocol (see example 2), parallel pDC differentiations were initiated with equal numbers of HSPC-pDCs isolated after the different pre-expansion durations. We observed that HSPCs that had undergone pre-expansion for 6 and 8 days showed decreased expansion during pDC differentiation, but viabilities throughout differentiation remained high for all conditions (FIG. 3D and data not shown). The stagnated growth correlated with increased numbers of adherent cells during later days of pDC differentiation, and these adherent cells displayed a morphology with long protrusions indicative of cDCs or macrophages (data not shown). This indicates that prolonged pre-expansion affects differentiation and the proliferative capacity of hematopoietic progenitors, which in turn may influence pDC differentiation. Accordingly, when initiating HSPC-pDC differentiation from the same starting cell numbers, lower yields of immunomagnetically separated HSPC-pDCs were observed upon prolonged pre-expansion, in particular when combined with 21 days of pDC differentiation (data not shown). A progressive decline in the percentage of HSPC-pDCs of the total cell population was also observed as the cells had been pre-expanded for longer durations (FIG. 3E). Nevertheless, when fold pre-expansion was taken into account, very high numbers of pDCs could be generated with pre-expansion (FIG. 3F). With a four day pre-expansion of HSPCs and a 21-day pDCs differentiation, 3.1×10⁹ (±1.2×10⁹) HSPC-pDCs could be generated versus 0.9×10⁹ (±0.3×10⁹) HSPC-pDCs when no pre-expansion was applied. Similarly, early pDC isolation at day 16 yielded an average of 4.7×10⁸ (±1.1×10⁸) HSPC-pDCs when a 4-day pre-expansion was applied (FIG. 3F).

Next, we set up experiments where we primed the generated HSPC-pDCs and evaluated phenotypic and functional parameters of the cells. Interestingly, we found that the functional capacity of HSPC-pDCs to secrete type I IFN upon activation with TLR7 or TLR9 agonists was highly affected when cells were pre-expanded for a prolonged time (FIG. 3G-H). This was in particular evident for the TLR9-mediated type I IFN response, which was significantly decreased when applying 6 or 8 days of pre-expansion. Conversely, surface expression of CD123 and CD303 was not affected by culture duration (Data not shown).

Conclusion

Overall, these data show a trend of reduced functionality with longer time in culture, which might reflect a type of functional exhaustion during prolonged cell culture. Collectively, we here show that prolonged pre-expansion, as well as extended pDC differentiation culture drastically affect the functionality of generated HSPC-pDCs. Nevertheless, when a limited period of pre-expansion of 4 days is combined with an early isolation of HSPC-pDCs of 16 days, high yields of functional HSPC-pDCs can be generated.

### Example 5—the Use of a CGMP-Compliant Medium Abolishes the Ability of HSPC-pDCs to Respond to TLR Agonists

Aim of Study

Recent technical advances in the use of defined media and synthetically made culture substrates have significantly improved both the simplicity and predictability of growing and differentiating stem cells. As clinical data highlight the promise of pDCs in immunotherapy, and since clinical cell products must be produced under CGMP, we pursued to implement a CGMP-compliant medium to our culture protocol.

Materials and Method

See examples 1-4.

Results

We performed pDC differentiation experiments using two commercially available CGMP-compliant serum-free media ‘SCGM’ and ‘DC Medium’. As we had already used a commercially available serum-free medium (SFEM II), we assumed an uncomplicated transition.

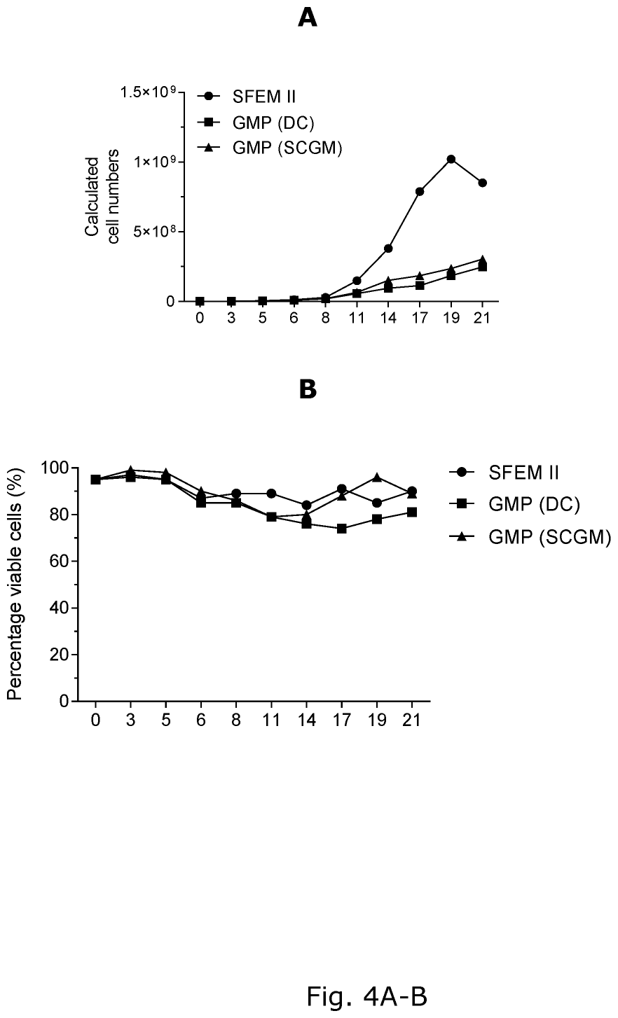

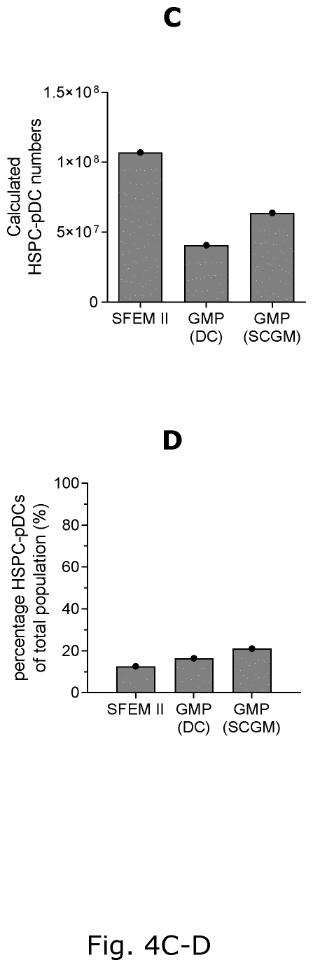

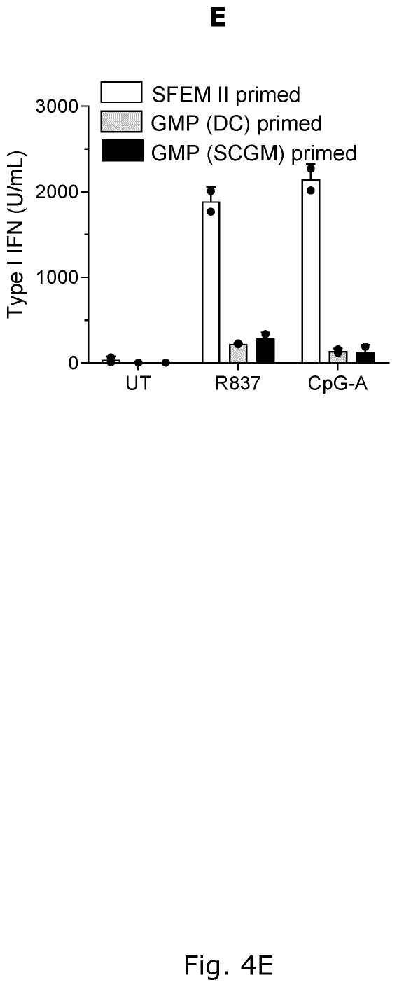

Interestingly, we found that expansion of cells during pDC differentiation was highly reduced when HSPCs were cultured in CGMP medium (FIG. 4A). However, cell viabilities remained high throughout the culture period, but the number of HSPC-pDCs that could be isolated from the culture was reduced in accordance with the lower expansion (FIG. 4B-C). Viabilities of isolated HSPC-pDCs and their proportion of the total cell population were largely unaffected (FIG. 4D and data not shown) lending us to believe that their functionality would remain unaffected.

Remarkably, while isolated HSPC-pDCs from all three culture conditions phenotypically expressed the pDC markers CD123 and CD303 to the same extent, cells cultured in the two CGMP-compliant media were found to either completely lack or have drastically reduced capacity to produce type I IFN upon TLR7 or TLR9 agonist activation (FIG. 4E).

Conclusion

These data shows that it is not a simple task to switch to a CGMP medium, since the capacity to produce type I IFN upon TLR7 or TLR9 agonist activation were drastically reduced.

### Example 6—Ascorbic Acid Rescues the Function of HSPC-pDCs Produced Using CGMP Media

Aim of Study

Vitamin C (Ascorbic acid, AA) is an essential vitamin in humans known to have pleiotropic functions in cellular biology, including immune cell function and hematopoiesis. Interestingly, AA has been shown to be involved in type I IFN immune responses. Unlike humans, mice can synthesize AA but interestingly, transgenic mice lacking the capacity to synthesize AA show diminished capacity to produce type I IFN upon TLR activation and influenza infection, indicating that AA plays a role in either TLR activity or pDC development. While no reports provide hard evidence of a role in pDC development of function, one study has shown that AA supplementation can increase DC yield during ex vivo differentiation from HSPC, but its significance in pDC functionality was not investigated [9]. Given the evidence for the role of AA in hematopoiesis and IFN responses, we hypothesized that AA addition to our culture medium would improve expansion and viability of HSPCs during pDC differentiation. To that end, we explored a culture system focusing on the CGMP-compliant ‘DC Medium’ where we investigated the addition of physiological concentration of AA (50 μM) during HSPC-pDC differentiation [13].

Materials and Methods

See example 1-5.

Results

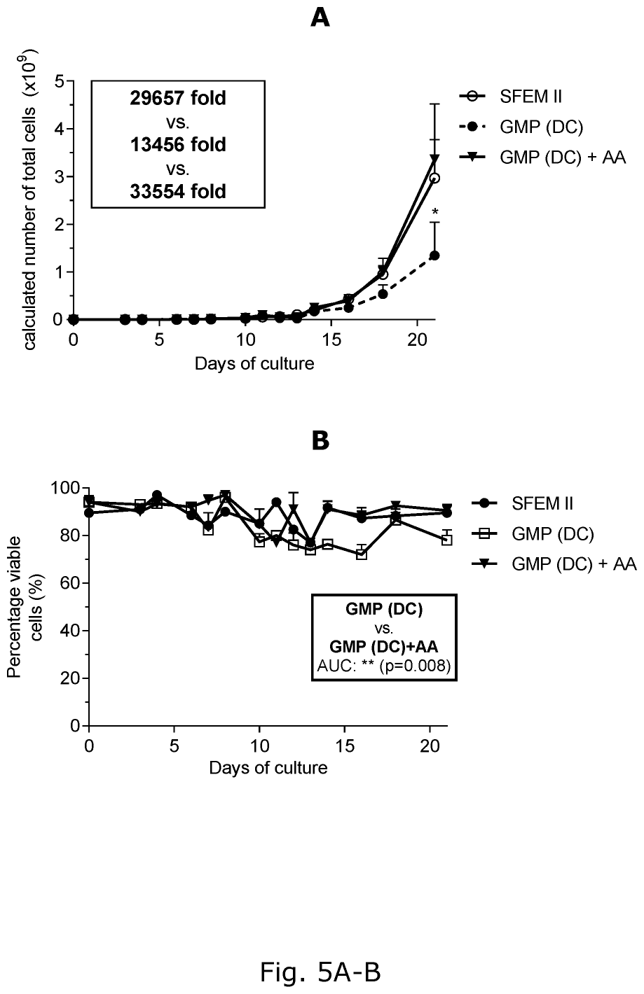

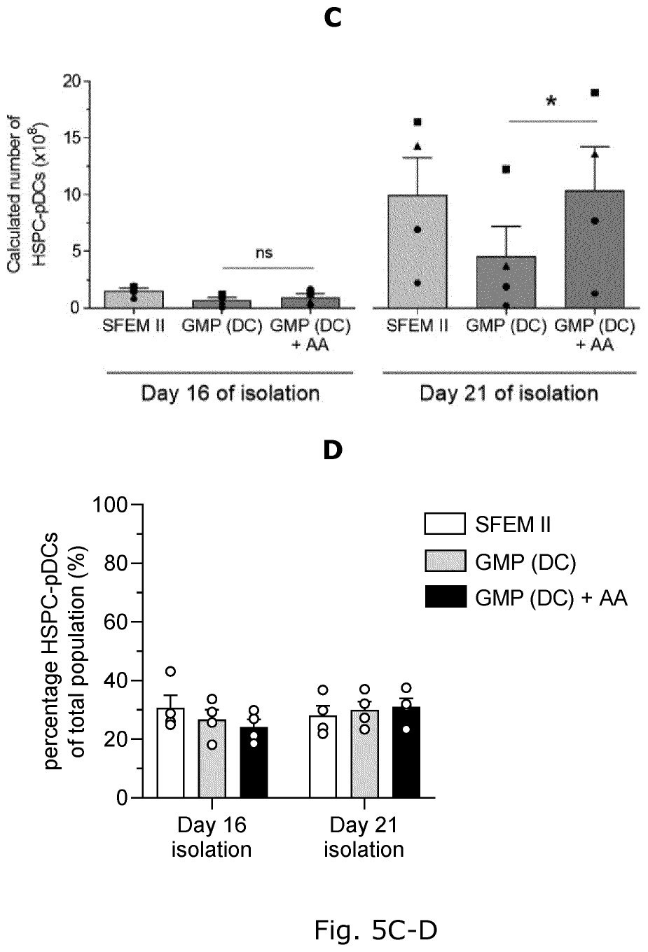

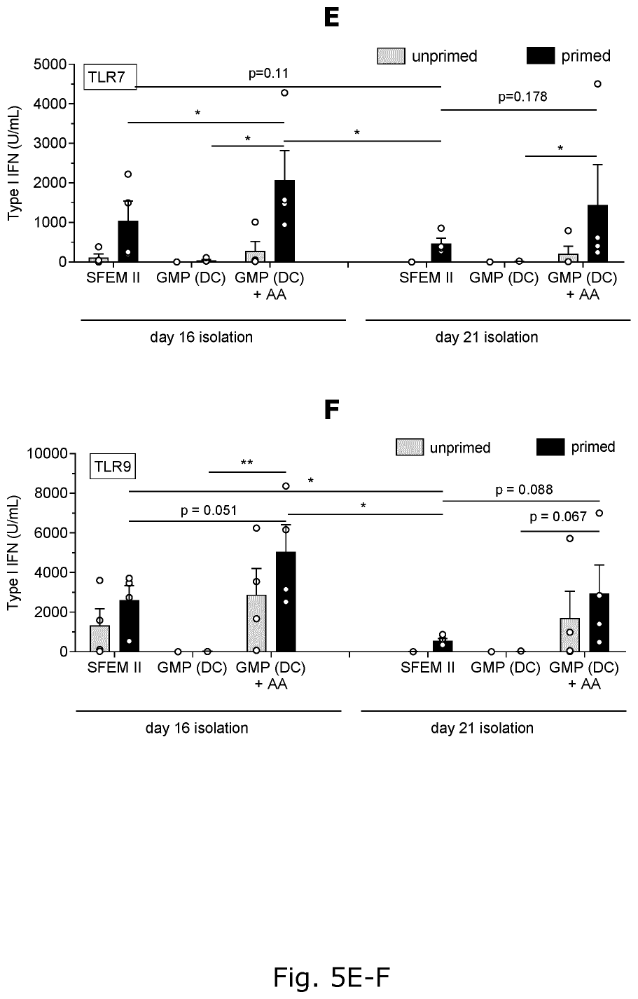

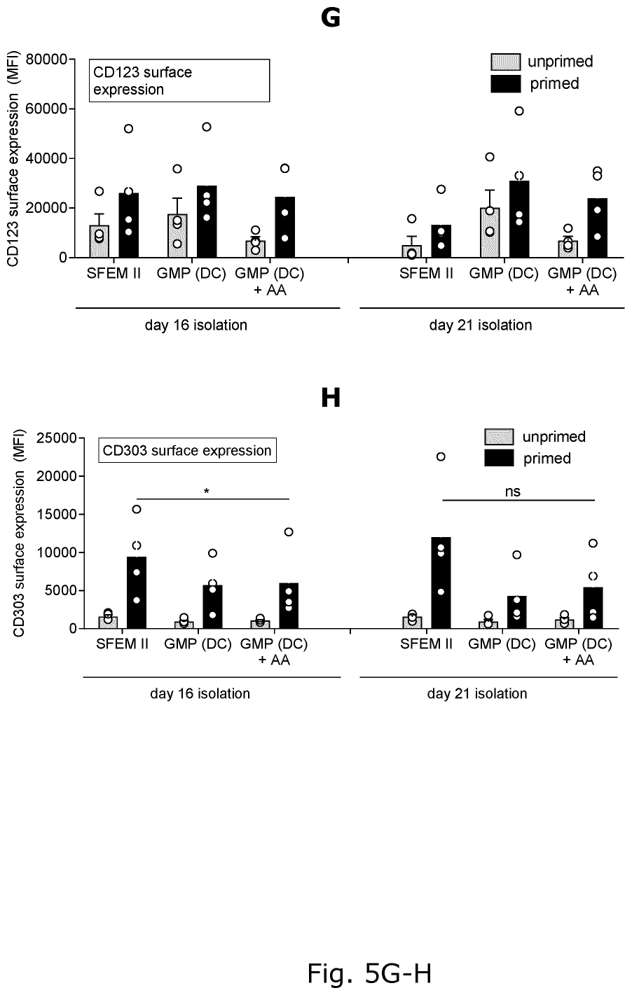

We found that DC medium supplemented with AA significantly promoted expansion during HSPC-pDC differentiation up to similar level as the SFEM II culture condition (expansion of 33,554 fold (±11,664) for DC medium+AA versus 13,456 fold (±6,953) for DC medium alone) (FIG. 5A). Moreover, addition of AA significantly improved viability of expanding HSPCs during culture, in particular during later stages of culture (Day 12+) (FIG. 5B). Additionally, we also found the viability of HSPC-pDCs isolated at day 21 to be significantly increased upon AA addition compared to DC medium alone (92.0%±3.4% versus 72.8%±3.2%) (data not shown). Accordingly, the yield of HSPC-pDCs was significantly increased upon AA supplementation for HSPC-pDCs isolated at day 21 (1.0×10⁹+0.4×10⁹ HSPC-pDCs for DC medium+AA versus 0.5×10⁹+0.3×10⁹ for DC medium alone per 0.1×10⁶ CD34⁺ HSPCs) (FIG. 5C), without affecting the percentage of HSPC-pDCs of the total population of cells (FIG. 5D). No statistical significant differences were observed in the yield of HSPC-pDCs isolated at day 16 of culture for the different conditions. Next, we stimulated primed and unprimed HSPC-pDCs with synthetic TLR7 and TLR9 agonists and evaluated the type I IFN response. Interestingly, we found that HSPC-pDCs generated from the AA culture consistently elicited a robust type I IFN response in contrast to DC medium alone for cells isolated both on day 16 and 21 (FIG. 5E-F). The response was even found to exceed the response of HSPC-pDCs generated using SFEM II medium. As previously observed, HSPC-pDCs isolated at 16 days of culture displayed improved capacity to produce type I IFN upon TLR7 or TLR9 activation compared to cells isolated at day 21 (FIG. 5E-F). Upon analysis of surface expression of the markers CD123 and CD303, we found that pDCs isolated from AA-supplemented culture at day 16 displayed lower expression levels of CD303. However, no statistical significance was observed in the percentage of cells expressing CD303 between the conditions (FIG. 5G-H). Notably, a small distinct population of HSPC-pDCs showing high surface expression of CD123 was observed upon AA supplementation (data not shown). This population was also observed for SFEM II culture conditions, albeit not to the same extent and not for all donors analyzed.

Conclusion

Together, these data demonstrate that AA is essential for ex vivo differentiation of pDCs from HSPCs when using CGMP-compliant medium, by both increasing expansion of HSPCs, improving viability of expanding cells and isolated HSPC-pDCs, as well functionality of isolated HSPC-pDCs.

### Example 7—Generation of HSPC-pDCs from Whole Blood

Aim of Study

To this date, two clinical trials have reported the use of autologous peripheral blood-derived pDCs as a cell-based cancer immunotherapy [5, 6]. In both trials, pDCs were found to induce favorable anti-tumoral responses by effectively promoting anti-tumoral responses while being well-tolerated. Despite the use of leukapheresis, the low numbers of peripheral blood pDCs that can be isolated remains a key limiting factor. In contrast, high numbers of pDCs can be generated by differentiation of patient-derived HSPCs isolated either by direct bone marrow aspiration or by blood leukapheresis following administration of mobilizing regiments. However, these procedures are invasive, painful, associated with side effects, or require multi-day doses of mobilizing drugs. For research purposes, e.g. to investigate HSPC-pDC functions and antigen presentation to autologous memory cells, HSPCs acquired by these procedures are costly and can be a challenge to procure. An alternative source for HSPCs is peripheral whole blood where a limited number of naturally circulating CD34+HSPCs (cHSPCs) can be found. However, the rarity of these cells have so far limited their use for therapeutic purposes and furthermore, their capacity for self-renewal and differentiation capacity has also been reported to be much lower than other sources of HSPCs. Based on our data, we reasoned that our high-yield differentiation protocol would allow therapeutically relevant numbers of HSPC-pDCs to be generated from cHSPCs.

Materials and Methods

See example 1.

Results

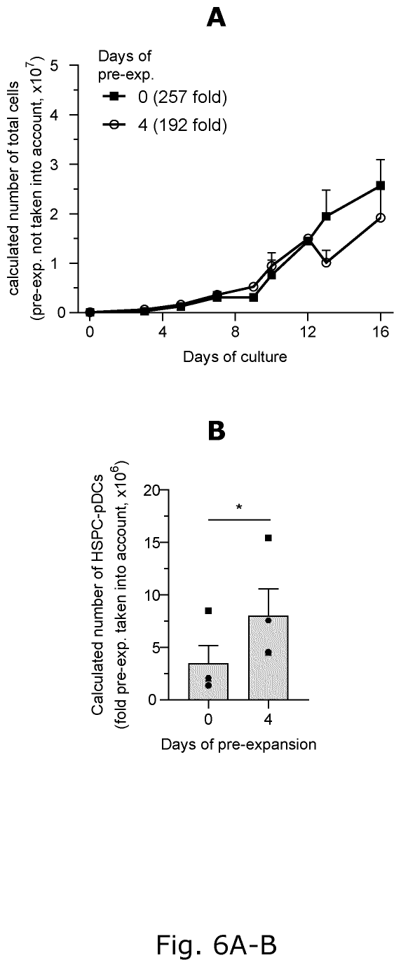

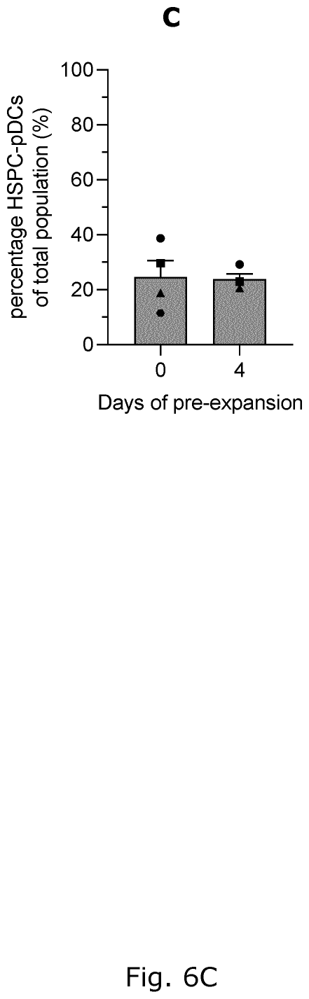

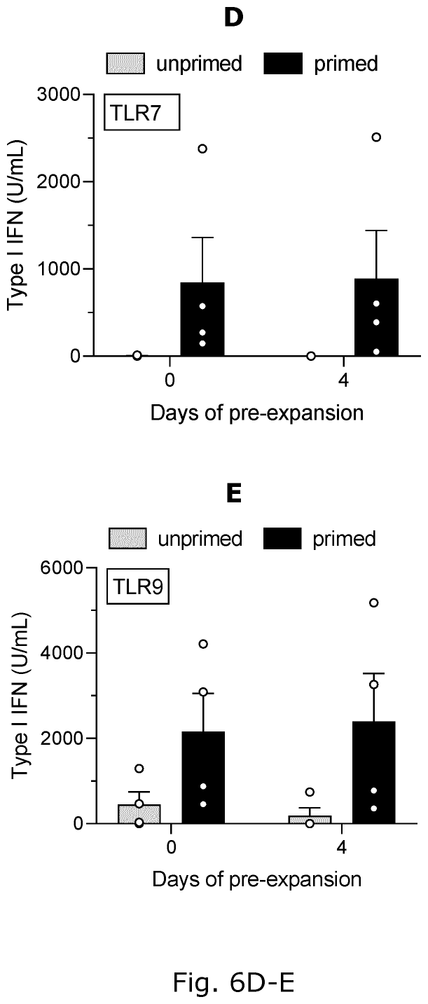

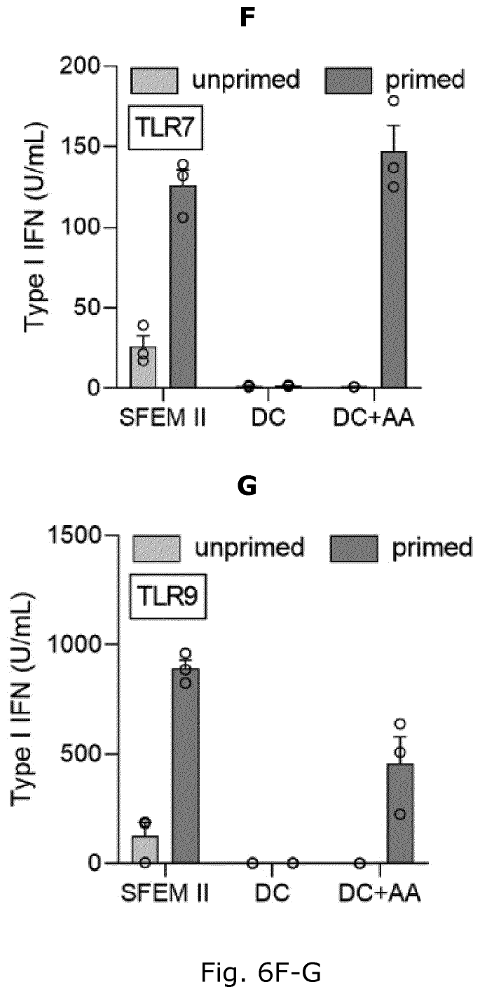

We obtained buffy coats (from around 450 mL of whole blood) and isolated CD34⁺ cHSPCs using CD34 immunomagnetic positive selection. We obtained an average number of 1.1×10⁶ CD34⁺ cHSPCs (±0.6×10⁶ cHSPCs), corresponding to 2457 cHSPCs (±1307 cHSPCs) per mL of blood in line with previous observations (data not shown). Next, we systematically evaluated if a pre-expansion of cHSPC and subsequent pDC generation would be feasible. We therefore initiated parallel 16-day pDC differentiation cultures of cryopreserved cHSPCs from the same donors that had either not been pre-expanded or pre-expanded for 4 days and subsequently cryo-preserved. For a fully CGMP-compliant protocol, pre-expansion was performed using CGMP-compliant SCGM medium and pDC differentiation was performed using CGMP-compliant DC medium+AA. During pre-expansion, cHSPCs expanded 4.6 fold (±1.5 fold) and retained CD34 expression (data not shown). During the 16 days of pDC differentiation, we observed a 257-fold (±91 fold) expansion of total cells for non pre-expanded cHSPC versus 192-fold (±126 fold) for cHSPC pre-expanded for 4 days (FIG. 6A). When starting from 1×10⁵ cHSPCs, the total yield of cHSPC-pDCs following immunomagnetic selection was 3.5×10⁶ cHSPC-pDCs (±2.9×10⁶) without pre-expansion (FIG. 6B). Using an optimized setup with 4 days of pre-expansion, an average yield of 8.0×10⁶ cHSPC-pDCs (±4.5×10⁶) was observed (FIG. 6B). For the pre-expanded condition, this corresponds to 80 cHSPC-pDCs generated per single cHSPC. Of the total cell population generated, cHSPC-pDCs accounted for an average of 23% for pre-expanded cHSPC (FIG. 6C). As expected, the observed pre-expansion, differentiation potential, and yield was less for cHSPCs compared to CB-HSPCs, whereas the frequency of pDCs obtained was similar. Importantly, cHSPC-pDCs were capable of producing type I IFN upon TLR7 or TLR9 stimulation, and the cells displayed a pDC surface phenotype similar to HSPC-pDCs derived from CB (FIG. 6D-E, and data not shown). As we had previously observed, AA was required for cHSPC-pDC TLR7 and TLR9-mediated type I IFN production (FIG. 6F-G).

Conclusion

Collectively, we demonstrate that high numbers of functional HSPC-pDCs can be generated from a simple blood sample, which highly simplifies the procedure for generating pDCs for basic studies of pDC biology and for immunotherapeutic purposes.

### Example 8—RNA-Sequencing of HSPC-pDCs

Aim of Study

To determine differences in gene expression profiles between HSPC-pDCs generated in the presence or absence of Ascorbic acid (AA).

Material and Methods

HSPCs were thawed and 1×10⁵ cells were seeded in DC medium with or without supplementation of ascorbic acid (AA). Following 16 days of differentiation with or without AA in the medium, pDCs were isolated by immunomagnetic depletion of non-pDCs, and HSPCs-pDCs were then primed for 72 hours with IFN-β/γ.

Following priming, HSPC-pDCs were stored in RNAprotect Cell Reagent (Qiagen) at −80° C. until total RNA was extracted using the RNeasy Plus Micro Kit (Qiagen), which efficiently eliminates genomic DNA without the need for DNase treatment. The total RNA was send to BGI Europe for RNA-seq. Here, a non-stranded & polyA-selected mRNA library was prepared from the total RNA and subjected to PE100 sequencing using the BGISEQ platform. The samples generated on average about 4.84 Gb bases per sample. Low quality reads were filtered and the remaining reads were mapped to the genome with an average mapping ratio with the reference genome at 92.74%, the average gene mapping ratio at 79.90%. In total, 18,412 genes were identified. Gene expression was calculated based on the reads, differentially expressed genes and gene ontology analyses were analyzed on BGI's software analysis platform Dr. Tom.

See also example 1.

Results

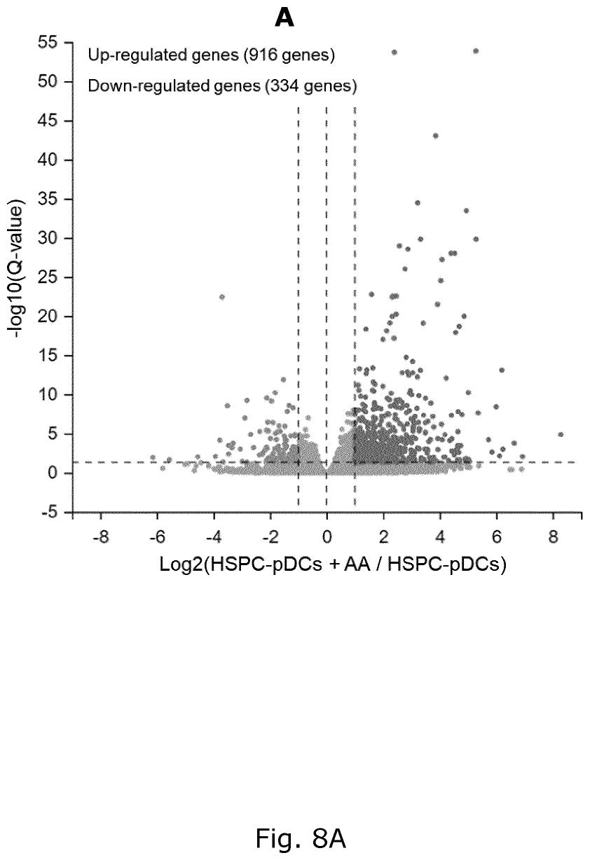

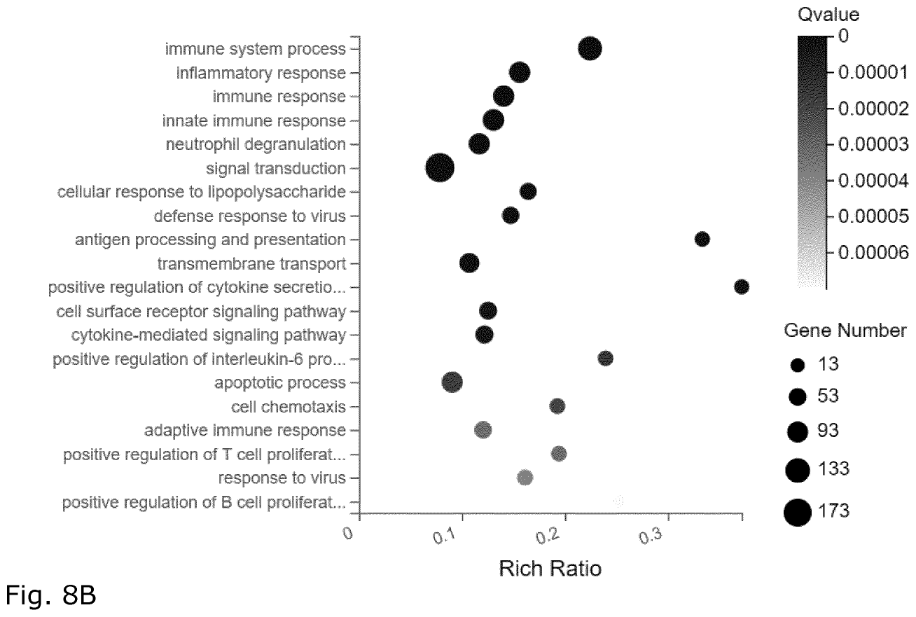

We obtained 916 upregulated genes and 334 downregulated genes in the condition with AA when applying the following threshold for up- and downregulated genes: |log 2FC|>=1 (FC=fold-change) and Qvalue <=0.05 (FIG. 8A).

The 20 most enriched biological processes for the differentially expressed genes in HSPC-pDCs generated with ascorbic acid were mainly related to immunological pathways (Figure (8B).

A list of the 50 most highly expressed genes in HSPC-pDCs generated with AA was generated. The inclusion criteria in this list was additionally that the observed upregulation was statistically significantly. These genes are listed in Table 1.

TABLE 1
Top 50 genes that were most highly expressed in HSPC-pDCs generated
with ascorbic acid (+AA) and also statistically significantly
upregulated compared to HSPC-pDCs generated without AA (−AA).
Shown are the official gene symbols, the log2 values of the fold-
change in gene expression levels upon addition of ascorbic acid
to the medium (log2[+AA/−AA]), the statistical significance
(Q-value) of the observation of differential expression, and the
average relative gene expression levels measured as Fragments
Per Kilobase Million (FPKM) in both conditions (−AA and +AA).

| | Log2 | Qvalue | −AA | +AA |
|---|---|---|---|---|
| Gene | (+AA/−AA) | (+AA/−AA) | FPKM | FPKM |
| ‘CD74’ | 0.841 | 3.24E−08 | 5008 | 8923 |
| ‘FTH1’ | 1.289 | 0.00845252 | 1395 | 3417 |
| ‘HLA- | 1.122 | 1.76E−04 | 1106 | 2401 |
| DRB1’ | | | | |
| ‘HLA-DPA1’ | 0.763 | 6.34E−04 | 1038 | 1750 |
| ‘IFITM3’ | 1.496 | 6.61E−06 | 494 | 1383 |
| ‘FCER1G’ | 0.509 | 0.033270138 | 961 | 1367 |
| ‘S100A11’ | 0.525 | 0.024890893 | 732 | 1054 |
| ‘DEFA1’ | 5.728 | 6.38E−05 | 20 | 1047 |
| ‘PSAP’ | 0.802 | 7.31E−04 | 600 | 1044 |
| ‘DEFA4’ | 4.651 | 1.65E−08 | 39 | 975 |
| ‘CTSD’ | 1.460 | 1.73E−06 | 323 | 893 |
| ‘GRN’ | 0.695 | 0.015829898 | 432 | 696 |
| ‘ITGB2’ | 0.599 | 9.28E−04 | 433 | 654 |
| ‘CD68’ | 1.365 | 3.20E−09 | 240 | 619 |
| ‘DEFA3’ | 4.431 | 0.001704486 | 27 | 593 |
| ‘TYMP’ | 0.679 | 0.016368204 | 348 | 553 |
| ‘CHI3L1’ | 2.178 | 0.024834793 | 108 | 496 |
| ‘SERPING1’ | 1.387 | 2.30E−05 | 181 | 471 |
| ‘CTSZ’ | 1.002 | 3.93E−08 | 232 | 462 |
| ‘RETN’ | 0.766 | 0.035172121 | 244 | 414 |
| ‘HLA- | 1.374 | 0.03140587 | 161 | 414 |
| DQA1’ | | | | |
| ‘HLA-DPB1’ | 0.785 | 0.003235049 | 227 | 388 |
| ‘IFI27’ | 4.638 | 3.56E−04 | 15 | 377 |
| ‘HLA- | 1.224 | 0.018823029 | 159 | 365 |
| DRB3’ | | | | |
| ‘C1QC’ | 1.551 | 0.002547556 | 126 | 365 |
| ‘ALOX5AP’ | 0.793 | 0.002016945 | 210 | 364 |
| ‘CTSB’ | 1.920 | 6.81E−09 | 96 | 363 |
| ‘BRI3’ | 0.724 | 0.00802755 | 216 | 358 |
| ‘ANXA2’ | 1.658 | 9.39E−07 | 106 | 333 |
| ‘C1QB’ | 1.137 | 0.011037205 | 147 | 323 |
| ‘CYBB’ | 0.807 | 0.018453508 | 184 | 322 |
| ‘LGALS3BP’ | 1.001 | 5.08E−05 | 155 | 310 |
| ‘HLA-DMB’ | 0.717 | 0.022417743 | 188 | 307 |
| ‘SOD2’ | 1.145 | 0.017321675 | 129 | 301 |
| ‘CTSH’ | 1.227 | 2.00E−04 | 116 | 271 |
| ‘C1orf162’ | 0.529 | 0.038404809 | 178 | 256 |
| ‘CTSS’ | 0.939 | 1.80E−04 | 126 | 242 |
| ‘EVI2B’ | 0.294 | 0.01888367 | 193 | 236 |
| ‘CD81’ | 1.451 | 0.002330558 | 85 | 235 |
| ‘C1QA’ | 1.342 | 0.002254892 | 91 | 230 |
| ‘PRDX1’ | 1.094 | 0.009710824 | 106 | 227 |
| ‘APP’ | 0.696 | 0.015253176 | 133 | 215 |
| ‘GRINA’ | 0.718 | 0.006989716 | 130 | 214 |
| ‘MX1’ | 2.341 | 0.026353098 | 42 | 212 |
| ‘IL2RG’ | 0.785 | 0.001778062 | 122 | 209 |
| ‘NCF1’ | 0.659 | 0.015338721 | 131 | 207 |
| ‘FLNA’ | 0.372 | 0.011692048 | 160.4000 | 206.4233 |
| ‘LGALS3’ | 1.667 | 5.07E−04 | 62.3833 | 199.3967 |
| ‘ADA2’ | 0.759 | 2.90E−08 | 117.7033 | 197.7367 |

Thus, table 1 shows the top 50 genes that are both most highly expressed in HSPC-pDCs generated with ascorbic acid and significantly upregulated compared to HSPC-pDCs generated without ascorbic acid.

A list of the 50 most upregulated genes in HSPC-pDCs produced with AA was also generated. The inclusion criteria in this list were that the genes were statistically significantly upregulated, and that the genes display a FPKM read count (Fragments Per Kilobase Million; FPKM) higher than 10 in the +AA condition. These 50 genes are listed in Table 2.

TABLE 2
The top 50 genes that were most highly upregulated in HSPC-pDCs
when generated with ascorbic acid (+AA) and statistically
significantly upregulated compared to HSPC-pDCs generated without
AA (−AA), and display a FPKM read number (Fragments Per Kilobase
Million; FPKM) higher than 10 are listed. Shown are the official
gene symbols, the log2 values of the fold-change in gene expression
levels upon addition of ascorbic acid to the medium (log2[+AA/−AA]),
the statistical significance (Q-value) of the observation of
differential expression, and the average relative gene expression
levels measured as Fragments Per Kilobase Million (FPKM) in
both conditions (−AA and +AA).

| | Log2 | Qvalue | −AA | +AA |
|---|---|---|---|---|
| Gene | (+AA/−AA) | (+AA/−AA) | FPKM | FPKM |
| ‘CCL13’ | 6.195 | 8.06E−14 | 0.197 | 14.483 |
| ‘DEFA1’ | 5.728 | 6.38E−05 | 19.907 | 1047.070 |
| ‘ASS1’ | 5.358 | 2.41E−08 | 0.477 | 19.263 |
| ‘LCN2’ | 5.282 | 1.39E−54 | 0.930 | 35.670 |
| ‘ORM1’ | 4.863 | 1.09E−20 | 3.003 | 88.200 |
| ‘DEFA4’ | 4.651 | 1.65E−08 | 39.303 | 975.213 |
| ‘CCL2’ | 4.646 | 5.74E−06 | 7.183 | 179.503 |
| ‘IFI27’ | 4.638 | 3.56E−04 | 14.857 | 376.697 |
| ‘MMP14’ | 4.564 | 1.27E−18 | 1.470 | 34.870 |
| ‘MYL9’ | 4.530 | 9.98E−29 | 0.550 | 12.580 |
| ‘DEFA3’ | 4.431 | 0.001704486 | 27.457 | 593.480 |
| ‘GPNMB’ | 4.418 | 4.79E−08 | 4.760 | 101.280 |
| ‘AXL’ | 4.406 | 9.98E−29 | 1.033 | 21.903 |
| ‘CD14’ | 4.038 | 2.98E−25 | 2.797 | 46.303 |
| ‘LILRB4’ | 3.922 | 3.21E−22 | 1.363 | 19.833 |
| ‘HSD11B1’ | 3.736 | 0.044667374 | 1.240 | 16.590 |
| ‘C3’ | 3.579 | 0.001547846 | 1.227 | 14.750 |
| ‘ADAMDEC1’ | 3.569 | 0.0010135 | 1.127 | 13.453 |
| ‘P2RY11’ | 3.498 | 3.25E−10 | 0.970 | 10.823 |
| ‘MAFB’ | 3.471 | 0.00100367 | 1.023 | 11.443 |
| ‘LAG3’ | 3.418 | 8.08E−20 | 2.770 | 29.953 |
| ‘USP18’ | 3.335 | 0.029120385 | 2.210 | 22.600 |
| ‘NUPR1’ | 3.257 | 8.23E−04 | 1.457 | 14.303 |
| ‘GZMB’ | 3.081 | 3.57E−07 | 3.773 | 31.393 |
| ‘CFB’ | 3.059 | 2.09E−06 | 1.257 | 10.533 |
| ‘CES1’ | 3.002 | 0.03002647 | 6.620 | 52.940 |
| ‘SIGLEC1’ | 2.998 | 0.003740839 | 3.223 | 26.273 |
| ‘XCR1’ | 2.881 | 2.80E−29 | 1.363 | 10.463 |
| ‘CD209’ | 2.774 | 9.84E−27 | 2.460 | 16.407 |
| ‘CTSL’ | 2.759 | 0.031636618 | 3.140 | 21.410 |
| ‘PLA2G7’ | 2.759 | 0.030445407 | 12.103 | 86.573 |
| ‘OLR1’ | 2.719 | 0.030792442 | 1.523 | 10.067 |
| ‘IL4I1’ | 2.589 | 5.63E−07 | 4.657 | 28.410 |
| ‘ST14’ | 2.575 | 1.06E−29 | 1.920 | 11.390 |
| ‘SPON2’ | 2.497 | 6.39E−10 | 4.037 | 22.397 |
| ‘SLC7A7’ | 2.460 | 6.15E−21 | 9.010 | 49.703 |
| ‘DNASE1L3’ | 2.459 | 5.63E−10 | 1.893 | 10.470 |
| ‘SLC15A3’ | 2.417 | 4.84E−06 | 9.753 | 52.340 |
| ‘GIMAP4’ | 2.409 | 3.03E−09 | 3.960 | 20.933 |
| ‘ATF5’ | 2.398 | 2.01E−54 | 24.183 | 129.867 |
| ‘IL32’ | 2.353 | 2.92E−23 | 5.960 | 30.560 |
| ‘CXCL10’ | 2.352 | 1.78E−06 | 36.100 | 183.493 |
| ‘MX1’ | 2.341 | 0.026353098 | 41.507 | 211.673 |
| ‘TLR7’ | 2.326 | 1.13E−20 | 2.483 | 12.300 |
| ‘MPEG1’ | 2.325 | 3.81E−23 | 22.783 | 112.763 |
| ‘ACP5’ | 2.221 | 4.83E−10 | 31.073 | 146.980 |
| ‘FPR3’ | 2.200 | 3.93E−08 | 10.853 | 50.280 |
| ‘CHI3L1’ | 2.178 | 0.024834793 | 108.107 | 495.927 |
| ‘KYNU’ | 2.148 | 2.56E−05 | 6.050 | 26.750 |
| ‘IFI44L’ | 2.109 | 1.82E−05 | 17.913 | 81.280 |

Conclusion

In conclusion, we show that the HSPC-pDCs generated by the method according to the invention express a unique and novel genetic profile compared to HSPC-pDCs generated without ascorbic acid (AA) in the medium.

### Example 9—RNA-Sequencing of HSPC-pDCs

Aim of Study

To determine differences in expression profiles of TLR7 and TLR9 pathway-related genes between HSPC-pDCs generated in the presence or absence of Ascorbic acid (AA).

Material and Methods

HSPCs were thawed and 1×10⁵ cells were seeded in DC medium with or without supplementation of ascorbic acid (AA). Following 16 days of differentiation with or without AA in the medium, pDCs were isolated by immunomagnetic depletion of non-pDCs, and HSPCs-pDCs were then primed for 72 hours with IFN-β/γ. Following priming, HSPC-pDCs were stored in RNAprotect Cell Reagent (Qiagen) at −80 degrees until total RNA was extracted using the RNeasy Plus Micro Kit (Qiagen), which efficiently eliminates genomic DNA without the need for DNase treatment. The total RNA was send to BGI Europe for RNA-seq. Here, a non-stranded & polyA-selected mRNA library was prepared from the total RNA and subjected to PE100 sequencing using the BGISEQ platform. The samples generated on average about 4.84 Gb bases per sample. Low quality reads were filtered and the remaining reads were mapped to the genome with an average mapping ratio with the reference genome at 92.74%, the average gene mapping ratio at 79.90%. In total, 18,412 genes were identified. Gene expression was calculated based on the reads, differentially expressed genes and gene ontology analyses were analyzed on BGI's software analysis platform Dr. Tom.

See also example 1.

Results

We selected 54 genes associated with the TLR7 and TLR9 pathways and analyzed the gene expression levels in HSPC-pDCs generated with or without AA in the medium (table 3).

TABLE 3
Shows the official gene symbols, the log2 values of the fold-
change in gene expression levels upon addition of ascorbic acid
to the medium (log2[+AA/−AA]), and the statistical
significance (Q-value) of the observation of differential expression.

| Gene | log2 (+AA/−AA) | Qvalue (+AA/−AA) |
|---|---|---|
| ‘AP3S2’ | −0.480 | 0.04 |
| ‘CLEC4C’ | 3.491 | 9.44E−06 |
| ‘FCER1G’ | 0.509 | 0.03 |
| ‘IRF7’ | 1.065 | 3.19E−04 |
| ‘IRF8’ | 1.594 | 1.77E−23 |
| ‘LAMP5’ | 2.419 | 1.29E−07 |
| ‘LILRA4’ | 1.497 | 0.04 |
| ‘MYD88’ | 0.697 | 0.02 |
| ‘NRP1’ | 1.661 | 0.01 |
| ‘PACSIN1’ | 3.559 | 3.16E−03 |
| ‘PLSCR1’ | 0.576 | 0.03 |
| ‘TLR7’ | 2.326 | 1.1E−20 |
| ‘TLR8’ | 1.584 | 5.99E−07 |
| ‘TLR9’ | 2.487 | 1.70E−07 |
| ‘TRAF3’ | 0.547 | 0.01 |
| ‘UBE2N’ | −0.327 | 0.03 |
| ‘UNC93B1’ | 0.649 | 0.04 |
| ‘AP2A1’ | 0.049 | 0.96 |
| ‘AP3B1’ | −0.122 | 0.73 |
| ‘AP3B2’ | 0.363 | 0.96 |
| ‘AP3D1’ | 0.256 | 0.20 |
| ‘AP3M1’ | −0.050 | 0.96 |
| ‘AP3M2’ | 0.220 | 0.89 |
| ‘AP3S1’ | −0.090 | 0.86 |
| ‘AP4B1’ | −0.275 | 0.53 |
| ‘AP4E1’ | 0.089 | 0.95 |
| ‘AP4M1’ | −0.003 | 1.00 |
| ‘AP4S1’ | −0.313 | 0.49 |
| ‘BLOC1S2’ | 0.114 | 0.94 |
| ‘BST2’ | 0.079 | 0.91 |
| ‘CHUK’ | 0.094 | 0.95 |
| ‘DOCK2’ | 0.098 | 0.71 |
| ‘FURIN’ | 0.089 | 0.93 |
| ‘HSP90B1’ | −0.314 | 0.38 |
| ‘IL3RA’ | 0.247 | 0.75 |
| ‘IRAK1’ | 0.214 | 0.19 |
| ‘IRAK4’ | −0.069 | 0.94 |
| ‘IRF1’ | −0.045 | 0.96 |
| ‘IRF3’ | −0.082 | 0.91 |
| ‘IRF5’ | 0.232 | 0.32 |
| ‘IRF9’ | 0.404 | 0.37 |
| ‘MAP3K7’ | −0.194 | 0.46 |
| ‘MAPK1’ | −0.067 | 0.90 |
| ‘RSAD2’ | 1.967 | 0.23 |
| ‘SLC15A1’ | 1.770 | 0.95 |
| ‘SYK’ | −0.104 | 0.82 |
| ‘TAB1’ | −0.152 | 0.71 |
| ‘TAB2’ | 0.208 | 0.43 |
| ‘TBK1’ | 0.039 | 0.96 |
| ‘TEPSIN’ | −0.118 | 0.92 |
| ‘TLR3’ | 1.507 | 0.28 |
| ‘TRAF6’ | 0.008 | 1.00 |
| ‘TYROBP’ | 0.475 | 0.17 |
| ‘UBE2V1’ | −0.100 | 0.77 |

We found that 17 genes were statistically significantly expressed (all were upregulated upon inclusion of AA in the media during HSPC-pDC generation). These genes include AP3S2, CLEC4C, FCER1G, IRF7, IRF8, LAMP5, LILRA4, MYD88, NRP1, PACSIN1, PLSCR1, TLR7, TLR8, TLR9, TRAF3, UBE2N, and UNC93B1.

Conclusion

In conclusion, we show that several TLR7 and TLR9 pathway-related genes are significantly upregulated when HSPC-pDCs are generated with AA in the medium.

This means that AA directly impacts TLR7 and TLR9 pathway genes and offers a mechanistic explanation for why AA is needed in the media to generate functional HSPC-pDCs that are responsive to TLR7 and TLR9 agonists.

### Example 10—SR1/IL-3 Effect

Aim of Study

To determine the effect of SR1 and IL-3 on HSPC-pDC cell growth, phenotype, and functionality.

Materials and Methods

Generation of HSPC-pDCs from CD34+HSPCs

Cord-blood derived CD34+HSPCs were cultured in GMP DC media (CellGenix®) supplemented with the cytokines and growth factors Flt3-L (100 ng/mL), SCG (100 ng/mL), TPO (50 ng/mL), IL-3 (20 ng/mL) (Peprotech). In addition, 1 μM of the small molecule inhibitor StemRegenin 1 (SR1, STEMCELL Technologies), 20 μg/mL streptomycin and 20 U/mL penicillin (Gibco, Life Technologies) and 50 μg/mL of ascorbic acid (Sigma Aldrich) was added. Cells were cultured at 37° C., 95% humidity, and 5% CO₂ for 17 days, medium was replenished every 2-4 day. The last 3 days, ie. from day 14-17, the culture was split in 4 equal parts. One kept in the same media as previously described, one kept in media without SR1, one kept in media without IL-3 and the last kept in media without both SR1 and IL-3. Viability and total cell numbers during expansion and differentiation of HSPCs was determined using a TC20 Automated Cell Counter (Bio-Rad).

Priming of HSPC-pDCs

Priming of HSPC-pDCs was performed as previously described [Laustsen, A., et al 2018]. Regardless of the media used for the last 3 days of differentiation, after 17 days in culture HSPC-pDCs were primed in GMP DC media (CellGenix@) supplemented with 20 μg/mL streptomycin/20 U/mL penicillin (Gibco, Life Technologies), 20 ng/mL IL-3 (Peprotech) and 50 μg/mL of ascorbic acid (Sigma Aldrich). HSPC-pDCs were primed with 250 U/mL IFN-β (PBL Assay Science) and 250 U/mL IFN-γ (Peprotech) or left unprimed. Cells were primed for 24 hours before being phenotypically and functionally characterized.

TLR7 or TLR9 Agonist Activation

To analyze the capacity of HSPC-pDCs to produce type I IFN, 4×10⁴ pDCs were seeded out in 96-well plates in the same media used for priming of the cells. Cells were subsequently stimulated with agonists directed against TLR7 (R837, tlrl-imq, InvivoGen) or TLR9 (CpG-A 2216, tlrl-2216-1, InvivoGen) at a final concentration of 2.5 μg/mL, or TLR7/8 (R484, tlrl-r848, InvivoGen) at a final concentration of 0.5 μg/mL. Twenty hours post stimulation supernatants were harvested and cryopreserved at −20° C. until analysis.

Assessment of IFNα Release

To quantify the cells functionality, we measure the concentration of IFNα in the cell culture supernatant using human IFNα ELISA (MabTech). The assay was performed according to the protocol provided by MabTech. A standard curve ranging from 1000 μg/ml to 3.9 μg/mL was generated and the IFNα concentration in the supernatants quantified by measuring an optical density (OD) at 450 nm and subtracting the OD at 570 nm using the SpectraMax iD5 platereader (Molecular Devices).

Phenotypic Analysis of Cells Using Flow Cytometry

Flow cytometry was used to immunophenotype pDCs. Briefly, cells were spun down and resuspended in 25 μL TruStain FcBlock (Biolegend) diluted in cold FACS buffer (PBS with 2% FCS and 1 mM EDTA) and incubated cold for 15 minutes. Cells were subsequently stained 25 μL with the following antibodies: APC anti-human Lineage Cocktail (CD3 (UCHT1), CD14 (HCD14), CD16 (3G8), CD19 (HIB19), CD20 (2H7) and CD56 (HCD56), 348801, BioLegend), FITC anti-human CD11c (3.9, 301604, BioLegend), BV421 CD14 (HCD14, 325628, BioLegend), PE CD34 (581, 343506, BioLegend), APC-eFluor780 anti-human CD123 (6H6, 47-1239-42, eBioscience), PE-Cy7 anti-human CD303 (201a, 354214, BioLegend). Cells were stained for 30 min before being washed two times in FACS buffer and finally resuspended in 200 μL FACS Buffer. Fluorescence intensities were measured using the Attune NxT Flow Cytometer equipped with 4 lasers (405 nm, 488 nm, 561 nm, and 638 nm). Data analysis was done using FlowJo (Version 10.8.1, Tree Star, Ashland, OR, USA.

Results

Previously a robust protocol for ex vivo setup for generating high numbers of pDCs from HSPCs under current good manufacturing process (cGMP) has been published. Using a combination of growth factors, cytokines, and small molecules (Flt3-L, TPO, SCF, IL-3, SR1, and L-AA), a solid HSPC expansion and differentiation into immature HSPC-pDCs was shown, and even further it was showed that to generate a mature and functional phenotype, the HSPC-pDCs culture required priming by exposure to type I and II IFNs [Laustsen et al. 2018; Laustsen et al. 2021].

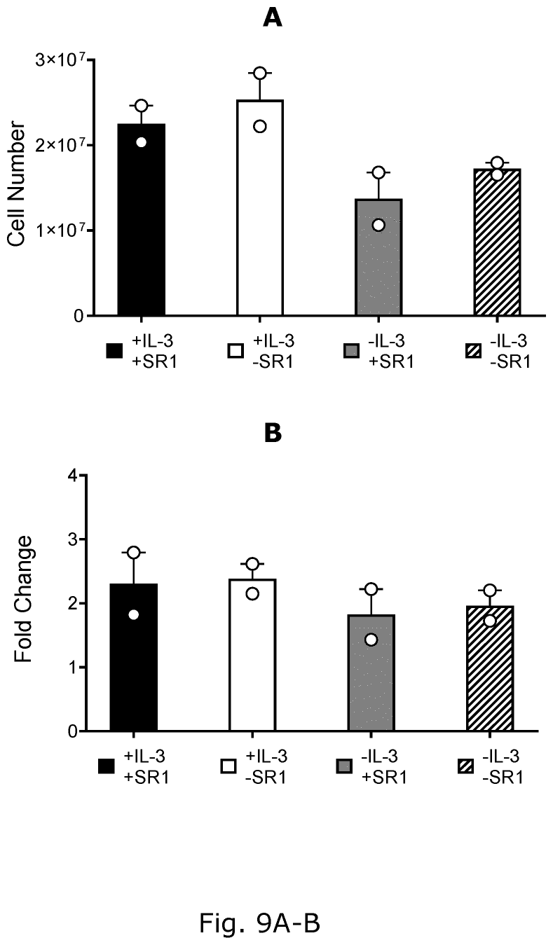

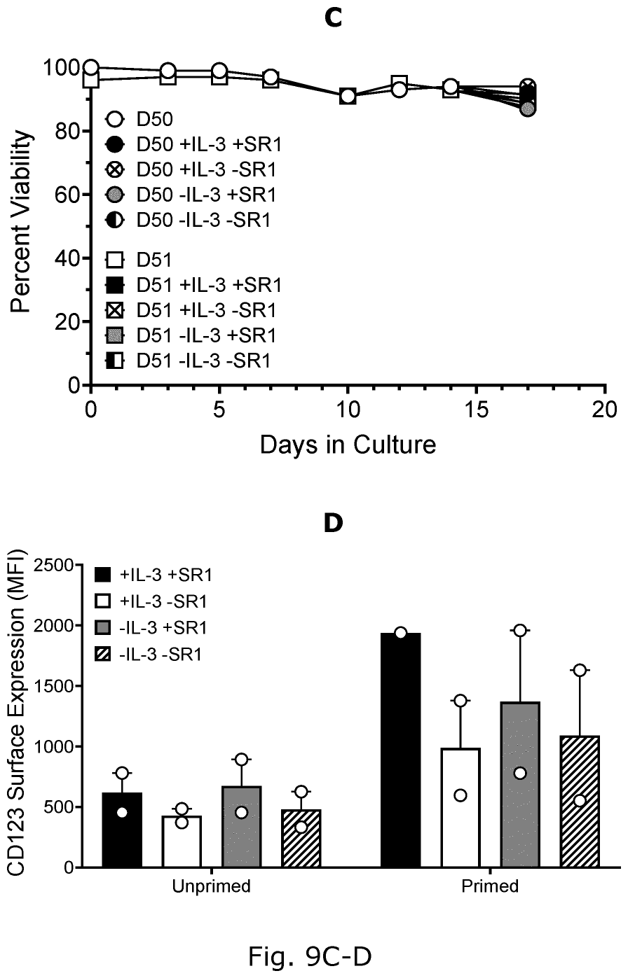

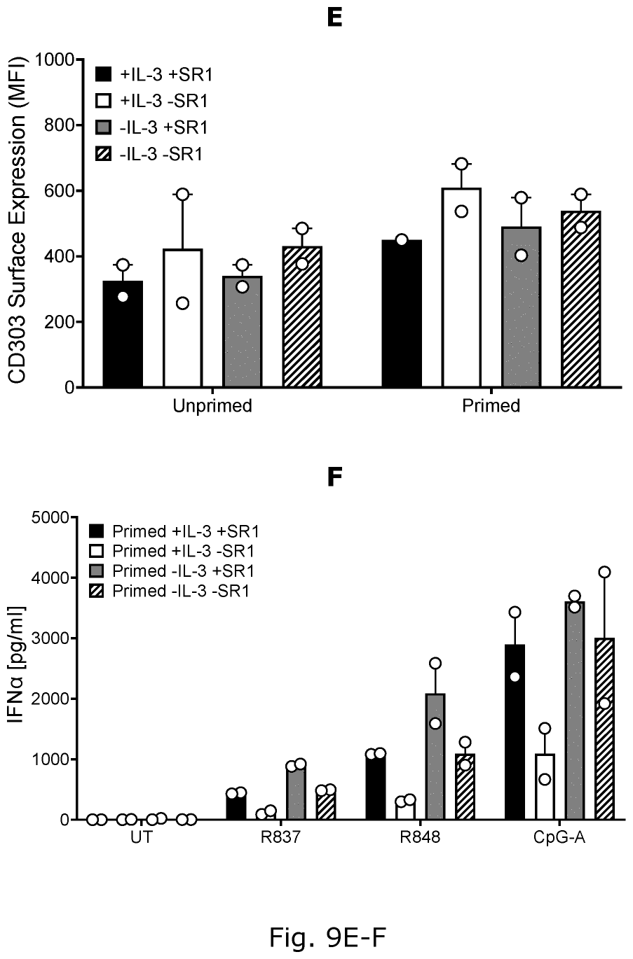

Here we set out to elucidate the effect of SR1 and IL-3 in the final stage of HSPC-to-HSPC-pDC differentiation on both cell growth and viability (FIG. 9A-C), as well as phenotype (FIG. 9D-E) and functionality (FIG. 9F) of the cells.

Neither IL-3 nor SR1 have a great impact on viability ranging from 87% to 94% viable cells on the final day of culture after 3 days in supplement derived media. (FIG. 9C).

When IL-3 is removed from the media for 3 days, we see a decrease in the cell growth, as the difference in the number of cells in culture between day 14 and 17 are 13.7×10⁶ (±3.08×10⁶) cells compared to 22.5×10⁶ (±2.15×10⁶) cells for the standard condition with both IL-3 and SR1 supplemented in the media. Oppositely we see withdrawal of SR1 from the media results in a slight increase in cell growth, being 25.3×10⁶ (±3.12×10⁶) cells (FIG. 9A).

This corresponds fold change in number of cells in culture after the 3 days of culturing under supplement deprivation of 1.85 (±0.40) when removing IL-3, 2.38 (±0.23) when removing SR1 and 2.31 (±0.49) under standard condition (FIG. 9B).

pDCs are known to secrete very large amounts of type I IFNs in response to agonists directed against TLR7 or TLR9 [Swiecki et al., 2015]. Previously, we have shown that HSPC-pDCs require a priming by adding type I and II IFNs to the culture medium to become functionally active and responsive to TLR7 and TLR9 agonists [Laustsen et al., 2018]. We therefore first primed HSPC-pDCs for 24 hours, and subsequently stimulated the cells with TLR7 and TLR9 agonists for 20 hours. Phenotypic analysis of pDC surface markers by flow cytometry of the primed and unprimed HSPC-pDCs (Lin⁻CD11c⁻) show an increase in CD123 expression following priming across all 4 different culturing conditions. Removal of SR1 from the media leads to a decreased CD123 surface expression MEI 988 (±391), compared to the standard culturing condition MEI 1938. (FIG. 9D). However, no apparent difference was observed in CD303 surface expression (FIG. 9E). After TLR stimulation the HSPC-pDCs responsiveness to 3 different TLR agonist are measured with IFNα ELISA on the supernatant from the stimulated cells. Across all 3 TLR agonist we observe a tendency towards removal of SR1 impairs the responsiveness and thus the INFα levels in the supernatants. Most apparent in cells stimulated with the TLR9 agonist CpG-A, where cells kept under the standard culturing condition secretes 2895.91 (±534.17) μg/mL IFNα whereas cells deprived of SR1 secretes 1088.98 (±422.58) μg/mL.

Conclusion

The present example shows that SR1 have a great impact on HSPC-pDC function and phenotype, removal leads to a great decrease in HSPC-pDC function. IL-3 have a greater influence on cell growth, removal impairs cell growth.

### Example 11—HSPC-pDCs Maintain Phenotype and Functionality after Cryopreservation

Aim of Study

To investigate the effect of cryopreservation and thawing of HSPC-pDCs on immunophenotype and functionality.

Materials and Methods

Generation of HSPC-pDCs from CD34+HSPCs

Cord-blood derived CD34+HSPCs were cultured in GMP DC media (CellGenix@) supplemented with the cytokines and growth factors Flt3-L (100 ng/mL), SCF (100 ng/mL), TPO (50 ng/mL), IL-3 (20 ng/mL) (Peprotech). In addition, 1 μM of the small molecule inhibitor StemRegenin 1 (SR1, STEMCELL Technologies), 20 μg/mL streptomycin and 20 U/mL penicillin (Gibco, Life Technologies) and 50 μg/mL of ascorbic acid (Sigma Aldrich) was added. Cells were cultured at 37° C., 95% humidity, and 5% CO₂ for 16-17 days, medium was replenished every 2-3 days. Total cell numbers during expansion and differentiation of HSPCs was determined using a TC20 Automated Cell Counter (Bio-Rad). On day 16-17 a subset of the cells was cryopreserved in CryoStor CS10 medium (StemCell Technologies). HSPC-pDCs were cryostored at density of 5e6 to 50e6 cells/mL in cold (2-8° C.) CryoStor CS10. Following addition of CS10, cells were pre-incubated at 2-8° C. for 5 min, or directly cryopreserved using a slow rate-controlled cooling protocol (approximately −1° C./minute), using either an isopropanol freezing container or Mr. Frosty. Cells were then transferred to −150° C. for prolonged storage. To thaw the HSPC-pDCs, cells were quickly thawed using a 37° C. water-bath. Cells were subsequently washed in 37° C. pre-warmed DC medium and centrifugated at 300×g for 5 min, and resuspended in DC medium supplemented with IL-3 (20 ng/mL), 20 μg/mL streptomycin, 20 U/mL penicillin, and ascorbic acid (50 μg/mL). Following a rest of 2-24 hours, HSPC-pDCs were then washed and directly primed (see next section).

Priming of HSPC-pDCs

HSPC-pDCs were cultured in DC GMP DC media (CellGenix@) supplemented with 20 μg/mL streptomycin/20 U/mL penicillin (Gibco, Life Technologies), 20 ng/mL IL-3 (Peprotech) and 50 μg/mL of ascorbic acid (Sigma Aldrich). The cells were primed with by supplementing 500 U/mL IFN-β (PBL Assay Science) and 500 U/mL IFN-γ (Peprotech) to the medium or left unprimed. The cells were primed for 24 hours before being phenotypically and functionally characterized. For the cryopreserved conditions, the cells were thawed and cultured for 2 to 24h prior to priming.

TLR Agonist Activation

To analyze the capacity of HSPC-pDCs to produce type I IFN, 4×10⁴ HSPC-pDCs were seeded out in 96-well plates in the same culture media used for priming of the cells. Cells were subsequently stimulated with agonists directed against TLR7 (R837, tlrl-imq, InvivoGen) or TLR9 (CpG-A 2216, tlrl-2216-1, InvivoGen) at a final concentration of 2.5 μg/mL, or TLR7/8 (R848, tlrl-r848, InvivoGen) at a final concentration of 0.5 μg/mL. Twenty hours post stimulation supernatants were harvested and stored at −20° C. until analysis.

Assessment of IFNα Release

To quantify functional type I IFN, the reporter cell line HEK-blue IFN-α/β was utilized, according to the manufacturer's instructions (InvivoGen). The cell line was maintained in DMEM+Glutamax-1 (Gibco®, Life Technologies), supplemented with 10% heat-inactivated FCS, 100 μg/mL streptomycin and 200 U/mL penicillin (Gibco, Life Technologies), 100 μg/mL normocin (InvivoGen), 30 μg/mL blasticidin (InvivoGen) and 100 μg/mL zeocin (InvivoGen). Cells were passaged using 1× trypsin (Gibco, Life Technologies) and were not passaged more than 20 times. The HEK-blue IFN-α/β cell line has been generated by stable transfection of HEK293 cells to express IRF9 and STAT2. Moreover, the cell line has been modified to express a reporter gene encoding secreted alkaline phosphathase (SEAP) under the control of ISG54 promotor. Activity of SEAP was assessed using QUANTI-Blue (InvivoGen). Color change was measured at an optical density (OD) of 620 nm using the SpectraMax iD3 platereader (Molecular Devices). For the generation of standard curve, hIFNa2 (PBL Assay Science) was used, and ranged from 2 to 500 U/mL.

Phenotypic Analysis of Cells Using Flow Cytometry

Flow cytometry was used to immunophenotype pDCs. Briefly, cells were spun down stained with 100 μl 1:2000 Ghost Dye Red 780 (13-0865-T500, Tonbo Biosciences) for 30 min (4° C., dark). The cells were washed with 100 μL cold FACS buffer and spun down (350×g, 3 min, RT). The cells were resuspended in 50 μL of antibody cocktail. The antibody cocktail was prepared as follows: 1:20 FITC anti-human Lineage Cocktail ((CD3 (UCHT1), CD14 (HCD14), CD16 (3G8), CD19 (HIB19), CD20 (2H7) and CD56 (HCD56)), 348801, BioLegend), 1:50 APC anti-human CD11c (3.9, 301614, BioLegend), 1:20 BV650 anti-human CD123 (6H6, 306020, BioLegend), 1:20 PE-Cy7 anti-human CD303 (201a, 354214, BioLegend), 1:20 BV421 anti-human CD304 (12C2, 354514, BioLegend) in FACS buffer (PBS with 2% FCS and 1 mM EDTA). Cells were stained for 30 min before being washed two times in FACS buffer and finally resuspended in 200 μL FACS Buffer. Fluorescence intensities were measured using the CytoFlex flow (Beckman Coulter). Data analysis was done using FlowJo (Version 10, Tree Star, Ashland, OR, USA).

Results

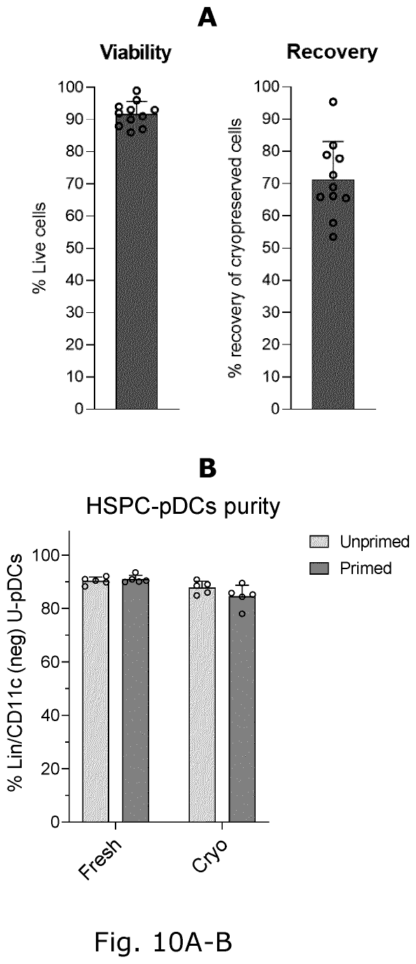

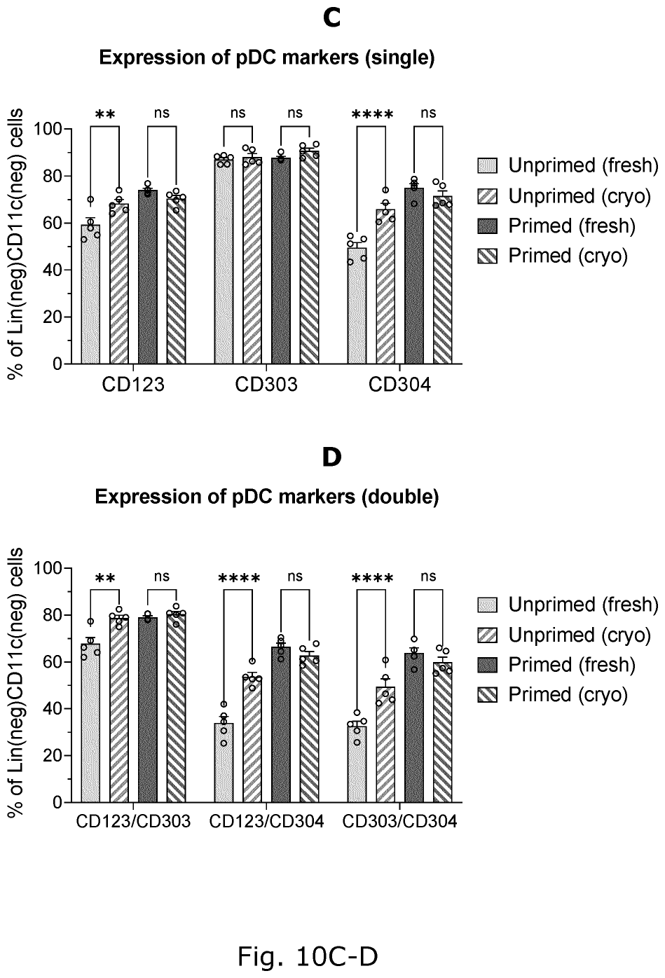

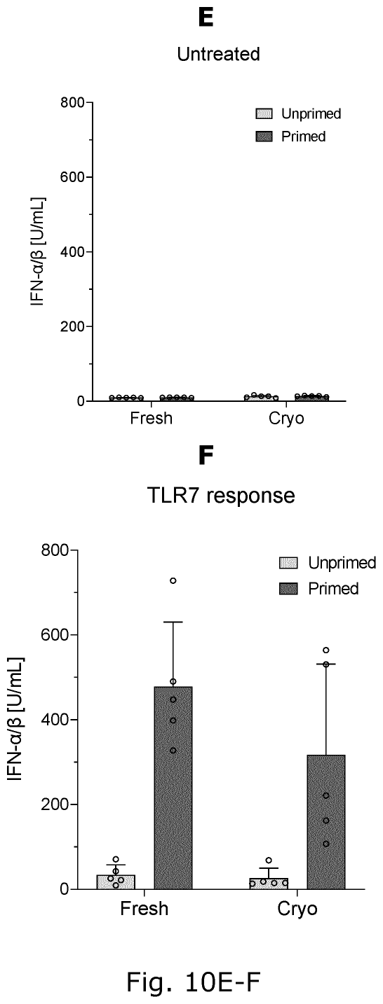

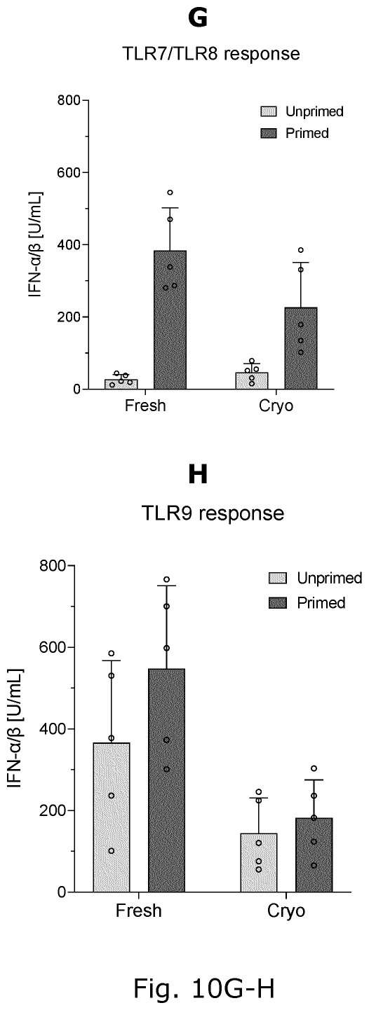

We investigated the feasibility of cryopreserving HSPC-pDCs after differentiation in terms of cell viability, recovery, phenotype, and functionality. We found that cryopreserved HSPC-pDCs could be cryopreserved for long-term storage and thawed with a good average cell recovery of 71% and cell viability of 92% (FIG. 10A). We next compared priming and the immunophenotype of freshly generated HSPC-pDCs and thawed HSPC-pDCs. FIG. 10B shows that thawed HSPC-pDCs (primed/unprimed) display similar purity (lineage⁻, CD11c⁻) to freshly generated primed/unprimed HSPC-pDCs (primed/unprimed). Additionally thawed primed/unprimed HSPC-pDCs express pDC markers (CD123, CD303, and CD304) at a similar or higher frequency compared with freshly generated primed/unprimed HSPC-pDCs (FIG. 10C-D).

We have previously shown that HSPC-pDCs require a priming by adding type I and II IFNs to the culture medium to become functionally active and responsive to TLR7, TLR8, and TLR9 agonists [Laustsen et al., 2018]. We therefore stimulated the cells with TLR7, TLR8, and TLR9 agonists to evaluate their functionality. Importantly, priming does not on its own elicit an IFN-response from neither fresh nor thawed HSPC-pDCs (FIG. 10E). Priming was essential to induce an IFN-response upon TLR7 and/or TLR8 stimulation for both fresh and thawed HSPC-pDCs (FIG. 10F-G). Furthermore, priming plus TLR-stimulation lead to similar IFN-responses from fresh and thawed HSPC-pDCs (FIG. 10F-H).

Conclusion

The present example shows that HSPC-pDCs can be cryopreserved for long-term storage, thawed, and primed without greatly impacting their phenotype and functionality compared with fresh HSPC-pDCs.

### Example 12—HSPC-pDCs can be Primed Prior to Cryopreservation

Aim of Study

The purpose of this study is to test the feasibility of priming of HSPC-pDCs prior to cryopreservation to achieve a ready off-shelf product.

Materials and Methods

Generation of HSPC-pDCs and Priming

Cord-blood derived CD34+HSPCs differentiated into HSPC-pDCs as described in example 11 for 16 days. On day 15, a fraction of the cell culture was supplemented with 250 U/mL IFN-β (PBL Assay Science) and 250 U/mL IFN-γ (Peprotech) for 24h for pre-priming. The other fraction was left untreated. On day 16, both conditions were cryopreserved as described in Example 11. Cryopreserved cells were thawed and cultured in DC GMP DC media (CellGenix@) supplemented with 20 μg/mL streptomycin/20 U/mL penicillin (Gibco, Life Technologies), 20 ng/mL IL-3 (Peprotech) and 50 μg/mL of ascorbic acid (Sigma Aldrich). HSPC-pDCs (standard) were thawed and cultured for 24h and either primed with by supplementing 250U/mL IFN-β (PBL Assay Science) and 250 U/mL IFN-γ (Peprotech) to the medium or left unprimed. The cells were primed for 24 hours before being phenotypically and functionally characterized. Pre-primed cells were thawed and cultured 24h prior to phenotypical and functional analysis.

TLR Agonist Activation

As in Example 11.

Assessment of IFNα Release

As in Example 10.

Phenotypic Analysis of Cells Using Flow Cytometry

Flow cytometry was used to immunophenotype pDCs. Briefly, cells were spun down and resuspended in 1:20 TruStain FcBlock (BioLegend) diluted in cold FACS buffer (PBS with 2% FCS and 1 mM EDTA) and incubated cold for 15 minutes. Cells were subsequently stained with the following antibodies: 1:500 ZombieRed (423110, Biolegend) 1:20 FITC anti-human Lineage Cocktail (CD3 (UCHT1), CD14 (HCD14), CD16 (3G8), CD19 (HIB19), CD20 (2H7) and CD56 (HCD56)) (348801, BioLegend), 1:20 FITC anti-human CD11c (3.9, 301604, BioLegend), 1:20 APC-eFluor780 anti-human CD123 (6H6, 47-1239-42, eBioscience), 1:20 PE-Cy7 anti-human CD304 (12C2, 354508, BioLegend), 1:20 PE anti-human CD40 (5C3, 334308, BioLegend), 1:20 BV421 anti-human CD80 (2D10, 305222, BioLegend), 1:20 BV650 anti-human CD86 (IT2.2, 305428, BioLegend). Cells were stained for 30 min before being washed two times in FACS buffer and finally resuspended in 200 μl fixation buffer (0.9% paraformaldehyde in PBS). Fluorescence intensities were measured using the Attune NxT Flow Cytometer equipped with 4 lasers (405 nm, 488 nm, 561 nm, and 638 nm). Data analysis was done using FlowJo (Version 10.8.1, Tree Star, Ashland, OR, USA.

Results

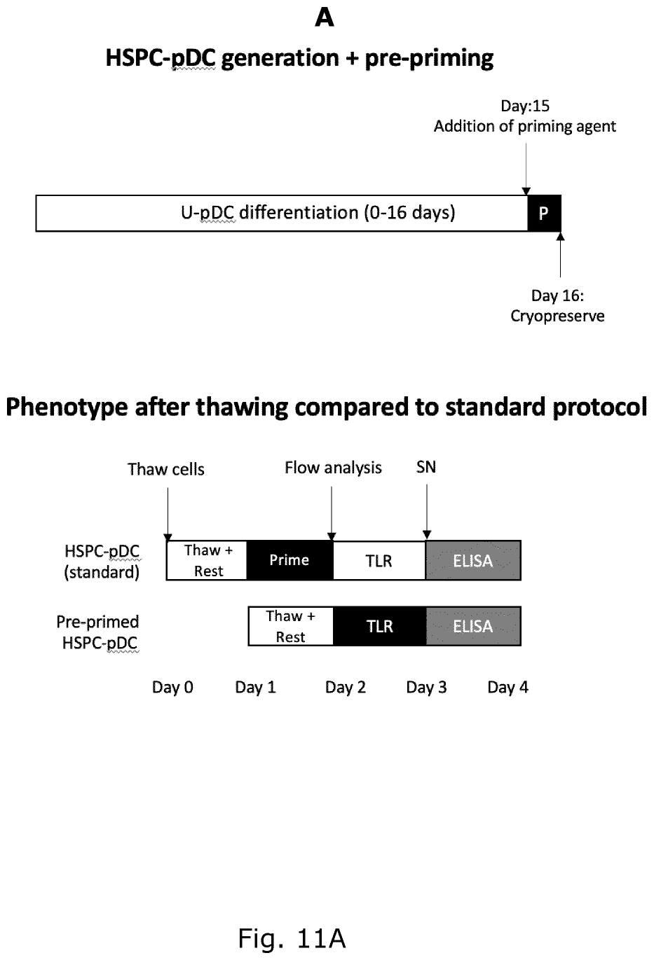

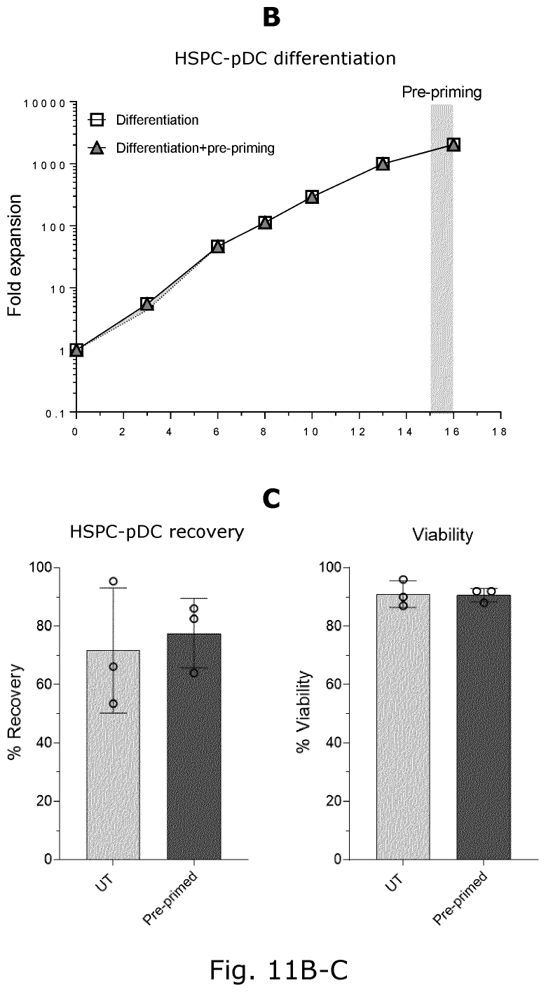

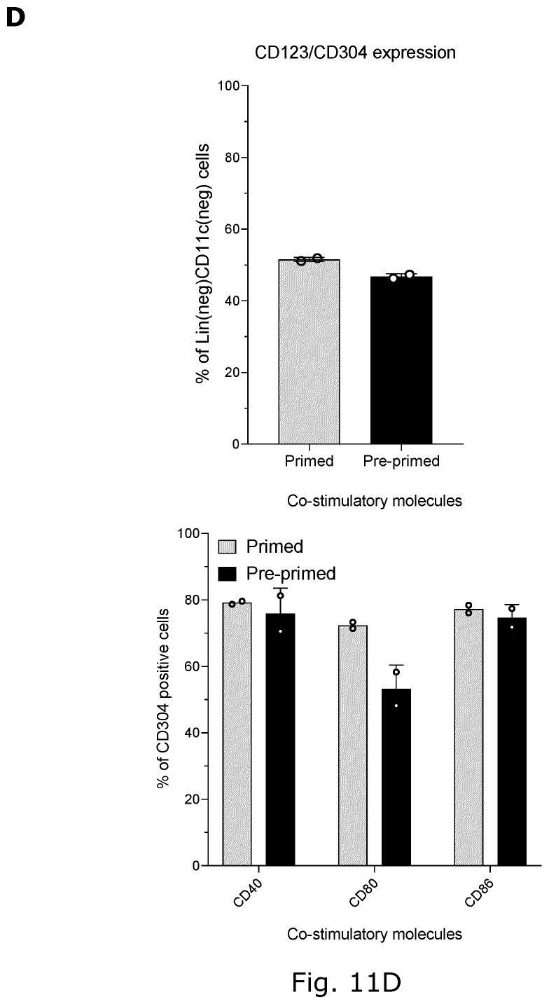

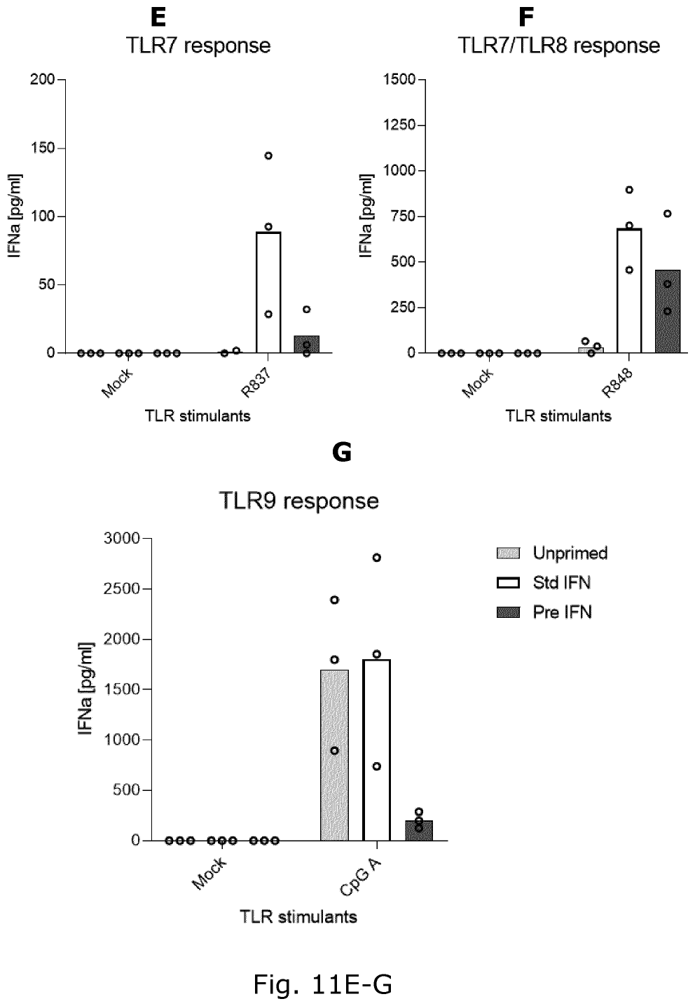

We investigated the feasibility of priming of HSPC-pDC before cryopreservation to untreated cryopreserved HSPC-pDCs in terms of cell viability, phenotype, and functionality. Cord blood HSPCs were differentiated into HSPC-pDC over the course of 16 days. At day 15 a subset of the culture was primed (pre-primed) with IFNs in the differentiation medium. Bulk pre-primed or unprimed HSPC-pDCs were harvested after 16 days of culture and cryopreserved for later phenotypical analysis (FIG. 11A). The pre-priming of the cells of the final day of differentiation culture did not negative impact the cell expansion (FIG. 11B). Upon thawing, we found that HSPC-pDCs primed prior to cryopreservation (pre-primed) had comparable viability and recovery at thawing to untreated cryopreserved HSPC-pDCs (FIG. 11C).

FIG. 11D further shows that pre-primed HSPC-pDC maintain a similar immunophenotype to HSPC-pDC primed after thawing. Thus, pre-primed HSPC-pDCs maintain high expression of pDC-markers (CD123 and CD303) and co-stimulatory molecules (CD40, CD80, and CD80) (FIG. 11D). We have previously shown that HSPC-pDCs require a priming by adding type I and II IFNs to the culture medium to become functionally active and responsive to TLR7, TLR8, and TLR9 agonists [Laustsen et al., 2018]. We therefore stimulated the cells with TLR7, TLR8, and TLR9 agonists to evaluate their functionality. We found that pre-primed HSPC-pDC upon TLR-stimulation had the ability to elicit an IFN-response (FIG. 11E).

Conclusion

This example shows that HSPC-pDCs can be primed prior to cryopreservation and thawed while maintaining their phenotype and the ability to respond to TLR stimulation.

### REFERENCES

Laustsen, A., et al., Interferon priming is essential for human CD34+ cell-derived plasmacytoid dendritic cell maturation and function. Nat Commun, 2018. 9(1): p. 3525.
Laustsen, A., et al., Ascorbic acid supports ex vivo generation of plasmacytoid dendritic cells from circulating hematopoietic stem cells. eLife, 2021. 10:e65528. Swiecki, M. and Colonna, M., The multifaceted biology of plasmacytoid dendritic cells. Nat Rev Immunol, 2015. 15(8): p. 471-85.

Discussion of Data

Here we developed a new, robust, and simplified procedure for generating therapeutically relevant numbers of HSPC-pDCs from very limited numbers of HSPCs using CGMP-compliant medium. We found that differentiating HSPC-pDCs at reduced density and the supplementation of ascorbic acid (AA) to the CGMP medium was key for achieving high and consistent numbers of highly functional HSPC-pDCs. Importantly, we showed that HSPC-pDCs could be generated ex vivo using HSPCs from whole blood. Collectively, our findings lay the foundation for further clinical exploration of pDCs for use as a cellular immunotherapy.

In the last decade, immunotherapy has emerged as a powerful strategy to treat multiple diseases. As the field has attracted a considerable interest from big pharma, the demand for developing new methods and strategies is increasing. pDCs have received much attention owing to their multifaceted role in the immune system, and therapies that selectively activate pDCs, e.g. TLR agonist, have proven to be effective in anti-tumoral therapy. However, while the importance of studying and modulating pDCs for therapy has become more evident, the progress has also been hampered by the low number of cells that can be extracted from the blood. This has also limited the use of pDCs for immunotherapy, but importantly, two independent clinical trials using adoptive transfer of autologous pDCs has shown clinical benefit [5, 6]. In one phase I clinical trial, Tel et al. vaccinated stage IV melanoma patients with autologous pDCs loaded with tumor peptides derived from the melanoma-associated antigens, gp100 and tyrosinase. The therapy improved overall survival, with 45% of patients still being alive after two years, compared to 10% in the matched control patients treated with conventional chemotherapy [5]. Similarly, Westdorp et al. found in a phase Ha clinical trial that vaccination using cDCs and pDCs in combination, loaded with the tumor-associated antigens NY-ESO-1, MAGE-C2 and MUC1, improved the clinical outcome of patients with castration-resistant prostate cancer [6]. In both clinical trials, antigen-loaded pDCs were found to effectively induce B and CD8⁺ T cell anti-tumor specific responses in patients, while being well-tolerated and safe (grade 1-2 toxicities). [5, 6].

The principal drawback, which was also highlighted in these studies, was the maximum feasible dose of only 0.3-3×10⁶ pDCs per vaccination, and only three vaccinations at biweekly intervals were performed [5, 6]. In contrast, in clinical trials utilizing monocyte-derived cDCs (moDCs), patients received four to eight vaccine regiments with biweekly intervals with predefined moDC doses ranging from 10-30×10⁶ cells [14-16]. Similarly, the FDA-approved autologous dendritic cell immunotherapy, Provenge (sipuleucel-T), comprises three vaccination doses of a minimum of 50 million dendritic cells each, and some patients have been treated with doses as high as 1.3 billion dendritic cells [17].

Consequently, there is an unmet need for a clinical manufacturing protocol that allows high and robust numbers of pDCs to be generated. We believe that our platform meets this need by allowing the generation of consistent high numbers of autologous HSPC-pDCs, that can be used for multiple vaccine regiments, and which can be extracted from easily accessible whole blood. We and other groups have previously demonstrated, that pDCs can be generated using CB CD34⁺ HSPCs or mobilized peripheral blood CD34⁺ HSPCs (mPB-HSPCs) [9, 18-22]. While CB HSPCs possess a great stem cell potential, the major drawback is the need for a HLA match between donor and recipient. Obtaining mPB-HSPCs requires multiple injections of granulocyte colony stimulating factor (G-CSF) usually over four consecutive days, followed by apheresis and large-scale CD34 immunomagnetic selection. Thus, the procedure is time-consuming, costly, requires access to expensive equipment, and is associated with inconvenience to the donor and side effects such as bone pain. For research studies of pDC biology, the same challenges apply. Furthermore, cord blood or mobilized peripheral blood is not easily available for common research laboratories. Natural pDCs from peripheral blood have been used, but very few numbers can be isolated from a buffy coat, and the cells have a half-life of only about 24 hours when cultured ex vivo [23]. We have previously shown that generated HSPC-pDCs show superior survival versus peripheral blood-derived pDCs, indicating that HSPC-pDCs are more suitable to receive modifications, e.g. antigen loading and activation, which are required for immunotherapy in a clinical setting. Thordardottir et al. previously reported a yield of 1.6 million HSPC-pDCs starting from 100,000 mPB-HSPCs [9].

Using the same starting cell number and same duration of pDC differentiation, with no pre-expansion included, we generated an average of 306 million HSPC-pDCs amounting to a 191-fold improvement, albeit their starting material was mPB-HSPCs and ours CB-HSPCs.

Importantly, we found that isolating pDCs earlier improved their capacity to produce type I IFN upon stimulation with synthetic TLR7 or TLR9 agonists. This indicates that prolonged pDC differentiation leads to a dysfunctional type I IFN response, possibly due to pDC exhaustion, albeit more research needs to be conducted to further characterize this phenotype. Using our shortened 16-day differentiation protocol and no HSPC pre-expansion, we generated up to 152 million HSPC-pDCs from 100,000 CB-HSPCs.

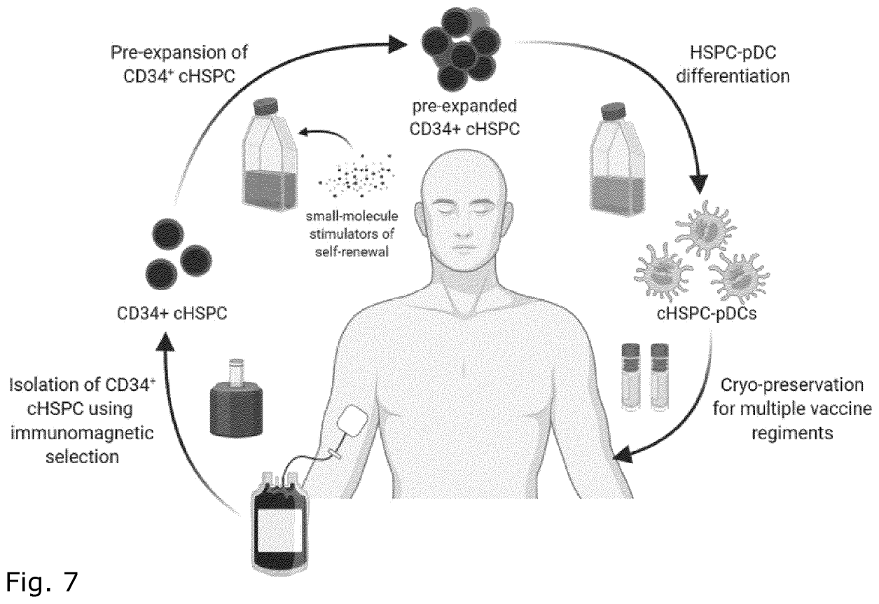

We believe our findings can be used for CGMP-compliant manufacturing of clinically relevant numbers of autologous pDCs from a standard unit of whole blood (FIG. 7). Using our pre-expansion strategy, we were able to generate an average of 8 million HSPC-pDCs starting from 100,000 cHSPCs. With an average number of 1.1×10⁶ cHSPCs in a standard buffy coat from 450 mL of blood, this would allow for an average of 88 million HSPC-pDCs to be generated in a manner that is minimally invasive to the patient. The cHSPC-pDCs can in turn be cryo-preserved, allowing repeated vaccine regiments (FIG. 7). These numbers of generated cells highly exceed the number of natural pDCs that can be obtained from peripheral blood even when using leukapheresis [5, 6]. An additional advantage of HSPC-pDCs over natural pDCs is that HSPC-pDCs are amenable to genetic modifications. This potentially allows CRISPR/Cas gene editing to amplify the response of pDCs or render them resistant to inhibitory tumor signals.

In experiments with CGMP medium, we found that AA highly promoted viability of isolated HSPC-pDCs, and more importantly, that AA supplementation was crucial for the type I IFN response to TLR7 and TLR9 agonists. Our observations indicate a hitherto undescribed key role of AA in pDC innate immune function. Of note, AA is not included in conventional RPMI, but is present within serum, possibly explaining why pDCs generated using RPMI supplemented with fetal-calf serum displayed functional type I IFN responses. AA is highly unstable with sensitivity to temperature, atmospheric oxygen, light, and pH, which may explain some of the observed variations in the type I IFN responses [24].

While AA supplementation also increased HSPC-pDC yield in our setup, the overall percentage of HSPC-pDCs of the total cell population did not increase, indicating that AA does not specifically promote pDC differentiation.

Collectively, we here demonstrate a clinically applicable stem cell differentiation procedure, which we believe can both help elucidate unresolved aspects of pDC biology and facilitate translation of a novel pDC-based treatment modality into clinical immunotherapy.

### REFERENCES

1. Swiecki, M. and M. Colonna, The multifaceted biology of plasmacytoid dendritic cells. Nat Rev Immunol, 2015. 15(8): p. 471-85.
2. van Beek, J. J. P., et al., Human pDCs Are Superior to cDC2s in Attracting Cytolytic Lymphocytes in Melanoma Patients Receiving DC Vaccination. Cell Rep, 2020. 30(4): p. 1027-1038 e4.
3. Wu, J., et al., TLR-activated plasmacytoid dendritic cells inhibit breast cancer cell growth in vitro and in vivo. Oncotarget, 2017. 8(7): p. 11708-11718.
4. Belounis, A., et al., Patients' NK cell stimulation with activated plasmacytoid dendritic cells increases dinutuximab-induced neuroblastoma killing. Cancer Immunol Immunother, 2020. 69(9): p. 1767-1779.
5. Tel, J., et al., Natural human plasmacytoid dendritic cells induce antigen-specific T-cell responses in melanoma patients. Cancer Res, 2013. 73(3): p. 1063-75.
6. Westdorp, H., et al., Blood-derived dendritic cell vaccinations induce immune responses that correlate with clinical outcome in patients with chemo-naive castration-resistant prostate cancer. J Immunother Cancer, 2019. 7(1): p. 302.
7. Ueda, Y., et al., Frequencies of dendritic cells (myeloid DC and plasmacytoid DC) and their ratio reduced in pregnant women: comparison with umbilical cord blood and normal healthy adults. Hum Immunol, 2003. 64(12): p. 1144-51.
8. Zhan, Y., et al., Plasmacytoid dendritic cells are short-lived: reappraising the influence of migration, genetic factors and activation on estimation of lifespan. Sci Rep, 2016. 6: p. 25060.
9. Thordardottir, S., et al., Hematopoietic stem cell-derived myeloid and plasmacytoid DC-based vaccines are highly potent inducers of tumor-reactive T cell and NK cell responses ex vivo. Oncoimmunology, 2017. 6(3): p. e1285991.
10. Laustsen, A., et al., Interferon priming is essential for human CD34+ cell-derived plasmacytoid dendritic cell maturation and function. Nat Commun, 2018. 9(1): p. 3525.
11. Charlesworth, C. T., et al., Priming Human Repopulating Hematopoietic Stem and Progenitor Cells for Cas9/sgRNA Gene Targeting. Mol Ther Nucleic Acids, 2018. 12: p. 89-104.
12. Fares, L., et al., Cord blood expansion. Pyrimidoindole derivatives are agonists of human hematopoietic stem cell self-renewal. Science, 2014. 345(6203): p. 1509-12.
13. Verrax, J. and P. B. Calderon, Pharmacologic concentrations of ascorbate are achieved by parenteral administration and exhibit antitumoral effects. Free Radic Biol Med, 2009.47(1): p. 32-40.
14. Boudewijns, S., et al., Autologous monocyte-derived DC vaccination combined with cisplatin in stage III and IV melanoma patients: a prospective, randomized phase 2 trial. Cancer Immunol Immunother, 2020. 69(3): p. 477-488.
15. Okada, H., et al., Induction of CD8+T-cell responses against novel glioma-associated antigen peptides and clinical activity by vaccinations with {alpha}-type 1 polarized dendritic cells and polyinosinic-polycytidylic acid stabilized by lysine and carboxymethylcellulose in patients with recurrent malignant glioma. J Clin Oncol, 2011. 29(3): p. 330-6.
16. Ribas, A., et al., Multicenter phase II study of matured dendritic cells pulsed with melanoma cell line lysates in patients with advanced melanoma. J Transl Med, 2010. 8: p. 89.
17. Small, E. J., et al., Immunotherapy of hormone-refractory prostate cancer with antigen-loaded dendritic cells. J Clin Oncol, 2000. 18(23): p. 3894-903.
18. Demoulin, S., et al., Production of large numbers of plasmacytoid dendritic cells with functional activities from CD34(+) hematopoietic progenitor cells: use of interleukin-3. Exp Hematol, 2012. 40(4): p. 268-78.
19. Olivier, A., et al., The Notch ligand delta-1 is a hematopoietic development cofactor for plasmacytoid dendritic cells. Blood, 2006. 107(7): p. 2694-701.
20. Curti, A., et al., Stem cell factor and FLT3-ligand are strictly required to sustain the long-term expansion of primitive CD34+DR− dendritic cell precursors. J Immunol, 2001. 166(2): p. 848-54.
21. Thordardottir, S., et al., The aryl hydrocarbon receptor antagonist StemRegenin 1 promotes human plasmacytoid and myeloid dendritic cell development from CD34+ hematopoietic progenitor cells. Stem Cells Dev, 2014. 23(9): p. 955-67.
22. Diaz-Rodriguez, Y., et al., In vitro differentiated plasmacytoid dendritic cells as a tool to induce anti-leukemia activity of natural killer cells. Cancer Immunol Immunother, 2017. 66(10): p. 1307-1320.
23. Grouard, G., et al., The enigmatic plasmacytoid T cells develop into dendritic cells with interleukin (IL)-3 and CD40-ligand. J Exp Med, 1997. 185(6): p. 1101-11.
24. Vojdani, A., et al., New evidence for antioxidant properties of vitamin C. Cancer Detect Prev, 2000. 24(6): p. 508-23.

1. A process for producing HSPC-derived Plasmacytoid dendritic cells (HSPC-pDCs) from hematopoietic stem and progenitor cells (HSPCs), the process comprising the steps:
a) providing hematopoietic stem and progenitor cells (HSPCs);
b) differentiating said HSPCs, to generate precursor-HSPC-pDCs; and
c) priming said precursor-HSPC-pDCs with interferon to provide mature HSPC-pDCs;
wherein, step b) and step c) are carried out in serum-free medium comprising ascorbic acid, preferably the serum-free medium is a CGMP-compliant medium;
wherein step c) further includes the steps of
freezing, storing and thawing the generated precursor-HSPC-pDCs before priming; and/or
freezing the generated precursor-HSPC-pDCs after priming.

2. The process according to claim 1, wherein freezing is conducted by cryopreservation, such as by lowering the temperature to a temperature in the range −80° C. to −196° C.

3. The process according to any of the preceding claims, wherein freezing is conducted before priming.

4. The process according to any of the preceding claims, wherein freezing is conducted after priming.

5. The process according to any of the preceding claims, further comprising the step:
d) activating the mature HSPC-pDCs to induce secretion of one or more cytokines, such as type I and/or III interferon.
wherein step d) is carried out in serum-free medium comprising ascorbic acid, preferably the serum-free medium is a CGMP-compliant medium.

6. The process according to any of the preceding claims, wherein the provided HSPCs in step a) are derived from umbilical cord blood (UCB) or circulating hematopoietic stem and progenitor cells (cHSPCs), preferably wherein in step a), the HSPCs are provided from circulating HSPCs (cHSPC) e.g. found in peripheral blood.

7. The process according to any of the preceding claim, wherein step b) comprises the step b1) and step b2) comprising:
b1) pre-expanding the hematopoietic stem and progenitor cells (HSPCs) provided in step a) starting with a concentration of 0.1-0.5×10⁶ cells/mL for up to 8 days; and
b2) differentiating the pre-expanded cells from step b1) to generate precursor-pDCs.

8. The process according to claim 7, wherein in expansion step b1), cell density is kept in the range 0.1-50×10⁵ cells/mL, such as in the range 0.5-20×10⁵ cells/mL, preferably in the range 1-5×10⁵ cells/mL, such as in the range 5-50×10⁵;
and/or
wherein in expansion step b2), cell density is kept in the range 0.1-50×10⁵ cells/mL, such as in the range 0.5-20×10⁵ cells/mL, preferably in the range 1-5×10⁵ cells/mL, such as in the range 5-50×10⁵.

9. The process according to claim 7 or 8, wherein step b1) is continued for up to 8 days, such as up to 6 days, such as up to 4 days, preferably 4 days;
and/or
wherein step b2) is performed for up to 21 days of culture, such as up to 18 days, preferably up to 16 days of culture.

10. The process according to any of the preceding claims 7-9, wherein the hematopoietic stem and progenitor cells (HSPCs) in step b1) are expanded at least 10 times, such as at least 15 times, such as at least 20 times, or such as at least 25 times.

11. The process according to any of the preceding claims, wherein in priming step c), said priming medium comprises type I and/or type II IFNs, such as comprising subtypes of IFN-α and/or IFN-β and/or IFN-γ, preferably comprising both IFN-β and IFN-γ.

12. The process according to any of claims 5-11, wherein activation step d), is performed
in the presence of an antigen, such as a tumor-associated antigen or a viral antigen in the presence of a TLR7/8 agonist and/or a TLR9 agonist, and/or a STING agonist and/or RIG-I agonist, and/or viral agonist such as Influenza A, Tick-borne encephalitis virus (TBEV), or Herpes simplex virus (HSV); preferably a tumor-associated antigen in the presence of TLR7 agonist and TLR9 agonist;
OR
in the presence of a TLR7/8 agonist and/or a TLR9 agonist, and/or a STING agonist and/or RIG-I agonist, and/or viral agonist such as Influenza A, Tick-borne encephalitis virus (TBEV), or Herpes simplex virus (HSV), preferably TLR7/8 agonist and/or TLR9 agonist.

13. The process according to any of the preceding claims, wherein step b)-c) are performed in the presence of 10-200 μg/mL of ascorbic acid, such as in the range 10-150 μg/mL, such as in the range 10-100 μg/mL, preferably in the range 25-75 μg/mL, more preferably in the range 35-65 μg/mL, or such as around 50 μg/mL of ascorbic acid;
OR
the process according to any of claims 2-9, wherein step b)-d) are performed in the presence of 10-200 μg/mL of ascorbic acid, such as in the range 10-150 μg/mL, such as in the range 10-100 μg/mL, preferably in the range 25-75 μg/mL, more preferably in the range 35-65 μg/mL, or such as around 50 μg/mL of ascorbic acid.

14. HSPC-pDCs obtained/obtainable by a process according to any of the preceding claims; wherein said HSPC-pDCs are cryopreserved.

15. The HSPC-pDCs according to claim 14, wherein said HSPC-pDCs
express one or more genes selected from the group consisting of AP3S2, CLEC4C, FCER1G, IRF7, IRF8, LAMP5, LILRA4, MYD88, NRP1, PACSIN1, PLSCR1, TLR7, TLR8, TLR9, TRAF3, UBE2N, and UNC93B1; and/or
express an increased level of one or more genes selected from the group consisting of AP3S2, CLEC4C, FCER1G, IRF7, IRF8, LAMP5, LILRA4, MYD88, NRP1, PACSIN1, PLSCR1, TLR7, TLR8, TLR9, TRAF3, UBE2N, and UNC93B1 compared to HSPC-pDC isolated from blood; and/or
express an increased level of one or more genes selected from the group consisting of AP3S2, CLEC4C, FCER1G, IRF7, IRF8, LAMP5, LILRA4, MYD88, NRP1, PACSIN1, PLSCR1, TLR7, TLR8, TLR9, TRAF3, UBE2N, and UNC93B1 compared to HSPC-pDCs produced from hematopoietic stem and progenitor cells (HSPCs), in the absence of ascorbic acid; and/or
express an increased level of the one or more genes selected from the group consisting of AP3S2, CLEC4C, FCER1G, IRF7, IRF8, LAMP5, LILRA4, MYD88, NRP1, PACSIN1, PLSCR1, TLR7, TLR8, TLR9, TRAF3, UBE2N, and UNC93B1 compared to HSPC-pDCs produced in the absence of ascorbic acid in step b) or c) or d), such as compared to HSPC-pDCs produced in absence of ascorbic acid in steps b)-d).

16. Isolated HSPC-pDC cells, which
express one or more genes selected from the group consisting of AP3S2, CLEC4C, FCER1G, IRF7, IRF8, LAMP5, LILRA4, MYD88, NRP1, PACSIN1, PLSCR1, TLR7, TLR8, TLR9, TRAF3, UBE2N, and UNC93B1; and/or
express an increased level of one or more genes selected from the group consisting of AP3S2, CLEC4C, FCER1G, IRF7, IRF8, LAMP5, LILRA4, MYD88, NRP1, PACSIN1, PLSCR1, TLR7, TLR8, TLR9, TRAF3, UBE2N, and UNC93B1 compared to HSPC-pDC isolated from blood; and/or
express an increased level of one or more genes selected from the group consisting of AP3S2, CLEC4C, FCER1G, IRF7, IRF8, LAMP5, LILRA4, MYD88, NRP1, PACSIN1, PLSCR1, TLR7, TLR8, TLR9, TRAF3, UBE2N, and UNC93B1 compared to HSPC-pDCs produced from hematopoietic stem and progenitor cells (HSPCs), in the absence of ascorbic acid; and/or
express an increased level of the one or more genes selected from the group consisting of AP3S2, CLEC4C, FCER1G, IRF7, IRF8, LAMP5, LILRA4, MYD88, NRP1, PACSIN1, PLSCR1, TLR7, TLR8, TLR9, TRAF3, UBE2N, and UNC93B1 compared to HSPC-pDCs produced in the absence of ascorbic acid in step b) or c) or d), such as compared to HSPC-pDCs produced in absence of ascorbic acid in steps b)-d);
wherein said HSPC-pDCs are cryopreserved.

17. The HSPC-pDCs according to any of claims 14-16 for use as a medicament.
